(19) United States
(12) Patent Application Publication
(10) Pub. No.: US 2026/0000759 A1
(43) Pub. Date: Jan. 1, 2026

(54) METHODS FOR TREATING PULMONARY FIBROTIC DISEASES OR DISORDERS WITH AN ANTI-ONCOSTATIN M RECEPTOR BETA ANTIBODY

(71) Applicant: Genentech, Inc., South San Francisco, CA (US)

(72) Inventors: Kenta YOSHIDA, South San Francisco, CA (US); Mark Stephen WILSON, South San Francisco, CA (US); Lin PAN, South San Francisco, CA (US); Rojo A. RATSIMANDRESY, South San Francisco, CA (US); Joshua Mark GALANTER, South San Francisco, CA (US)

(21) Appl. No.: 19/320,631

(22) Filed: Sep. 5, 2025

Related U.S. Application Data
(63) Continuation of application No. PCT/US2024/018800, filed on Mar. 7, 2024.
(60) Provisional application No. 63/488,933, filed on Mar. 7, 2023.

(51) Int. Cl. A61K 39/395; A61K 39/00; A61P 7/00; C07K 16/24; C07K 16/28

(52) U.S. Cl. CPC A61K 39/3955; A61P 7/00; C07K 16/248; C07K 16/2866; A61K 2039/507; A61K 2039/545

(57) ABSTRACT

The disclosure provides methods for treating pulmonary fibrotic disorders, including idiopathic pulmonary fibrosis, by administering to patients therapeutically effective doses and dosing regimens of an anti-OSM receptor β antibody, such as vixarelimab, or anti-OSM receptor β antibody, such as vixarelimab, in combination with an anti-IL-6 antibody or an anti-IL-6 receptor agonis, such as tocilizumab.

### CROSS-REFERENCE TO RELATED APPLICATIONS

This application is a continuation of International Patent Application No. PCT/US2024/018800, filed Mar. 7, 2024, which claims the benefit of U.S. Provisional Application No. 63/488,933, filed Mar. 7, 2023, the contents of each of the foregoing applications are hereby incorporated by reference in their entireties.

### TECHNICAL FIELD

The present disclosure pertains to the field of treatment of pulmonary fibrotic disease, such as idiopathic pulmonary fibrosis (IPF), using an anti-OSMRβ antibody, such as vixarelimab, or a combination of an anti-OSMRβ antibody (e.g., vixarelimab) and an anti-IL-6 receptor antibody (e.g., tocilizumab).

### SEQUENCE LISTING

The instant application contains a Sequence Listing which has been submitted electronically in XML format and is hereby incorporated by reference in its entirety. The XML file containing the Sequence Listing XML is named “000218-0086-101-SL.xml,” was created on Sep. 5, 2025, and is 16,402 bytes in size.

### BACKGROUND

Fibrotic interstitial lung diseases (ILDs) are a heterogeneous group of diffuse parenchymal pulmonary disorders characterized by excessive deposition of extracellular matrix components, leading to irreversible loss of lung function (Wijsenbeek and Cottin, 2020, N Engl J Med, 383:958-968). Connective tissue disease-associated ILDs and idiopathic pulmonary fibrosis (IPF) are two of the most common fibrotic ILDs, with an estimated prevalence of 12.1 and 8.2 cases per 100,000, respectively (Duchemann et al., 2017, Eur Respir J, 50:1602419). Among patients with non-IPF fibrotic ILDs, 30%-40% have a progressive fibrosing course (Wijsenbeek et al., 2019, Curr Med Res Opin, 35:2015-2024), leading to chronic disability and premature death. Recent updates to worldwide society guidelines have established the concept of progressive pulmonary fibrosis, defined as non-IPF fibrotic ILDs that meet at least two of three criteria for progression (worsening symptoms, radiological progression, and physiological progression) occurring within the past year without alternative explanation (Raghu et al. 2022).

Pirfenidone and nintedanib are currently the only pharmacologic therapies approved for the treatment of IPF (Raghu et al., 2022, Am J Respir Crit Care Med, 205: e18-e47). The rate of decline in forced vital capacity (FVC) is slower in patients treated with pirfenidone and nintedanib. However, neither treatment halts disease progression nor improves any objective measurements of disease status (Nathan et al., 2016, Thorax, 71:429-435). Therefore, disease progression and respiratory decline is inevitable. Thus, a need for additional novel treatment approaches remains.

Given the need for therapeutics effective in treating and slowing progression of pulmonary fibrotic disorders, provided herein are methods for using anti-OSMRβ antibodies to treat pulmonary fibrotic disorders such as IPF and SSc-ILD.

### SUMMARY OF THE DISCLOSURE

In a first aspect, a method for administering to a subject in need thereof a therapeutically effective dose of an anti-OSMRβ (oncostatin M receptor) antibody is provided.

In a second aspect, a method for treating a pulmonary fibrotic disease is provided. In some embodiments, the method comprises administering to a subject in need thereof a therapeutically effective dose of an anti-OSMRβ (oncostatin M receptor beta) antibody. In some embodiments, the pulmonary fibrotic disease is selected from the group consisting of progressive pulmonary fibrosis (PPF), idiopathic pulmonary fibrosis (IPF), and systemic sclerosis-interstitial lung disease (SSc-ILD). In some embodiments, the pulmonary fibrotic disease is progressive pulmonary fibrosis (PPF). In some embodiments, the pulmonary fibrotic disease is idiopathic pulmonary fibrosis (IPF). In some embodiments, the pulmonary fibrotic disease is systemic sclerosis-interstitial lung disease (SSc-ILD).

In a third aspect, a method of increasing the FVC in a subject suffering from a pulmonary fibrotic disorder is provided. In some embodiments, the method comprises administering a therapeutically effective dose of anti-OSMRβ antibody to a subject in need thereof.

In a fourth aspect, a method of increasing the distance traveled, as measured the 6MWT, by a subject suffering from a pulmonary fibrotic disorder is provided. In some embodiments, the method comprises administering a therapeutically effective dose of anti-OSMRβ antibody to a subject in need thereof.

In a fifth aspect, a method of reducing cough frequency, as measured a digital continuous ambulatory cough detection device, by a subject suffering from a pulmonary fibrotic disorder is provided. In some embodiments, the method comprises administering a therapeutically effective dose of anti-OSMRβ antibody to a subject in need thereof.

In a sixth aspect, a method for treating an inflammatory disease is provided. In some embodiments, the method comprises comprising administering to a subject in need thereof a therapeutically effective dose of an anti-OSMRβ (oncostatin M receptor beta) antibody.

In some embodiments of any of the above aspects, the anti-OSMRβ antibody inhibits activation of the OSMRβ pathway by oncostatin M (OSM) and/or interleukin-31 (IL-31). In some embodiments of any of the above aspects, the anti-OSMRβ antibody inhibits activation of the OSMRβ pathway by oncostatin M (OSM) and interleukin-31 (IL-31).

In some embodiments of any of the above aspects, the anti-OSMRβ antibody comprises a heavy chain variable domain (VH) comprising SEQ ID NO:7 and a light chain variable domain (VL) comprising SEQ ID NO:8. In some embodiments of any of the above aspects, the anti-OSMRβ antibody comprises a heavy chain (HC) comprising SEQ ID NO:5 and a light chain (LC) comprising SEQ ID NO:6. In some embodiments of any of the above aspects, the anti-OSMRβ antibody is vixarelimab.

In some embodiments of any of the above aspects, the therapeutically effective dose is about 360 mg to 720 mg of the anti-OSMRβ antibody. In some embodiments of any of the above aspects, the therapeutically effective dose is 360 mg of the anti-OSMRβ antibody. In some embodiments of any of the above aspects, the anti-OSMRβ antibody is administered once per week, once every 2 weeks, once every 3 weeks, once every 4 weeks, or once every month. In some embodiments of any of the above aspects, the anti-OSMRβ antibody is administered once per week. In some embodiments of any of the above aspects, the anti-OSMRβ antibody is administered once every 2 weeks. In some embodiments of any of the above aspects, the anti-OSMRβ antibody is administered once every 3 weeks. In some embodiments of any of the above aspects, the anti-OSMRβ antibody is administered once every 4 weeks. In some embodiments of any of the above aspects, the anti-OSMRβ antibody is administered once every month.

In some embodiments of any of the above aspects, the method comprises administering to the subject 360 mg, 540 mg, or 720 mg of the anti-OSMRβ antibody once every 1 week, once every 2 weeks, once every 3 weeks, once every 4 weeks, or once every month. In preferred embodiments of any of the above aspects, the method comprises administering to the subject 360 mg of the anti-OSMRβ antibody about once every 2 weeks, wherein the anti-OSMRβ antibody is vixarelimab.

In some embodiments of any of the above aspects, the subject is not administered a loading dose of the anti-OSMRβ antibody.

In some embodiments of any of the above aspects, the anti-OSMRβ antibody is administered subcutaneously. In other embodiments of any of the above aspects, the anti-OSMRβ antibody is administered intravenously.

In some embodiments of any of the above aspects, the method comprises treating the subject wherein the subject prior to treatment with the anti-OSMRβ antibody has a percentage of predicted forced vital capacity (% FVC) of about 35% to 90%, about 35% to 75%, about 35% to 50%, about 45% to 55%, about 30% to 60%, about 50% to 90%, about 50% to 75%, about 40% to 45%, about 40% to 50%, about 45% to 50%, or about 45% to about 50%. In some embodiments of any of the above aspects, the subject has a FVC of about 45% predicted. In some embodiments of any of the above aspects, the % FVC is measured using spirometry.

In some embodiments of any of the above aspects, the subject has a forced expiratory volume in 1 second (FEV1)-to-FVC ratio of about 0.35 to 0.70, about 0.50 to 0.70, about 0.60 to 0.70, about 0.35 to 0.50, about 0.40 to 0.50, about 0.50 to 0.60, about 0.60 to 0.70, about 0.70 to 0.80, prior to treatment with the anti-OSMRβ antibody. In some embodiments of any of the above aspects, the subject has a FEV1-FVC ratio of about 0.70 to 0.80.

In preferred embodiments of any of the above aspects, the subject has forced vital capacity (FVC) of about 45% predicted or greater and a forced expiratory volume in 1 second (FEV1)-to-FVC ratio greater than about 0.70 prior to treatment with the anti-OSMRβ antibody.

In some embodiments of any of the above aspects, the method comprises treating the subject having a pulmonary fibrotic disorder, wherein administering the dose of anti-OSMRβ antibody to the subject results in a change in FVC in the subject, wherein the change is a measure of absolute change in FVC in milliliters (ml) over a treatment period beginning the time at the first administration of the anti-OSMRβ antibody until the time at which a later dose of the anti-OSMRβ antibody is administered. In some embodiments of any of the above aspects, the change in FVC during the treatment period is a decrease in FVC of less than 25 mL, 50 mL, 75 mL, 100 mL, 125 mL, 150 mL, 175 mL, 200 mL, 225 mL or 250 mL or an increase in FVC of at least 25 mL, 50 mL, 75 mL, 100 mL, 125 mL, 150 mL, 175 mL, 200 mL, 225 mL, or 250 mL. In some embodiments of any of the above aspects, the anti-OSMRβ antibody is administered every 2 weeks at a dose of about 360 mg and the change in FVC during the time period is a decrease in FVC of less than 25 mL, 50 mL, 75 mL, 100 mL, 125 mL, 150 mL, 175 mL, 200 mL, 225 mL or 250 mL or an increase in FVC of at least 25 mL, 50 mL, 75 mL, 100 mL, 125 mL, 150 mL, 175 mL, 200 mL, 225 mL or 250 mL. In some embodiments of any of the above aspects, the treatment period is about 6 weeks, about 12 weeks, about 24 weeks, about 36 weeks, about 48 weeks, about 60 weeks, or about 72 weeks. In some embodiments of any of the above aspects, the treatment period is about 52 weeks.

In some embodiments of any of the above aspects, the method comprises treating the subject having a pulmonary fibrotic disorder, wherein administering the dose of anti-OSMRβ antibody to the subject results in a change in diffusion capacity of the lung for carbon monoxide adjusted for hemoglobin (DL_{CO}[Hb]) in the subject. In some embodiments of any of the above aspects, the method is sufficient to produce an increase in the DLCO[Hb] compared to a baseline, wherein the baseline measurement is taken prior to the administering. In some embodiments of any of the above aspects, the method is sufficient to produce an increase in the DLCO percent (DL_{CO}%) predicted compared to the baseline measurement. In some embodiments of any of the above aspects, the increase in DL_{CO} or DL_{CO} % predicted is at least 5%, 10%, 15%, 20%, 25%, 30%, 35%, 40%, 45%, 50%, 55%, 60%, 65%, 70%, 75%, 80% or 90% above the respective baseline measurement over a treatment period. In some embodiments of any of the above aspects, the treatment period is about 6 weeks, about 12 weeks, about 24 weeks, about 36 weeks, about 48 weeks, about 60 weeks, or about 72 weeks. In some embodiments of any of the above aspects, the treatment period is about 52 weeks.

In some embodiments of any of the above aspects, the method comprises treating the subject having a pulmonary fibrotic disorder, wherein administering the dose of anti-OSMRβ antibody to the subject results in a change in distance traveled by the subject in the 6-minute walk test (6MWT), wherein the change is the difference in the distance traveled by the subject in the 6MWT performed at 2 time points in a treatment period wherein the first time point is at the first administration of the anti-OSMRβ antibody and the second time point is the time at which a later dose of the anti-OSMRβ antibody is administered. In some embodiments of any of the above aspects, the anti-OSMRβ antibody is administered every 2 weeks at a dose of about 360 mg. In some embodiments of any of the above aspects, the difference in the distance traveled by the subject in the 6MWT performed at the 2 time points is a decrease of less than about 5%, 10%, 15%, 20%, 25%, or 30% or is an increase of at least about 5%, 10%, 15%, 20%, 25%, or 30%. In some embodiments of any of the above aspects, the difference in the distance traveled by the subject in the 6MWT performed at the 2 time points is a decrease of less than about 5%, 10%, 15%, 20%, 25%, or 30%. In some embodiments of any of the above aspects, the difference in the distance traveled by the subject in the 6MWT performed at the 2 time points is an increase of at least about 5%, 10%, 15%, 20%, 25%, or 30%. In some embodiments of any of the above aspects, the treatment period is about 6 weeks, about 12 weeks, about 24 weeks, about 36 weeks, about 48 weeks, about 60 weeks, or about 72 weeks. In some embodiments of any of the above aspects, the treatment period is about 52 weeks.

In some embodiments of any of the above aspects, the method comprises treating the subject having a pulmonary fibrotic disorder, wherein administering the dose of anti-OSMRβ antibody to the subject results in a decrease in quantitative lung fibrosis on a high-resolution computed tomography (HRCT) scan in the subject. In some embodiments of any of the above aspects, the method is sufficient to produce a decrease in the quantitative lung fibrosis on HRCT scan compared to a baseline, wherein the baseline measurement is taken prior to the administering. In some embodiments of any of the above aspects, the decrease in the quantitative lung fibrosis on HRCT scan is at least 1%, 2%, 3%, 4%, 5%, 8%, 10%, 12%, 15%, 20%, or 30% below the respective baseline measurement over a treatment period. In some embodiments of any of the above aspects, the treatment period is about 6 weeks, about 12 weeks, about 24 weeks, about 36 weeks, about 48 weeks, about 60 weeks, or about 72 weeks. In some embodiments of any of the above aspects, the treatment period is about 52 weeks.

In some embodiments of any of the above aspects, the method comprises treating the subject having a pulmonary fibrotic disorder, wherein administering the dose of anti-OSMRβ antibody to the subject results in a reduction in cough frequency. In some embodiments of any of the above aspects, the reduction cough frequency is measured by a digital continuous ambulatory cough detection device.

In some embodiments of any of the above aspects, the subject has been diagnosed with or determined to suffer from one or more pulmonary fibrotic disorders. In some embodiments of any of the above aspects, the pulmonary fibrotic disorder is idiopathic pulmonary fibrosis (IPF) or progressive pulmonary fibrosis (PPF) (alternatively referred to as pulmonary fibrosis-interstitial lung disease (PF-ILD)).

In some embodiments of any of the above aspects, the PPF is chronic fibrosing ILD (CF-ILD) with a progressive phenotype, interstitial lung disease (ILD), systemic sclerosis-ILD (SSc-ILD), drug-induced ILD, hypersensitivity pneumonitis, interstitial pneumonia with autoimmune features (IPAF), fibrosing interstitial pneumonia, and unclassifiable ILD. In some embodiments of any of the above aspects, the pulmonary fibrotic disorder is chronic fibrosing interstitial lung diseases with a progressive phenotype.

In some embodiments of any of the above aspects, the pulmonary fibrosis is associated with one or more of the following: usual interstitial pneumonia, idiopathic interstitial pneumonia, desquamative interstitial pneumonia, respiratory bronchiolitis-interstitial lung disease, acute interstitial pneumonia, nonspecific interstitial pneumonia, sarcoidosis, cryptogenic organizing pneumonia, eosinophilic pneumonia, infection, exposure to occupational or environmental agents, cigarette smoking, interstitial lung disease induced by drugs or radiation, rheumatic disease-associated interstitial lung disease, lymphoid interstitial pneumonia, pleuropulmonary fibroelastosis, pulmonary Langerhans cell histiocytosis, systemic sclerosis-interstitial lung disease, Hermansky-Pudlak syndrome, and telomeropathy.

In some embodiments of any of the above aspects, the subject has not been diagnosed with or is not experiencing an inflammatory bowel disease.

In some embodiments of any of the above aspects, the subject has not been diagnosed with or is not experiencing a fibrotic skin disease such as prurigo nodularis (PN) or atopic dermatitis (AD).

In some embodiments of any of the above aspects, the anti-OSMRβ antibody is administered in combination with a second therapeutic agent. In some embodiments of any of the above aspects, the second therapeutic agent is a therapeutic agent indicated for a lung fibrotic disease or disorder. In some embodiments of any of the above aspects, the second therapeutic agent is an anti-fibrotic. In preferred embodiments of any of the above aspects, the second therapeutic agent is pirfenidone or nintedanib.

In some embodiments of any of the above aspects, the anti-OSMRβ antibody is administered before, during, or after administration with the second therapeutic agent. In other embodiments of any of the above aspects, the second therapeutic agent is an anti-IL-6 antibody or an anti-IL-6 receptor agonist. In other embodiments of any of the above aspects, the second therapeutic agent is an anti-IL-6 antibody or an anti-IL-6 receptor antibody. In some embodiments of any of the above aspects, the heavy chain of the anti-IL-6 antibody or the anti-IL-6 receptor antibody comprises the amino acid sequence of SEQ ID NO: 13. In some embodiments of any of the above aspects, the light chain of the anti-IL-6 antibody or the anti-IL-6 receptor antibody comprises the amino acid sequence of SEQ ID NO: 14. In some embodiments of any of the above aspects, the anti-IL-6 antibody or the anti-IL-6 receptor antibody is tocilizumab. In some embodiments of any of the above aspects, the anti-IL-6 antibody or the anti-IL-6 receptor antibody comprises the six CDRs of tocilizumab.

In some embodiments of any of the above aspects, the anti-OSMRβ antibody is administered to the subject after the subject has been treated with the second therapeutic agent for at least 1 week, 1 month, 6 months, 1 year, 3 years, or 5 years.

In a seventh aspect, the present disclosure provides a method of treating a pulmonary fibrotic disease in a subject in need thereof, the method comprising administering to the subject a therapeutically effective amount of (a) an anti-OSMRβ antibody and (b) an anti-IL-6 receptor antibody. In some embodiments, the pulmonary fibrotic disease is selected from the group consisting of progressive pulmonary fibrosis (PPF), idiopathic pulmonary fibrosis (IPF), and systemic sclerosis-interstitial lung disease (SSc-ILD). In some embodiments, the pulmonary fibrotic disease is progressive pulmonary fibrosis (PPF). In some embodiments, the pulmonary fibrotic disease is idiopathic pulmonary fibrosis (IPF). In some embodiments, the pulmonary fibrotic disease is systemic sclerosis-interstitial lung disease (SSc-ILD).

In an eighth aspect, the present disclosure provides a method of treating a lung inflammatory and/or fibrotic disease in a subject in need thereof, the method comprising administering to the subject a therapeutically effective amount of (a) an anti-OSMRβ antibody and (b) an anti-IL-6 receptor antibody.

In some embodiments of any of the above aspects, the anti-OSMRβ antibody comprises a heavy chain variable domain (VH) comprising SEQ ID NO:7 and a light chain variable domain (VL) comprising SEQ ID NO:8. In some embodiments of any of the above aspects, the anti-OSMRβ antibody comprises a heavy chain (HC) comprising SEQ ID NO:5 and a light chain (LC) comprising SEQ ID NO:6. In some embodiments of any of the above aspects, the anti-OSMRβ antibody is vixarelimab. The anti-OSRMβ antibody can be administered at any of the doses disclosed herein, any of the dosing frequencies disclosed herein, or any combination of dose and frequency disclosed herein.

In some embodiments of any of the above aspects, the heavy chain of the anti-IL-6receptor antibody comprises the amino acid sequence of SEQ ID NO: 13. In some embodiments of any of the above aspects, the anti-IL-6 receptor antibody comprises the amino acid sequence of SEQ ID NO: 14. In some embodiments of any of the above aspects, the anti-IL-6 receptor antibody is tocilizumab. In some embodiments of any of the above aspects, the anti-IL-6 receptor antibody comprises the six CDRs of tocilizumab.

In a ninth aspect, the present disclosure provides a method of treating a pulmonary fibrotic disease in a subject in need thereof, the method comprising administering to the subject a therapeutically effective amount of vixarelimab and tocilizumab. In some embodiments, the pulmonary fibrotic disease is selected from the group consisting of progressive pulmonary fibrosis (PPF), idiopathic pulmonary fibrosis (IPF), and systemic sclerosis-interstitial lung disease (SSc-ILD). In some embodiments, the pulmonary fibrotic disease is progressive pulmonary fibrosis (PPF). In some embodiments, the pulmonary fibrotic disease is idiopathic pulmonary fibrosis (IPF). In some embodiments, the pulmonary fibrotic disease is systemic sclerosis-interstitial lung disease (SSc-ILD). Vixarelimab can be administered at any of the doses disclosed herein, any of the dosing frequencies disclosed herein, or any combination of dose and frequency disclosed herein.

In a tenth aspect, the present disclosure provides a method of treating a lung inflammatory and/or fibrotic disease in a subject in need thereof, the method comprising administering to the subject a therapeutically effective amount of vixarelimab and tocilizumab. Vixarelimab can be administered at any of the doses disclosed herein, any of the dosing frequencies disclosed herein, or any combination of dose and frequency disclosed herein.

In some embodiments, the anti-OSMRβ antibody (e.g., vixarelimab) and the anti-IL-6 receptor antibody (e.g., tocilizumab) are administered simultaneously. In some embodiments, the anti-OSMRβ antibody (e.g., vixarelimab) and the anti-IL-6 receptor antibody (e.g., tocilizumab) are administered sequentially. In some embodiments, the anti-OSMRβ antibody (e.g., vixarelimab) is administered before the anti-IL-6 receptor antibody (e.g., tocilizumab). In some embodiments, the anti-OSMRβ antibody (e.g., vixarelimab) is administered after the anti-IL-6 receptor antibody (e.g., tocilizumab). In some embodiments, the anti-OSMRβ antibody (e.g., vixarelimab) and the anti-IL-6 receptor antibody (e.g., tocilizumab) are administered in the same composition. In some embodiments, the anti-OSMRβ antibody (e.g., vixarelimab) and the anti-IL-6 receptor antibody (e.g., tocilizumab) are administered In different compositions.

### BRIEF DESCRIPTION OF THE DRAWINGS

FIG. 1 provides the results of scRNA seq analysis of lung tissue isolated from a human subject diagnosed with idiopathic pulmonary fibrosis shows OSM expression in macrophages and OSMRβ expression in epithelial cells, single smooth muscle cell types (SMC), fibroblasts, and endothelial cells.

FIG. 2A provides the results of treating a mouse model of lung fibrosis with a control antibody or an anti-OSM antibody. FIG. 2B provides the results of antibody treatment on neutrophils in bronchoalveolar lavage fluid from mice sacrificed on Day 24 is shown in the right panel.

FIG. 3A provides the total hydroxyproline (Total OHP, ug/half lung) in lung tissue was measured at day 24. FIG. 3B demonstrates ‘New’ hydroxyproline (New OHP, ug/half lung) in lung tissue was measured in deuterated water treated mice at day 24. Graphs show mean±SD with 5-25 mice per group. P value calculated by t-test. * P<0.05.

FIG. 4A-FIG. 4F demonstrates the results of lung tissue was recovered from mice at day 14 post BLM. FIG. 4A provides the body weight results of weekly monitoring expressed as % of initial weight. FIG. 4B demonstrates the survival rate of mice 25 days post BLM. FIG. 4C shows tissue volume (TV) at day 22 post BLM. RNA was extracted and used for RNA sequencing. FIG. 4D and FIG. 4E provides disease-relevant, tissue remodeling genes and expressed relative to PBS treated control mice. Graphs show individual mice with mean. *P<0.05. FIG. 4F provides the top Ingenuity pathway analyses (IPA) for isotype and anti-OSM treated mice following BLM.

FIG. 5 demonstrates PK/PD following a single IV dose of vixarelimab to determine efficacious concentration (Ceff) in non-human primate (NHP) itch model. Vixarelimab (KPL-716) was administered intravenously (IV) on day 1;6 animals were treated per dose group. Scratching events are calculated as post-IL-31 challenge minus pre-IL-31 challenge events. Lower limit of quantification=0.04 μg/mL Concentrations of vixarelimab at the same dosing regimens were simulated and correlated with reduction in rhIL-31-induced scratching, verifying a C_{eff} threshold of 5-8 μg/mL for inhibiting pruritic responses in this model. The X-axis of each graph corresponds to the number of days post-vixarelimab treatment (Pre-Tx=prior to treatment). The left Y-axis of each graph corresponds to the number of scratching events, which is the pharmacodynamic effect. The right Y-axis of each graph corresponds to the serum vixarelimab (KPL-716) concentration in μg/ml.

FIG. 6 demonstrates the results of the human Phase 1b clinical study of vixarelimab treatment of AD involved IV administration of vixarelimab at doses of 0.3 mg/kg, 1.5 mg/kg, 7.5 mg/kg, 10 mg/kg or 20 mg/kg and SC administration at doses of 1.5 mg/kg or 360 mg. Patients were monitored for safety as well as for disease severity, pruritis intensity, and quality of life measures including sleep quality. C001 Study: A sustained efficacy lasted 6-8 weeks following a single IV dose 7.5 mg/kg in patients with AD appears to support C_{eff} of 5-8 μg/mL identified in a NHP IL-31 challenged model. Clinical studies in AD/PN (IL-31 driven diseases) further validate C_{eff} of 5-8 μg/mL.

FIG. 7 demonstrates simulated PK profiles. Simulation was performed with a preliminary target-mediated drug disposition (TMDD) population PK model developed with available clinical PK data from healthy subjects, and prurigo nodularis (PN) or atopic dermatitis (AD).patients.

FIG. 8 provides the study design of the Phase 2 Study to evaluate efficacy, safety, and PK in idiopathic pulmonary fibrosis and systemic sclerosis-interstitial lung disease.

FIGS. 9A-9C demonstrate that IL-6 dependent CD64+ macrophage populations drive fibrotic disease. FIG. 9A provides total hydroxyproline (Total OHP, ug/half lung) lung tissue, as measured at day 24. 4-8 mice per group. P value calculated by t-test. Graphs show mean±SD. *P<0.05. FIG. 9B provides gene expression measured in lung tissue by qRT-PCR. 4-8 mice per group. P value calculated by t-test. Graphs show mean±SD. * P<0.05. FIG. 9C provides FACS analysis of lung tissue at day 8 and day 24 from saline or BLM treated WT (Il6r+/+) and IL-6-deficient (Il6r−/−) mice showing representative FACS plots (top) and numbers of CD64+ macrophages (CD45+CD11c+SigF+MHCII+CD11b+CD64+) per 105 CD45+ cells (bottom). 4-8 mice per group. P value calculated by t-test. Graphs show mean±SD. *P<0.05.

FIG. 10 demonstrates that IL-6 activates myeloid cells and drives inflammatory and fibrotic programs. Monocyte derived macrophages (MDMs) were generated from healthy donors and polarized with IL-4 and IL-13 +/− IL-6 for 24 hours. CCL18 mRNA and protein was measured using qRT-PCR and ELISA, respectively (n=7). Graphs show mean±SD. *P<0.05.

FIG. 11 provides transcription analysis of lung biopsies obtained from healthy controls (n=9) and IPF patients (n=22). RNA-seq was carried out and CD64, CCL2 and CCL18 transcripts analyzed. P values calculated by paired t-tests or by Mann-Whitney. Graphs show mean±SD. *P<0.05.

FIG. 12 provides transcript analysis of skin biopsies obtained from healthy controls (n=20) and SSc patients (n=78). CD64, CCL2 and CCL18 transcripts measured. P values calculated by paired t-tests or by Mann-Whitney. Graphs show mean±SD. *P<0.05.

FIG. 13 provides transcript analysis of skin biopsies obtained from healthy controls (n=20) and SSc patients (n=78). CD64, CCL2 and CCL18 transcripts measured at baseline and after 24 weeks of PBO (n=44) or TCZ (n=40) treatment. P values calculated by paired t-tests or by Mann-Whitney. Graphs show mean±SD. * P<0.05.

FIG. 14A demonstrates RNA-seq results of OSM transcripts from lung biopsies were obtained from healthy controls (n=9) and IPF patients (n=22). FIG. 14B demonstrates RNA-seq results of OSM transcripts from skin biopsies obtained from healthy controls (n=20) and SSc patients (n=78).

FIG. 15A provides the results of cultured primary human SAEC, ENDO or FIB stimulated with recombinant human OSM. FIG. 15B provides the results of a comparative transcriptional analysis of cultured primary human SAEC, ENDO or FIB following 24 hrs of exposure to OSM.

FIGS. 16A-16D demonstrate that OSM mediates disease-relevant pathogenic responses in epithelial, endothelial cells and fibroblasts in an OSMR-dependent manner. FIG. 16A: Primary human SAEC, ENDO or FIB were cultured and stimulated with rhOSM (10 ng/ml) for 15 mins. Cells were either treated with anti-OSMR (50 ug/ml) or anti-OSM (10 ug/ml) from 120 mins prior to OSM treatment. Cell lysates were recovered and pSTAT3^{Tyr705} was measured by MSD. P value calculated by t-test. Graphs show mean±SD. * P<0.05. FIG. 16B: Normal primary human lung FIB (NH-LF) or IPF-derived human lung FIB (IPF-LF) were cultured and stimulated with rhOSM (10 ng/ml). Some cells were treated with anti-OSMR, at the indicated concentrations, from 120 mins prior to OSM treatment. Cell lysates were recovered and pSTAT3^{Tyr705} was measured by MSD. Data are expressed as % of remaining pSTAT3^{Tyr705} FIG. 16C: Primary human ENDO cells were cultured and stimulated with rhOSM (10 ng/ml). Some cells were treated with anti-OSMR, at the indicated concentrations, from 120 mins prior to OSM treatment. Cell lysates were recovered and pSTAT3^{Tyr705} was measured by MSD. Data is expressed as % of remaining pSTAT3^{Tyr705}. FIG. 16D: Primary human ENDO cells were cultured and stimulated with rhOSM (10 ng/ml) for 15 mins. Cells were either treated with anti-OSMR (50 ug/ml) or anti-LIFR (50 ug/ml) from 120 mins prior to OSM treatment. Cell lysates were recovered and pSTAT3^{Tyr705} was measured by MSD. P value calculated by t-test. Graphs show mean±SD. * P<0.05.

FIG. 17 demonstrates that OSM-induced endothelial cell disruption and permeability could be completely prevented with anti-OSMR antagonism. Primary human ENDO cells were cultured, and permeability was assessed following rhOSM (10 ng/ml) treatment. Cells were either treated with anti-OSMR (50 ug/ml) or anti-LIFR (50 ug/ml) from 120 mins prior to OSM treatment. P value calculated by t-test. Graphs show mean±SD. *P<0.05.

FIG. 18 demonstrates that OSM-induced IL-6 and CCL2/MCP1 secretion from lung endothelial cells was largely dependent upon OSMR rather than LIFR. Primary human ENDO cells were cultured and stimulated with rhOSM (10 ng/ml) for 24 hrs with anti-OSMR (50 ug/ml) or anti-LIFR (50 ug/ml) from 120 mins prior to OSM treatment and throughout. IL-6 and CCL2/MCP1 were measured by Luminex® in supernatants. P value calculated by t-test. Graphs show mean±SD. *P<0.05.

FIG. 19 demonstrates that OSM disrupts SAEC integrity with a significant increase in permeability. Primary human SAEC were cultured, and permeability was assessed following rhOSM (10 ng/ml) treatment. Cells were either treated with anti-OSMR (50 ug/ml) or anti-LIFR (50 ug/ml) from 120 mins prior to OSM treatment. P value calculated by t-test. Graphs show mean±SD. *P<0.05.

FIG. 20 demonstrates that OSM induced collagen secretion from primary human fibroblasts in an OSMR-dependent manner. Primary human FIB cells were cultured and stimulated with rhOSM (10 ng/ml) for 72 hrs. Cells were treated with anti-OSMR (50 ug/ml) or anti-LIFR (50 ug/ml) from 120 mins prior to OSM treatment. Collagen (COL) secretion was stained and assessed using CellInsight CX7, in the scar-in-a-jar assay. P value calculated by t-test. Graphs show mean±SD. *P<0.05.

FIG. 21 demonstrates that OSM-driven chemokine production from PCLS depends upon OSMR, not LIFR. Precision cut lung slices (PCLS) were prepared and stimulated with rhOSM (10 ng/ml) for 24 hrs with anti-OSMR (50 ug/ml) or anti-LIFR (50 ug/ml) from 120 mins prior to OSM treatment and throughout. CCL3 and CCL4 were measured by Luminex® in supernatants. P value calculated by t-test. Graphs show mean±SD. *P<0.05.

FIG. 22A shows the changes in body weight of WT C57BL/6J mice given intratracheal saline (PBS) or bleomycin (BLM) on day 0, 2 and 4. Mice were given isotype control antibody, anti-OSM mAb+isotype, anti-IL-6R mAb+isotype, or anti-IL-6R+anti-OSMR mAb (500 ug/mouse every 3 days, from day-1). Body weight was monitored weekly. FIG. 22B shows the survival of WT C57BL/6J mice given intratracheal saline (PBS) or bleomycin (BLM) on day 0, 2 and 4. Mice were given isotype control antibody, anti-OSM mAb+isotype, anti-IL-6R mAb+isotype, or anti-IL-6R+anti-OSMR mAb (500 ug/mouse every 3 days, from day −1). Mice with >25% weight loss were euthanized. FIG. 22C provides tissue volume (TV) at day 22 of WT C57BL/6J mice given intratracheal saline (PBS) or bleomycin (BLM) on day 0, 2 and 4. Mice were given isotype control antibody, anti-OSM mAb+isotype, anti-IL-6R mAb+isotype, or anti-IL-6R+anti-OSMR mAb (500 ug/mouse every 3 days, from day-1). 5-25 mice per group. P value calculated by t-test. Graphs show mean±SD. *P<0.05.

FIG. 23A shows new hydroxyproline (New OHP, ug/half lung) in lung tissue measured in deuterated water treated mice at day 24. 5-25 mice per group. Lung pathology (fibrosis score) was assessed in a blinded manner. Lung tissue was recovered at day 24 for sectioning and assessment of pathology. FIG. 23B provides representative masons trichrome stained sections shown. 5-25 mice per group. P value calculated by t-test. Graphs show individual mice and mean±SD. *P<0.05.

FIG. 24 provides total and differential cell counts (Macrophages, Mac; Lymphocytes, Lym; Neutrophils, Neut) from mice that received bronchoalveolar lavage (BAL). 5-25 mice per group. P value calculated by t-test. Graphs show mean±SD. *P<0.05.

### DETAILED DESCRIPTION

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA, and related fields as are within the skill of the art. These techniques are fully explained in the literature.

The term “herein” means the entire application.

It should be understood that any of the embodiments described herein, including those described under different aspects of the disclosure and different parts of the specification (including embodiments described only in the Examples) can be combined with one or more other embodiments disclosed herein, unless explicitly disclaimed or improper. Combination of embodiments are not limited to those specific combinations claimed via the multiple dependent claims.

Any publications, patents and published patent applications referred to in this application are specifically incorporated by reference herein. In case of conflict, the present specification, including its specific definitions, will control.

Throughout this specification, the word “comprise” or variations such as “comprises” or “comprising,” which is synonymous with “including,” “containing,” or “characterized by,” is inclusive or open-ended and does not exclude additional, unrecited elements or method steps.

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “consisting of” excludes any element, step, or ingredient not specifically recited.

The term “consisting essentially of” limits the scope of a disclosure to the specified materials or steps and those that do not materially affect the basic and novel characteristic(s) of the disclosure.

Any example(s) following the term “e.g.” or “for example” is not meant to be exhaustive or limiting.

The articles “a,” “an” and “the” are used herein to refer to one or to more than one (i.e., to at least one) of the grammatical object of the article. By way of example, “an element” means one element or more than one element.

The term “or” as used herein should be understood to mean “and/or,” unless the context clearly indicates otherwise.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10. The disclosure of a range should also be considered as disclosure of the endpoints of that range.

Exemplary methods and materials are described herein, although methods and materials similar or equivalent to those described herein can also be used in the practice or testing of the present application. The materials, methods, and examples are illustrative only and not intended to be limiting.

### Definitions

The following terms, unless otherwise indicated, shall be understood to have the following meanings:

As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Reference to “about” a value or parameter herein includes (and describes) embodiments that are directed to that value or parameter per se. For example, description referring to “about X” includes description of “X.” Numeric ranges are inclusive of the numbers defining the range.

As used herein, the term “biomarker” refers to an indicator of, for example, a pathological state of a subject, which can be detected in a biological sample of the subject. Biomarkers include DNA-based, RNA-based, and protein-based molecular markers.

As used herein, the term “diagnosis” refers to the identification or classification of a molecular or pathological state, disease, or condition. For example, “diagnosis” can refer to identification of a particular type of a condition (such as idiopathic pulmonary fibrosis or usual interstitial pneumonia (“UIP”)). “Diagnosis” can also refer to the classification of a particular subtype of a condition (such as idiopathic pulmonary fibrosis), e.g., by histopathological or radiographic criteria or by molecular features (e.g., a subtype characterized by expression of one or a combination of particular genes or proteins encoded by the genes). The term “suffering from” or “experiencing” as used herein can refer to a subject who has not received a formal diagnosis of a disease or disorder but displays several of the symptoms which may lead to a formal diagnosis of the disease or disorder.

As used herein, the term “aiding diagnosis” refers to methods that assist in making a clinical determination regarding the presence, or nature, of a particular type of symptom or condition of a condition (such as idiopathic pulmonary fibrosis). For example, a method of aiding diagnosis of a condition (such as idiopathic pulmonary fibrosis) can include measuring the expression of certain genes in a biological sample from an individual.

As used herein, the term “prognosis” is used herein to refer to the prediction of the likelihood of survival over time as well as one or more disease symptoms attributable to a condition (such as idiopathic pulmonary fibrosis) worsening over time.

As used herein, the term “initial” or “loading” dose generally comprises an initial dose of a therapeutic agent administered to a patient or subject and is followed by one or more maintenance dose(s) thereof. Generally, a single loading dose is administered, but multiple loading doses are contemplated herein. Usually, the amount of loading dose(s) administered exceeds the amount of the maintenance dose(s) administered.

As used herein, the term “maintenance” dose herein refers to one or more doses of a therapeutic agent administered to the patient over a treatment period. Usually, the maintenance doses are administered at spaced treatment intervals, such as approximately every week, approximately every 2 weeks, approximately every 3 weeks, or approximately every 4 weeks, preferably every 3 weeks. An exemplary maintenance dose for subcutaneous vixarelimab for is 360 mg.

As used herein, the term “sample” refers to a composition that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. A “tissue” or “cell sample” refers to a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample can also be primary or cultured cells or cell lines. Optionally, the tissue or cell sample is obtained from a disease tissue/organ. The tissue sample can contain compounds which are not naturally intermixed with the tissue in nature such as preservatives, anticoagulants, buffers, fixatives, nutrients, antibiotics, and the like.

As used herein, the terms “control,” “control cohort,” “reference sample,” “reference cell,” “reference tissue,” “control sample,” “control cell,” and “control tissue” refer to a sample, cell or tissue obtained from a source that is known, or believed, to not be afflicted with the disease or condition for which a method or composition of the disclosure is being used to identify. The control can include one control or multiple controls. In one embodiment, a reference sample, reference cell, reference tissue, control sample, control cell, or control tissue is obtained from a healthy part of the body of the same subject or patient in whom a disease or condition is being identified using a composition or method of the disclosure. In one embodiment, a reference sample, reference cell, reference tissue, control sample, control cell, or control tissue is obtained from a healthy part of the body of an individual who is not the subject or patient in whom a disease or condition is being identified using a composition or method of the disclosure.

The terms “amino acid” and “amino acid identity,” as used herein, refer to one of the 20 naturally occurring amino acids that are coded for by DNA and RNA.

The term “amino acid substitution” or “substitution,” as used herein, refers to the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not considered an amino acid substitution.

The terms “amino acid insertion,” “amino acid addition” or “addition” or “insertion,” as used herein, refer to the addition of an amino acid sequence at a particular position in a parent polypeptide sequence. For example, −233E, _233E or 233E designates an insertion of glutamic acid after position 233 and before position 234. Additionally, −233ADE, _233ADE or 233ADE designates an insertion of AlaAspGlu after position 233 and before position 234.

The term “amino acid deletion” or “deletion,” as used herein, refers to the removal of an amino acid sequence at a particular position in a parent polypeptide sequence. For example, E233- or E233 #, E233 ( ), E233_ or E233del designates a deletion of glutamic acid at position 233. Additionally, EDA233-, EDA233_ or EDA233 #designates a deletion of the sequence GluAspAla that begins at position 233.

As used herein, the term “antibody” or “Ab” refers to an immunoglobulin molecule (e.g., complete antibodies, antibody fragment or modified antibodies) capable of recognizing and binding to a specific target or antigen, such as a carbohydrate, polynucleotide, lipid, polypeptide, etc., through at least one antigen recognition site, located in the variable region of the immunoglobulin molecule. As used herein, the term “antibody” is used in the broadest sense and encompasses various antibody structures, including but not limited to monoclonal antibodies, polyclonal antibodies, human antibodies, engineered antibodies (including humanized antibodies, fully human antibodies, chimeric antibodies, single-chain antibodies, artificially selected antibodies, CDR-granted antibodies, etc.), monospecific antibodies and multi-specific antibodies (e.g., bispecific antibodies, wherein the bispecific antibodies each have at least two binding sites and specifically bind two different antigens or the same antigen at two different epitopes), and antibody fragments that retain the desired antigen-binding activity. In some embodiments, “antibody” and/or “immunoglobulin” (Ig) refers to a polypeptide comprising at least two heavy (H) chains (about 50-70 kDa) and two light (L) chains (about 25 kDa), optionally inter-connected by disulfide bonds. In some embodiments, the antibody is a full-length antibody. There are two types of light chain: λ and κ. In humans, λ and κ light chains are similar, but only one type is present in each antibody. Heavy chains are classified as mu, delta, gamma, alpha, or epsilon, and define the antibody's isotype as IgM, IgD, IgG, IgA, and IgE, respectively. See generally, Fundamental Immunology Ch. 7 (Paul, W., ed., 2^{nd} ed. Raven Press, N.Y. (1989)). The methods, uses, and compositions-for-use disclosed herein utilize IgG antibodies.

The terms “nucleic acid,” “polynucleotide” and “oligonucleotide” are used interchangeably and refer to a deoxyribonucleotide or ribonucleotide polymer, in linear or circular conformation, and in either single-or double-stranded form. For the purposes of the present disclosure, these terms are not to be construed as limiting with respect to the length of a polymer.

The term “complementarity determining region” or “CDR” as used herein refers to each of the regions of an antibody variable domain which are hypervariable in sequence and which determine antigen binding specificity, for example “hypervariable regions” or (“HVRs”).

Generally, monospecific antibodies comprise six CDRs: three in the VH (H1, H2, H3), and three in the VL (L1, L2, L3). Multispecific antibodies typically comprise multiple sets of six CDRs. For example, a bispecific antibody generally comprises at least two sets of six CDRs. Exemplary CDRs herein include:
(a) hypervariable loops occurring at amino acid residues 26 32 (L1), 50 52 (L2), 91 96 (L3), 26 32 (H1), 53 55 (H2), and 96 101 (H3) (Chothia and Lesk, J. Mol. Biol. 196:901 917 (1987));
(b) CDRs occurring at amino acid residues 24 34 (L1), 50 56 (L2), 89 97 (L3), 31 35b (H1), 50 65 (H2), and 95 102 (H3) (Kabat et al., Sequences of Proteins of Immunological Interest, 5^{th} Ed. Public Health Service, National Institutes of Health, Bethesda, MD (1991)); and
(c) antigen contacts occurring at amino acid residues 27c 36 (L1), 46 55 (L2), 89 96 (L3), 30 35b (H1), 47 58 (H2), and 93 101 (H3) (MacCallum et al. J. Mol. Biol. 262:732 745 (1996)).

The term “subject” is used interchangeably herein with “patient” to refer to an individual to be treated. The subject is a mammal (e.g., human, non-human primate, rat, mouse, cow, horse, pig, sheep, goat, dog, cat, etc.). The subject can be a clinical patient, a clinical trial volunteer, an experimental animal, etc. The subject can be suspected of having or at risk for having a condition (such as idiopathic pulmonary fibrosis) or be diagnosed with a condition (such as idiopathic pulmonary fibrosis). The subject can also be suspected of having or at risk for having a lung disease or be diagnosed with a lung disease such as, for example, hypersensitivity pneumonitis, cryptogenic organizing pneumonia, diffuse alveolar damage, chronic obstructive pulmonary disease, chronic bronchitis, pulmonary emphysema, pulmonary arterial hypertension, nonspecific interstitial pneumonitis, systemic sclerosis associated interstitial lung disease, or collagen vascular disease-associated interstitial lung disease. In some embodiments, the subject to be treated according to this disclosure is a human.

As used herein, “treating,” “treatment” and “alleviation” refer to measures, wherein the object is to prevent or slow down (lessen) the targeted pathologic condition or disorder or relieve some of the symptoms of the disorder. Those in need of treatment can include those already with the disorder as well as those prone to have the disorder, those at risk for having the disorder and those in whom the disorder is to be prevented. In some embodiments, the subject in need of treatment already has the disorder. For example, a subject is successfully “treated” for idiopathic pulmonary fibrosis or systemic sclerosis associated interstitial lung disease if, after receiving a therapeutic agent, the subject shows observable and/or measurable decrease or change from baseline in and/or measurable rate of change from baseline over time (e.g., over 3 months (12 weeks), or 6 months (24 weeks), or 9 months (36 weeks), or 12 months (1 year, 52 weeks) in one or more of the following: forced vital capacity (FVC), diffusion capacity of the lung for carbon monoxide (DLco), a subject reported outcome tool, such as A Tool to Assess Quality of Life in idiopathic pulmonary fibrosis (ATAQ-IPF) or EuroQol 5-Dimension Questionnaire (EQ-5D), St. George's Respiratory Questionnaire (SRGQ), 6-minute walk distance (6MWD), resting oxygen flow rate, radiographic findings on pulmonary high-resolution computed tomography (HRCT), including quantitative lung fibrosis (QLF) score, serum biomarkers including CXCL14, periostin, CCL18 (Chemokine (C-C motif) ligand 18), YKL40 (chitinase-3-like protein; CHI3L1), COMP (cartilage oligomeric matrix protein), OPN (osteopontin), CCL13 (Chemokine (C-C motif) ligand 13).

“Administering” or “administration of” a substance, a compound or an agent to a subject refers to the contact of that substance, compound or agent to the subject or a cell, tissue, organ, or bodily fluid of the subject. For example, a compound or an agent can be administered intravenously or subcutaneously. In some embodiments, a “combination” or a “combination therapy” refers to the administration of more than one therapeutic agent. When more than one substance, compound or agent is being administered, the administration can be simultaneous or sequential. “Simultaneous administration” refers to the administration of multiple therapeutic agents at the same time. The simultaneously administered therapeutic agents can be co-formulated or mixed prior to administration. “Sequential administration” refers to the administration of multiple therapeutic agents at different times in a manner that achieves overlapping results. For example, two therapeutic agents can be administered on same day in two separate injections. As an alternate example, one of the agents can be injected on one day, and the second can be injected on a subsequent day. Sequential administration is not limited to instances in which more than one therapeutic agent is present in the subject's body. For example, if a first therapeutic agent expands the subject's T cell population, and a second therapeutic agent targets the subject's T cells to a tumor, then the two agents can be sequentially administered if the second agent is administered at a time when the subject's T cell population is still expanded, even if none of the first therapeutic agent remains in the subject's body. Administering can also be performed, for example, once, a plurality of times, and/or over one or more extended periods. The administration may be either direct administration, including self-administration, or indirect administration, including the act of prescribing a drug. For example, as used herein, a physician who instructs a subject to self-administer a drug, or to have the drug administered by another and/or who provides a subject with a prescription for a drug is administering the drug to the subject.

An “effective amount” or refers to an amount effective, at dosages and for periods of time necessary, to achieve the desired therapeutic or prophylactic result. The term “therapeutically effective amount” refers to an amount effective to “alleviate” or “treat” a disease or disorder in a subject. A therapeutically effective amount of a therapeutic agent can vary according to factors such as the disease state, age, gender, and weight of the individual, and the ability of the antibody to elicit a desired response in the subject. A therapeutically effective amount is also one in which any toxic or detrimental effects of the therapeutic agent are outweighed by the therapeutically beneficial effects. A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount. “Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

As used herein, the terms “polypeptide,” “peptide” and “protein” are used interchangeably to refer to a polymer of amino acid residues. Expression of an antibody in a cell can result from delivery of the antibody protein to the cell or by delivery of a polynucleotide encoding the antibody to a cell, wherein the polynucleotide is transcribed, and the transcript is translated, to generate the antibody. Trans-splicing, polypeptide cleavage and polypeptide ligation can also be involved in expression of a protein in a cell. Methods for polynucleotide and polypeptide delivery to cells are known in the art.

The term “package insert” is used to refer to instructions customarily included in commercial packages of therapeutic products that contain information about the indications, usage, dosage, administration, combination therapy, contraindications and/or warnings concerning the use of such therapeutic products.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “OSM” as used herein refers to oncostatin M. The term “IL-31” as used herein refers to interleukin-31. Both OSM and IL-31 are well-known cytokines which are members of the IL-6 superfamily. The term “OSMR” as used herein refers to the oncostatin M receptor and is also referred to herein as “OSMRβ” or “OSMR Type II.” OSM is a member of the type I cytokine receptor family. OSMRβ heterodimerizes with glycoprotein 130 (also known herein as gp130) to form the type II OSMR which transduces OSM-induced signaling events. OSMRβ also heterodimerizes with IL-31 receptor A (IL31RA) to form the IL-31 receptor which transduces IL-31-induced signaling events. An exemplary human OSMRβ amino acid sequence is provided in GenBank Accession No. NP_003990.

As used herein, “IL-6,” “IL6” or “Interleukin 6” may be used interchangeably and refer to a four-α-helix protein belonging to a family of cytokines. IL-6 acts as both a proinflammatory cytokine and an anti-inflammatory myokine.

As used herein, “tocilizumab” refers to a recombinant humanized monoclonal antibody that binds to human interleukin-6 receptor (IL-6R), listed in the International Nonproprietary Names for Pharmaceutical Substances (INN) Proposed List 90 (WHO Drug Information, Vol. 18, No. 1, 2004, p. 66) and having the heavy and light chain amino acid sequences listed under CAS Registry Number 375823-41-9. It is an IgG1κ (gamma 1, kappa) antibody with two heavy chains and two light chains forming two antigen-binding sites. In a preferred embodiment, the heavy chain and light chain amino acid sequences of tocilizumab comprise SEQ ID NOs.: 13 and 14, respectively. Tocilizumab is also known in the art as “Actemra®” or “RoActemra®.”

As used herein, the term “vixarelimab” refers to a monoclonal antibody that targets oncostatin M receptor beta (OSMRβ), which mediates signaling of interleukin-31 (IL-31) and oncostatin M (OSM) protein having the heavy and light chain amino acid sequences listed in the International Nonproprietary Names for Pharmaceutical Substances (INN) List 85 (WHO Drug Information, Vol. 35, No. 1, 2021, pp. 228-229). In some embodiments, vixarelimab comprises a heavy chain having the amino acid sequence of SEQ ID NO: 1 and a light chain having the amino acid sequence of SEQ ID NO: 2. Vixarelimab is also known in some non-patent publications as “KPL-716.”

### General

Two prevailing unmet needs have emerged in the treatment of interstitial lung diseases (ILDs), including idiopathic pulmonary fibrosis (IPF) and systemic sclerosis with ILD (SSc-ILD): preserving lung function and stalling progressive fibrosis. Tocilizumab (an anti-IL6R antibody) was recently approved for the treatment of SSc-ILD. While tocilizumab has been shown to prevent lung function decline, there remains a growing need to identify and develop therapeutics to stall progressive fibrosis.

IL-11 has recently emerged as an important contributor to lung, liver, and cardiac fibrosis. See, e.g., Schafer, S., et al. IL-11 is a crucial determinant of cardiovascular fibrosis. Nature 552, 110-115 (2017); Ng, B., et al. Interleukin-11 is a therapeutic target in idiopathic pulmonary fibrosis. Sci Transl Med 11(2019); and Effenberger, M., et al. Interleukin-11 drives human and mouse alcohol-related liver disease. Gut 72, 168-179 (2023). Accordingly, the inventors considered IL-11 as a potential target for stalling progressive fibrosis. However, while IL-11 was observed to contribute to airway inflammation in vivo (data not shown) and to induce inflammatory cytokine responses in lung fibroblasts in vitro (data not shown), IL-11 did not appear to have fibrogenic properties in the lung in vivo or with lung-derived fibroblasts in vitro (data not shown).

Oncostatin M (OSM) has both positively and negatively been associated with fibrosis. Surprisingly, however, the requirement for OSM and OSMR signaling in pulmonary fibrosis does not appear to have been tested in the art, genetically or pharmacologically. As described in more detail in the Examples below, OSM orchestrated lung injury response contributing to epithelial and endothelial cell disruption, myofibroblast activation and fibrosis. In humans OSM binds to gp130 and heterodimerizes with one of two receptors, OSMR or LIFR, for signal transduction. As demonstrated in the Examples below, of the two receptors, it is OSMR, not LIFR, that acts as the dominant receptor complex used by OSM in a disease-relevant context. Further, OMSR antagonism alone was nearly sufficient to mitigate OSM-driven pSTAT3 phosphorylation in fibroblasts and epithelial cells. The roles of OSM and IL-6 in ILD were shown herein to be non-overlapping. Accordingly, the experiments and data presented herein demonstrate that inhibition of OSM/OSMRβ pathway may be used to stall progressive fibrosis in ILD patients.

### Vixarelimab and Inhibition of the OSM/OSMRβ Pathway

### Vixarelimab

Vixarelimab is a monoclonal antibody that targets OSMRβ (oncostatin M (OSM) receptor) (described as “Ab2” in U.S. Pat. No. 9,593,163 (herein, the “163 patent”), the contents of which are incorporated herein by reference in their entirety). OSMRβ is a cytokine receptor subunit that heterodimerizes with IL-31 receptor alpha (IL-31Rα) or gp130 to form two distinct receptors for two distinct cytokines, interleukin-31 (IL-31) and OSM, respectively, each of them mediating signaling pathways implicated in inflammation and fibrosis (Mozaffarian et al. 2008;Marden et al. 2020; Yaseen et al. 2020; Kuzumi et al. 2021).

Vixarelimab is one of 3 anti-OSMRβ antibodies generated and described in the '163patent which were shown using in vitro cellular assays to block signaling through human OSMRβ. The assays, described at least in Examples 2 and 3 of the '163 patent, demonstrate that the three anti-OSMRβ antibodies (“Ab1,” “Ab2,” and “Ab3”) were each a potent inhibitor of both OSM-mediated and of IL-31-mediated signaling. The heavy and light chain sequences are provided in Table 1 below. In preferred embodiments, the preferred anti-OSMRβ antibody for treatment of pulmonary fibrotic disorders as described herein is vixarelimab.

TABLE 1

| | SEQ ID | |
|---|---|---|
| | NO | Sequence |
| Ab1 Heavy | 1 | QVQLVQSGAEVKKPGASVKVSCKASGYTFTSYDINWVRQA |
| chain | | TGQGLEWMGWMNPNSGNTDYAQKFQGRVTMTRNISISTA |
| | | YIELSSLRSEDTAVYYCARDMVAANTDYYFYYGMDVWGQ |
| | | GTTVTVSSASTKGPSVFPLAPCSRSTSESTAALGCLVKDYFP |
| | | EPVTVSWNSGALTSGVHTFPAVLQSSGLYSLSSVVTVPSSNF |
| | | GTQTYTCNVDHKPSNTKVDKTVERKCCVECPPCPAPPVAGP |
| | | SVFLFPPKPKDTLMISRTPEVTCVVVDVSHEDPEVQFNWYV |
| | | DGVEVHNAKTKPREEQFNSTFRVVSVLTVVHQDWLNGKEY |
| | | KCKVSNKGLPAPIEKTISKTKGQPREPQVYTLPPSREEMTKN |
| | | QVSLTCLVKGFYPSDIAVEWESNGQPENNYKTTPPMLDSDG |
| | | SFFLYSKLTVDKSRWQQGNVFSCSVMHEALHNHYTQKSLS |
| | | LSPGK |
| Ab1 Light | 2 | QSVLTQPPSASGTPGQRVTISCSGSSSNVGSNTVSWYQQLP |
| chain | | GTAPKLLIYTNNRRPSGVPDRFSGSKSGTSASLAISGLQ |
| | | SEDEADYFCAALDDSLNGVVFGGGTKLTVLGQPKAAPSVT |
| | | LFPPSSEELQANKATLVCLISDFYPGAVTVAWKADSSPVK |
| | | AGVETTTPSKQSNNKYAASSYLSLTPEQWKSHRSYSCQVTH |
| | | EGSTVEKTVAPTECS |
| Ab1 heavy | 3 | QVQLVQSGAEVKKPGASVKVSCKASGYTFTSYDINWVRQA |
| chain | | TGQGLEWMGWMNPNSGNTDYAQKFQGRVTMTRNISISTA |
| variable | | YIELSSLRSEDTAVYYCARDMVAANTDYYFYYGMDVWGQ |
| domain | | GTTVTVSS |
| Ab1 Light | 4 | QSVLTQPPSASGTPGQRVTISCSGSSSNVGSNTVSWYQQL |
| chain | | PGTAPKLLIYTNNRRPSGVPDRFSGSKSGTSASLAISGLQ |
| variable | | SEDEADYFCAALDDSLNGVVFGGGTKLTVLG |
| domain | | |
| Vixarelimab | 5 | QVQLVQSGAEVKKPGASVKVSCKASGYTFTSYEINWVRQA |
| Heavy chain | | TGQGLEWMGWMNPNSGYTGYAQKFQGRVTMTRDTSISTA |
| (“Ab2”) | | YMEMSSLRSEDTAVYYCARDIVAANTDYYFYYGMDVWGQ |
| | | GTTVTVSSASTKGPSVFPLAPCSRSTSESTAALGCLVKDYFP |
| | | EPVTVSWNSGALTSGVHTFPAVLQSSGLYSLSSVVTVPSSSL |
| | | GTKTYTCNVDHKPSNTKVDKRVESKYGPPCPPCPAPEFLGG |
| | | PSVFLFPPKPKDTLMISRTPEVTCVVVDVSQEDPEVQFNWY |
| | | VDGVEVHNAKTKPREEQFQSTYRVVSVLTVLHQDWLNGK |
| | | EYKCKVSNKGLPSSIEKTISKAKGQPREPQVYTLPPSRDELT |
| | | KNQVSLTCLVKGFYPSDIAVEWESNGQPENNYKTTPPVLDS |
| | | DGSFFLYSKLTVDKSRWQQGNVFSCSVMHEALHNHYTQKS |
| | | LSLSPG |
| Vixarelimab | 6 | QSVLTQPPSASGTPGQRVTISCSGSNSNIGSNTVNWYHQLPG |
| Light chain | | TAPKLLIYNINKRPSGVPDRFSGSKSGSSASLAISGLQSEDE |
| (“Ab2”) | | ADYYCSTWDDSLDGVVFGGGTKLTVLGQPKAAPSVTLFPPSS |
| | | EELQANKATLVCLISDFYPGAVTVAWKADSSPVKAGVETTT |
| | | PSKQSNNKYAASSYLSLTPEQWKSHRSYSCQVTHEGSTVEK |
| | | TVAPTECS |
| Vixarelimab | 7 | QVQLVQSGAEVKKPGASVKVSCKASGYTFTSYEINWVRQA |
| heavy chain | | TGQGLEWMGWMNPNSGYTGYAQKFQGRVTMTRDTSISTA |
| variable | | YMEMSSLRSEDTAVYYCARDIVAANTDYYFYYGMDVWGQ |
| domain | | GTTVTVSS |
| (“Ab2”) | | |
| Vixarelimab | 8 | QSVLTQPPSASGTPGQRVTISCSGSNSNIGSNTVNWYHQLPG |
| Light chain | | TAPKLLIYNINKRPSGVPDRFSGSKSGSSASLAISGLQSEDE |
| variable | | ADYYCSTWDDSLDGVVFGGGTKLTVLG |
| domain | | |
| (“Ab2”) | | |
| Ab3 Heavy | 9 | QVHLVQSGAEVKKPGASVKVSCKASGYTFTSYGISWVRQA |
| chain | | PGQGLEWMGWLSTYSGNTNYAQKLQGRVTMTTDTSTSTA |
| | | YMELRSLRSDDTAVYYCARGNFYYYGMDVWGQGTTVTVS |
| | | SASTKGPSVFPLAPCSRSTSESTAALGCLVKDYFPEPVTVSW |
| | | NSGALTSGVHTFPAVLQSSGLYSLSSVVTVPSSNFGTQTYTC |
| | | NVDHKPSNTKVDKTVERKCCVECPPCPAPPVAGPSVFLFPP |
| | | KPKDTLMISRTPEVTCVVVDVSHEDPEVQFNWYVDGVEVH |
| | | NAKTKPREEQFNSTFRVVSVLTVVHQDWLNGKEYKCK |
| | | VSNKGLPAPIEKTISKTKGQPREPQVYTLPPSREEMTKNQVS |
| | | LTCLVKGFYPSDIAVEWESNGQPENNYKTTPPMLDSDGSFF |
| | | LYSKLTVDKSRWQQGNVFSCSVMHEALHNHYTQKSLSLSP |
| | | GK |
| Ab3 Light | 10 | EIVLTQSPGTLSLSPGERATLSCRASQSVSSSYLAWYQQKPG |
| chain | | QAPRLLIFGASSRATGIPDRFSGSGSGTDFTLTISRLEPEDF |
| | | AVYYCQQYGSSPPITFGQGTRLEIKRTVAAPSVFIFPPSDEQ |
| | | LKSGTASVVCLLNNFYPREAKVQWKVDNALQSGNSQESVTEQ |
| | | DSKDSTYSLSSTLTLSKADYEKHKVYACEVTHQGLSSPVTKS |
| | | FNRGEC |
| Ab3 heavy | 11 | QVHLVQSGAEVKKPGASVKVSCKASGYTFTSYGISWVRQA |
| chain | | PGQGLEWMGWLSTYSGNTNYAQKLQGRVTMTTDTSTSTA |
| variable | | YMELRSLRSDDTAVYYCARGNFYYYGMDVWGQGTTVTVS |
| domain | | S |
| Ab3 Light | 12 | EIVLTQSPGTLSLSPGERATLSCRASQSVSSSYLAWYQQKPG |
| chain | | QAPRLLIFGASSRATGIPDRFSGSGSGTDFTLTISRLEPEDF |
| variable | | AVYYCQQYGSSPPITFGQGTRLEIKR |
| domain | | |

In some embodiments, the anti-OSMRβ antibody is Ab1. In some embodiments, the heavy chain comprises the amino acid sequence of SEQ ID NO: 1. In some embodiments, the light chain comprises the amino acid sequence of SEQ ID NO: 2. In some embodiments, the heavy chain variable domain comprises the amino acid sequence of SEQ ID NO: 3. In some embodiments, the light chain variable domain comprises the amino acid sequence of SEQ ID NO: 4. In some embodiments, the heavy chain comprises the amino acid sequence of SEQ ID NO: 1 and the light chain comprises the amino acid sequence of SEQ ID NO: 2.

In some embodiments, the anti-OSMRβ antibody is vixarelimab (“Ab2”). In some embodiments, the heavy chain comprises the amino acid sequence of SEQ ID NO: 5. In some embodiments, the light chain comprises the amino acid sequence of SEQ ID NO: 6. In some embodiments, the heavy chain variable domain comprises the amino acid sequence of SEQ ID NO: 7. In some embodiments, the light chain variable domain comprises the amino acid sequence of SEQ ID NO: 8. In some embodiments, the heavy chain comprises the amino acid sequence of SEQ ID NO: 5 and the light chain comprises the amino acid sequence of SEQ ID NO: 6.

In some embodiments, the anti-OSMRβ antibody is Ab3. In some embodiments, the heavy chain comprises the amino acid sequence of SEQ ID NO: 9. In some embodiments, the light chain comprises the amino acid sequence of SEQ ID NO: 10. In some embodiments, the heavy chain variable domain comprises the amino acid sequence of SEQ ID NO:11. In some embodiments, the light chain variable domain comprises the amino acid sequence of SEQ ID NO: 12. In some embodiments, the heavy chain comprises the amino acid sequence of SEQ ID NO: 9 and the light chain comprises the amino acid sequence of SEQ ID NO: 10.

### IL-31

IL-31 is a T-cell-derived cytokine which appears to be involved in the cutaneous and epithelial signs and symptoms observed in pruritus, skin inflammation, and airway hypersensitivity (Kabashima and Irie, 2021, Front in Med, 8:638325; Dillon et al, 2004, Nat Immunol, 5:752-760). Vixarelimab was used in clinical studies in patients diagnosed with AD or PN. Phase 2 clinical studies of vixarelimab (ClinicalTrials.gov Identifiers NCT03816891 and NCT03858634) include a randomized, double blind, placebo-controlled clinical trial to evaluate the efficacy, safety, tolerability, PK, and immunogenicity of vixarelimab administered SC in subjects with prurigo nodularis (PN) experiencing pruritus. Results from these previous clinical studies of vixarelimab have produced safety and efficacy data in inflammatory, hyperkeratotic skin disorders. With respect to the role of IL-31 in pulmonary fibrotic disorders, IL-31 treatment in mice has resulted in significant fibrosis in the central lung area (Yaseen et al., 2020,Rheumatology, 59:2625-2636) while the loss of IL-31 signaling in a pulmonary fibrosis mouse model attenuated collagen deposition and lung function decline (Yombo et al., 2021, Front Immunol, 12: s645717).

### OSM

OSM is a cytokine in the IL-6 superfamily and is expressed in a variety of immune cells including activated T cells, monocytes, dendritic cells, neutrophils, activated mast cells and eosinophils (Wallace et al., 1999, J Immunol, 162:5547-5555; Stawski and Trojanowska, 2019, Connect Tissue Res, 60:40-49). OSM signaling can be initiated by binding to one of two types of OSM receptors: the type I receptor complex (LIFRb/gp130) or type II receptor complex (OSMRβ/gp130).

OSM protein is increased in bronchoalveolar lavage (BAL) samples of patients with IPF and SSc-ILD as compared with healthy controls (Mozaffarian et al. 2008). Data show that OSM mRNA is increased in the lungs of patients with IPF compared with controls (e.g., see Example 1 herein). Viral overexpression or delivery of recombinant OSM to the lungs of mice is sufficient to induce inflammation and fibrotic remodeling (Mozaffarian et al. 2008; Wong et al. 2014). Moreover, OSM enhances the survival and proliferation of lung fibroblasts and promotes the production of collagen (Scaffidi et al. 2002). Data show that inhibiting OSM using an anti-OSM antibody or genetic deletion of OSM in a bleomycin model of pulmonary fibrosis leads to a reduction in lung injury and collagen deposition (e.g., see Example 2 herein).

In addition, interstitial lung disease (ILD) is a common manifestation of systemic sclerosis (SSc), an autoimmune disorder characterized by the fibrosis of skin and other organ systems. Lung fibrosis present in SSc patients is a key prognostic indicator for SSc patients and the most common cause of death in these patients. OSM is up-regulated in the CD8+ T cells in the lungs of SSc patients with ILD relative to patients without lung disease or healthy controls (Luzina et al., 2003, Arthritis Rheum, 48:2262-2274).

### Vixarelimab for Treatment of Pulmonary Fibrotic Disorders

While prior clinical studies of vixarelimab in patients suffering from skin disorders such as PN have shown both safety and some efficacy, there are no clinical data describing treatment of pulmonary fibrosis patients with an anti-OSMRβ antibody such as vixarelimab, which blocks both OSM and IL-31 signaling. Importantly, dosing of the antibody to achieve therapeutic activity in patients suffering from fibrotic pulmonary disorders remains a challenge considering the unknown impact of anti-OSMRβ antibody exposure to OSMRβ on the surface of cells in the pulmonary vs. skin environment. Indeed, translation from IL-31-driven pruritic indications to OSM-driven fibrosis is particularly unpredictable.

As described herein, PK/PD modeling (e.g., Dua et al., 2014, CPT Pharmacometrics Syst. Pharmacol, 4:324-337) was used to predict a therapeutically effective dose of an anti-OSMRβ antibody for treatment of pulmonary fibrotic disorders including but not limited to IPF and SSc-ILD. The modeling relied in part on in vitro potency assays, preclinical PK/PD studies, and PK data from doses tested in phase 1 and 2 trials with AD and PN patients (see, e.g., Example 3 herein). The present disclosure provides methods for treating pulmonary fibrotic disorders by administering to a subject in need thereof an anti-OSMRβ wherein the anti-OSMRβ antibody binds the extracellular domain of the OSMRβ protein and blocks signaling of the type II OSMR by both OSM and IL31. In preferred embodiments, the dosing regimen for administering the vixarelimab antibody is 360 mg every 2 weeks. In some embodiments, the patient does not receive a loading dose.

Also, without being bound by theory, it is considered that blocking OSM activation of the type II receptor without inhibiting the type I OSMRβ receptor may provide a better safety profile in patients receiving the anti-OSMRβ antibody as described herein. For example, binding of the therapeutic antibody to the OSMRβ subunit of the type II receptor allows continued signaling of OSM through the type I receptor. In some embodiments, administration of an anti-OSMRβ antibody to a subject suffering from a fibrotic disease does not elicit more than a mild case of anemia or does not cause unsafe elevation of thrombopoietin and/or erythropoietin.

Accordingly, the present disclosure provides methods for administering vixarelimab, or another anti-OSMRβ antibody that inhibits both OSM and IL-31 signaling, to treat a pulmonary fibrotic disorder in a patient. In some embodiments, the disorder is idiopathic pulmonary fibrosis (IPF). In some embodiments, the disorder is SSc-ILD. In some embodiments, the antibody can be administered subcutaneously.

In one aspect, a method for treating a pulmonary fibrotic disease is provided. In some embodiments, the method comprises administering to a subject in need thereof a therapeutically effective dose of an anti-OSMRβ (oncostatin M receptor beta) antibody. In another aspect, the present disclosure provides use of a therapeutically effective dose of an anti-OSMRβ (oncostatin M receptor beta) antibody in the manufacture of a medicament for the treatment of a pulmonary fibrotic disease in a subject in need thereof. In a further aspect, the present disclosure provides a therapeutically effective amount of an anti-OSMRβ antibody for use in treating a pulmonary fibrotic disease in a subject in need thereof. In some embodiments, the pulmonary fibrotic disease is selected from the group consisting of progressive pulmonary fibrosis (PPF), idiopathic pulmonary fibrosis (IPF), and systemic sclerosis-interstitial lung disease (SSc-ILD). In some embodiments, the pulmonary fibrotic disease is progressive pulmonary fibrosis (PPF). In some embodiments, the pulmonary fibrotic disease is idiopathic pulmonary fibrosis (IPF). In some embodiments, the pulmonary fibrotic disease is systemic sclerosis-interstitial lung disease (SSc-ILD).

In one aspect, a method of increasing the forced vital capacity (FVC) in a subject suffering from a pulmonary fibrotic disorder is provided. In some embodiments, the method comprises administering a therapeutically effective dose of anti-OSMRβ antibody to a subject in need thereof. In another aspect, the present disclosure provides use of a therapeutically effective dose of an anti-OSMRβ (oncostatin M receptor beta) antibody in the manufacture of a medicament for increasing the forced vital capacity (FVC) in a subject suffering from a pulmonary fibrotic disorder. In a further aspect, the present disclosure provides a therapeutically effective amount of an anti-OSMRβ antibody for use in increasing the forced vital capacity (FVC) in a subject suffering from a pulmonary fibrotic disorder.

In one aspect, a method of increasing the distance traveled, as measured the 6-minute walk test (6MWT), by a subject suffering from a pulmonary fibrotic disorder is provided. In some embodiments, the method comprises administering a therapeutically effective dose of anti-OSMRβ antibody to a subject in need thereof. In another aspect, the present disclosure provides use of a therapeutically effective dose of an anti-OSMRβ (oncostatin M receptor beta) antibody in the manufacture of a medicament for increasing the distance traveled, as measured the 6-minute walk test (6MWT), by a subject suffering from a pulmonary fibrotic disorder. In a further aspect, the present disclosure provides a therapeutically effective amount of an anti-OSMRβ antibody for use in increasing the distance traveled, as measured the 6-minute walk test (6MWT), by a subject suffering from a pulmonary fibrotic disorder.

In one aspect, a method of reducing cough frequency, as measured a digital continuous ambulatory cough detection device, by a subject suffering from a pulmonary fibrotic disorder is provided. In some embodiments, the method comprises administering a therapeutically effective dose of anti-OSMRβ antibody to a subject in need thereof. In another aspect, the present disclosure provides use of a therapeutically effective dose of an anti-OSMRβ (oncostatin M receptor beta) antibody in the manufacture of a medicament for reducing cough frequency, as measured a digital continuous ambulatory cough detection device, by a subject suffering from a pulmonary fibrotic disorder. In a further aspect, the present disclosure provides a therapeutically effective amount of an anti-OSMRβ antibody for use in reducing cough frequency, as measured a digital continuous ambulatory cough detection device, by a subject suffering from a pulmonary fibrotic disorder.

In a one aspect, a method for treating an inflammatory disease is provided. In some embodiments, the method comprises comprising administering to a subject in need thereof a therapeutically effective dose of an anti-OSMRβ (oncostatin M receptor beta) antibody. In another aspect, the present disclosure provides use of a therapeutically effective dose of an anti-OSMRβ (oncostatin M receptor beta) antibody in the manufacture of a medicament for treating an inflammatory disease in a subject in need thereof. In a further aspect, the present disclosure provides a therapeutically effective amount of an anti-OSMRβ antibody for use in treating an inflammatory disease in a subject in need thereof.

In some embodiments of any of the above aspects, the subject is human.

### Combination Therapies

According to the present disclosure, vixarelimab or other anti-OSMRß antibodies which bind OSMRβ and block IL-31 and OSM signaling can be used either alone or in combination with other agents in a therapy. For instance, an anti-OSMRβ antibody (e.g., vixarelimab) may be co-administered with at least one additional therapeutic agent.

In some embodiments, the vixarelimab or other anti-OSMRβ antibodies which bind OSMRβ and block IL-31 and OSM signaling is used in combination with an interleukin ligand or receptor antagonist. In some embodiments, the interleukin ligand or receptor antagonist is an interleukin-6 (IL-6) ligand or receptor antagonist. In some embodiments, the IL-6 ligand or receptor antagonist is an anti-IL-6 antibody. In some embodiments, the IL-6 ligand or receptor antagonist is an anti-IL-6 receptor antibody. In some embodiments, the anti-IL-6 receptor antibody is tocilizumab or sarilumab. In some embodiments, the anti-IL-6 receptor antibody is tocilizumab. In some embodiments, the anti-IL-6 receptor antibody is sarilumab.

In certain embodiments, a subject is administered an anti-OSMRβ antibody of the disclosure in combination with a therapeutic agent for the treatment of IPF. Certain therapeutic agents have been previously described as candidates or agents for the treatment of IPF. These have been described in the published literature and are reviewed, for example, in Rafli et al., J. Thorac. Dis (2013) 5(1):48-73. Such agents include agents that have antioxidant, immunosuppressant and/or anti-inflammatory activities such as N-acetylcysteine; agents that have antifibrotic, anti-inflammatory and/or antioxidant activities such as pirfenidone, an orally administered pyridine which has been approved for clinical use in the treatment of IPF; or a tyrosine kinase inhibitor such as nintedanib; or an antibody against αvβ6 integrin (e.g., STX-100); agents that inhibit connective tissue growth factor (CTGF), such as an anti-CTGF antibody (e.g., FG-3019); agents that inhibit somatostatin receptors, such as somatostatin analogs (e.g., SOM230, octreotide); agents that inhibit IL-13, IL-4 and CCL2, such as an anti-IL13 antibody (e.g., QAX576, tralokinumab, lebrikizumab), an anti-IL4 antibody, a combination anti-IL13/anti-IL4 agent (e.g., a bispecific anti-IL13/anti-IL4 antibody such as SAR156597), an anti-IL-6 inhibitor (e.g., tocilizumab, sarilumab), an anti-CCL2 antibody (e.g., CNTO888); agents that have anti-angiogenic, immunomodulatory, and/or anti-inflammatory activities such as thalidomide or minocycline; agents that inhibit the enzyme lysyl oxidase-like 2 (LOXL2), such as an anti-LOXL2 antibody (e.g., GS-6624 [simtuzumab]); agents that inhibit angiogenesis such as the tyrosine kinase inhibitor, BIBF 1120, tetrathiomolybdate; agents that inhibit deposition of extracellular matrix and/or disrupt collagen deposition, such as doxycycline; agents that target the renin-angiotensin system such as losartan; and other agents having anti-proliferative and/or anti-fibrotic activities such as carbon monoxide.

Such combination therapies noted above encompass combined administration (where two or more therapeutic agents are included in the same or separate formulations), and separate administration, in which case, administration of the anti-OSMRβ antibody of the disclosure can occur prior to, simultaneously, and/or following, administration of the additional therapeutic agent or agents. In some embodiments, administration of the anti-OSMRβ antibody (e.g., vixarelimab) and administration of an additional therapeutic agent (e.g., an anti-IL-6 inhibitor (e.g., tocilizumab, sarilumab)) occur within about one month or within about one, two or three weeks, or within about one, two, three, four, five, or six days, of each other. In some embodiments, administration of the anti-OSMRβ antibody (e.g., vixarelimab) and administration of an additional therapeutic agent (e.g., tocilizumab) occur within about one month of each other. In one embodiment, administration of the anti-OSMRβ antibody (e.g., vixarelimab) and administration of an additional therapeutic agent (e.g., tocilizumab) occur within about one week of each other. In one embodiment, administration of the anti-OSMRβ antibody (e.g., vixarelimab) and administration of an additional therapeutic agent (e.g., tocilizumab) occur within about two weeks of each other. In one embodiment, administration of the anti-OSMRβ antibody (e.g., vixarelimab) and administration of an additional therapeutic agent (e.g., tocilizumab) occur within about three weeks of each other. In one embodiment, administration of the anti-OSMRβ antibody (e.g., vixarelimab) and administration of an additional therapeutic agent (e.g., tocilizumab) occur within about one day of each other. In one embodiment, administration of the anti-OSMRβ antibody (e.g., vixarelimab) and administration of an additional therapeutic agent (e.g., tocilizumab) occur within about two days of each other. In one embodiment, administration of the anti-OSMRβ antibody (e.g., vixarelimab) and administration of an additional therapeutic agent (e.g., tocilizumab) occur within about three days of each other. In one embodiment, administration of the anti-OSMRβ antibody (e.g., vixarelimab) and administration of an additional therapeutic agent (e.g., tocilizumab) occur within about four days of each other. In one embodiment, administration of the anti-OSMRβ antibody (e.g., vixarelimab) and administration of an additional therapeutic agent (e.g., tocilizumab) occur within about five days of each other. In one embodiment, administration of the anti-OSMRβ antibody (e.g., vixarelimab) and administration of an additional therapeutic agent (e.g., tocilizumab) occur within about six days of each other.

In some embodiments, the anti-OSMRβ antibody (e.g., vixarelimab) is administered to the subject after the subject has been treated with the second therapeutic agent (e.g., tocilizumab) for at least 1 week. In some embodiments, the anti-OSMRβ antibody (e.g., vixarelimab) is administered to the subject after the subject has been treated with the second therapeutic agent (e.g., tocilizumab) for at least 1 month In some embodiments, the anti-OSMRβ antibody (e.g., vixarelimab) is administered to the subject after the subject has been treated with the second therapeutic agent (e.g., tocilizumab) for at least 6 months. In some embodiments, the anti-OSMRβ antibody (e.g., vixarelimab) is administered to the subject after the subject has been treated with the second therapeutic agent (e.g., tocilizumab) for at least 1 year. In some embodiments, the anti-OSMRβ antibody (e.g., vixarelimab) is administered to the subject after the subject has been treated with the second therapeutic agent (e.g., tocilizumab) for at least 3 years. In some embodiments, the anti-OSMRβ antibody (e.g., vixarelimab) is administered to the subject after the subject has been treated with the second therapeutic agent (e.g., tocilizumab) for at least 5 years.

### IL-6

Interleukin 6 (IL-6) is an interleukin that acts as both a pro-inflammatory cytokine and an anti-inflammatory myokine. In some embodiments, the anti-OSMRβ antibody is administered before, during, or after administration with the second therapeutic agent. In some embodiments, the anti-OSMRβ antibody is administered before administration with the second therapeutic agent. In some embodiments, the anti-OSMRβ antibody is administered during administration with the second therapeutic agent. In some embodiments, the anti-OSMRβ antibody is administered after administration with the second therapeutic agent. In some embodiments, the second therapeutic agent is an anti-IL-6 antibody or an anti-IL-6 receptor antagonist. In some embodiments, the second therapeutic agent is an anti-IL-6 antibody or an anti-IL-6 receptor antibody.

In some embodiments, the heavy chain of the anti-IL-6 antibody or the anti-IL-6 receptor antagonist comprises the amino acid sequence of SEQ ID NO: 13. In some embodiments, the light chain of the anti-IL-6 antibody or the anti-IL-6 receptor antagonist comprises the amino acid sequence of SEQ ID NO: 14. In some embodiments, the heavy chain of the anti-IL-6 receptor antibody comprises the amino acid sequence of SEQ ID NO: 13. In some embodiments, the light chain of the anti-IL-6 receptor antibody comprises the amino acid sequence of SEQ ID NO: 14.In some embodiments, the heavy chain of the anti-IL-6 receptor antibody comprises the amino acid sequence of SEQ ID NO: 13, and the light chain of the anti-IL-6 receptor antibody comprises the amino acid sequence of SEQ ID NO: 14. In other embodiments, the anti-IL-6 antibody or the anti-IL-6 receptor antibody is tocilizumab. In some embodiments, the anti-IL-6 antibody or the anti-IL-6 receptor antagonist comprises the six CDRs of tocilizumab. In other embodiments, the anti-IL-6 receptor antibody is tocilizumab. In some embodiments, the anti-IL-6 receptor antibody comprises the six CDRs of tocilizumab.

In some embodiments of any of the above methods, the method further comprises administering a therapeutically effective amount of tocilizumab. Tocilizumab (Actemra®/RoActemra®) is a recombinant, humanized, anti-human monoclonal antibody directed against soluble and membrane-bound IL 6R, which inhibits IL-6 mediated signaling.

TABLE 2

| | SEQ ID | |
|---|---|---|
| | NO | Sequence |
| Tocilizumab | 13 | EVQLQESGPGLVRPSQTLSL |
| Heavy chain | | TCTVSGYSITSDHAWSWVRQ |
| | | PPGRGLEWIGYISYSGITTY |
| | | NPSLKSRVTMLRDTSKNQFS |
| | | LRLSSVTAADTAVYYCARSL |
| | | ARTTAMDYWGQGSLVTVSSA |
| | | STKGPSVFPLAPSSKSTSGG |
| | | TAALGCLVKDYFPEPVTVSW |
| | | NSGALTSGVHTFPAVLQSSG |
| | | LYSLSSVVTVPSSSLGTQTY |
| | | ICNVNHKPSNTKVDKKVEPK |
| | | SCDKTHTCPPCPAPELLGGP |
| | | SVFLEPPKPKDTLMISRTPE |
| | | VTCVVVDVSHEDPEVKFNWY |
| | | VDGVEVHNAKTKPREEQYNS |
| | | TYRVVSVLTVLHQDWLNGKE |
| | | YKCKVSNKALPAPIEKTISK |
| | | AKGQPREPQVYTLPPSRDEL |
| | | TKNQVSLTCLVKGFYPSDIA |
| | | VEWESNGQPENNYKTTPPVL |
| | | DSDGSFFLYSKLTVDKSRWQ |
| | | QGNVESCSVMHEALHNHYTQ |
| | | KSLSLSPG |
| Tocilizumab | 14 | DIQMTQSPSSLSASVGDRVT |
| Light chain | | ITCRASQDISSYLNWYQQKP |
| | | GKAPKLLIYYTSRLHSGVPS |
| | | RFSGSGSGTDFTFTISSLQP |
| | | EDIATYYCQQGNTLPYTFGQ |
| | | GTKVEIKRTVAAPSVFIFPP |
| | | SDEQLKSGTASVVCLLNNFY |
| | | PREAKVQWKVDNALQSGNSQ |
| | | ESVTEQDSKDSTYSLSSTLT |
| | | LSKADYEKHKVYACEVTHQG |
| | | LSSPVTKSFNRGEC |

In one aspect, the present disclosure provides a method of treating a pulmonary fibrotic disease in a subject in need thereof, the method comprising administering to the subject a therapeutically effective amount of (a) an anti-OSMRβ antibody and (b) an anti-IL-6 receptor antibody. In another aspect, the present disclosure provides use of a therapeutically effective amount of (a) an anti-OSMRβ antibody and (b) an anti-IL-6 receptor antibody in the manufacture of a medicament for the treatment of a pulmonary fibrotic disease in a subject in need thereof. In a further aspect, the present disclosure provides a therapeutically effective amount of (a) an anti-OSMRβ antibody and (b) an anti-IL-6 receptor antibody for use in treating a pulmonary fibrotic disease in a subject in need thereof. In some embodiments, the pulmonary fibrotic disease is selected from the group consisting of progressive pulmonary fibrosis (PPF), idiopathic pulmonary fibrosis (IPF), and systemic sclerosis-interstitial lung disease (SSc-ILD). In some embodiments, the pulmonary fibrotic disease is progressive pulmonary fibrosis (PPF). In some embodiments, the pulmonary fibrotic disease is idiopathic pulmonary fibrosis (IPF). In some embodiments, the pulmonary fibrotic disease is systemic sclerosis-interstitial lung disease (SSc-ILD).

In one aspect, the present disclosure provides a method of treating a lung inflammatory and/or fibrotic disease in a subject in need thereof, the method comprising administering to the subject a therapeutically effective amount of (a) an anti-OSMRβ antibody and (b) an anti-IL-6 receptor antibody. In another aspect, the present disclosure provides use of a therapeutically effective amount of (a) an anti-OSMRβ antibody and (b) an anti-IL-6 receptor antibody in the manufacture of a medicament for the treatment of a lung inflammatory and/or fibrotic disease in a subject in need thereof. In a further aspect, the present disclosure provides a therapeutically effective amount of (a) an anti-OSMRβ antibody and (b) an anti-IL-6 receptor antibody for use in treating a lung inflammatory and/or fibrotic disease in a subject in need thereof.

In one aspect, the present disclosure provides a method of treating a pulmonary fibrotic disease in a subject in need thereof, the method comprising administering to the subject a therapeutically effective amount of vixarelimab and tocilizumab. In another aspect, the present disclosure provides use of a therapeutically effective amount of vixarelimab and tocilizumab in the manufacture of a medicament for the treatment of a pulmonary fibrotic disease in a subject in need thereof. In a further aspect, the present disclosure provides a therapeutically effective amount of vixarelimab and tocilizumab for use in treating a pulmonary fibrotic disease in a subject in need thereof. In some embodiments, the pulmonary fibrotic disease is selected from the group consisting of progressive pulmonary fibrosis (PPF), idiopathic pulmonary fibrosis (IPF), and systemic sclerosis-interstitial lung disease (SSc-ILD). In some embodiments, the pulmonary fibrotic disease is progressive pulmonary fibrosis (PPF). In some embodiments, the pulmonary fibrotic disease is idiopathic pulmonary fibrosis (IPF). In some embodiments, the pulmonary fibrotic disease is systemic sclerosis-interstitial lung disease (SSc-ILD). Vixarelimab can be administered at any of the doses disclosed herein, any of the dosing frequencies disclosed herein, or any combination of dose and frequency disclosed herein.

In one aspect, the present disclosure provides a method of treating a lung inflammatory and/or fibrotic disease in a subject in need thereof, the method comprising administering to the subject a therapeutically effective amount of vixarelimab and tocilizumab. In another aspect, the present disclosure provides use of a therapeutically effective amount of vixarelimab and tocilizumab in the manufacture of a medicament for the treatment of a lung inflammatory and/or fibrotic disease in a subject in need thereof. In a further aspect, the present disclosure provides a therapeutically effective amount of vixarelimab and tocilizumab for use in treating a lung inflammatory and/or fibrotic disease in a subject in need thereof. Vixarelimab can be administered at any of the doses disclosed herein, any of the dosing frequencies disclosed herein, or any combination of dose and frequency disclosed herein. In some embodiments of any of the above aspects, the subject is human.

### Administration and Formulation

An anti-OSMRβ antibody of the disclosure (and any additional therapeutic agent) can be administered by any suitable means, including subcutaneous or intravenous injections, or parenteral or intrapulmonary, and, if desired for local treatment, intralesional administration. Parenteral infusions include intramuscular, intravenous, intraarterial, intraperitoneal, or, preferably for anti-OSMRβ, subcutaneous administration. Dosing can be by any suitable route, e.g., by injections, such as intravenous or subcutaneous injections, depending in part on whether the administration is brief or chronic. In preferred embodiments, the administration of an anti-OSMRβ antibody described herein is subcutaneous. Various dosing schedules including but not limited to single or multiple administrations over various time-points, bolus administration, and pulse infusion are contemplated herein.

Anti-OSMRβ antibodies of the disclosure would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the use of administration, the scheduling of administration, and other factors known to medical practitioners. The antibody may optionally be formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the disclosure (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician.

The antibody is suitably administered to the patient at one time or over a series of treatments. Depending on the type and severity of the disease. One exemplary dosage of the anti-OSMRβ antibody would be in the range from about 360 to 720 mg. Thus, one or more doses of 360 mg, 540, or 720 mg may be administered to the patient. In some embodiments, the anti-OSMRβ antibody is administered at a dose of 360 mg. In some embodiments, the anti-OSMRβ antibody is administered at a dose of 540 mg. In some embodiments, the anti-OSMRβ antibody is administered at a dose of 720 mg. Such doses may be administered intermittently, e.g., every week, every 2 weeks, every 3 weeks or every 4 weeks. In some embodiments, the anti-OSMRβ antibody is administered once a week. In some embodiments, the anti-OSMRβ antibody is administered once every 2 weeks. In some embodiments, the anti-OSMRβ antibody is administered once every 3 weeks. In some embodiments, the anti-OSMRβ antibody is administered once every 4 weeks. In some embodiments, the anti-OSMRβ antibody is administered once a month. An initial higher loading dose, followed by one or more lower doses may be administered. However, other dosage regimens may be useful. In some embodiments, the anti-OSMRβ is not administered at an initial higher loading dose. The progress of this therapy is easily monitored by conventional techniques and assays.

In preferred embodiments, the method comprises administering the anti-OSMRβ antibody at a dose of 360 mg to the patient every 2 weeks. In some embodiments, the method comprises administering to the subject 360 mg of the anti-OSMRβ antibody once every 1 week. In some embodiments, the method comprises administering to the subject 360 mg of the anti-OSMRβ antibody once every 3 weeks. In some embodiments, the method comprises administering to the subject 360 mg of the anti-OSMRβ antibody once every 4 weeks. In some embodiments, the method comprises administering to the subject 360 mg of the anti-OSMRβ antibody once every month. In some embodiments, the method comprises administering to the subject 540 mg of the anti-OSMRβ antibody once every 1 week. In some embodiments, the method comprises administering to the subject 540 mg once every 2 weeks. In some embodiments, the method comprises administering to the subject 540 mg of the anti-OSMRβ antibody once every 3 weeks. In some embodiments, the method comprises administering to the subject 540 mg of the anti-OSMRβ antibody once every 4 weeks. In some embodiments, the method comprises administering to the subject 540 mg of the anti-OSMRβ antibody once every month. In some embodiments, the method comprises administering to the subject 720 mg of the anti-OSMRβ antibody once every 1 week. In some embodiments, the method comprises administering to the subject 720 mg of the anti-OSMRβ antibody once every 2 weeks. In some embodiments, the method comprises administering to the subject 720 mg of the anti-OSMRβ antibody once every 3 weeks. In some embodiments, the method comprises administering to the subject 720 mg of the anti-OSMRβ antibody once every 4 weeks. In some embodiments, the method comprises administering to the subject 720 mg of the anti-OSMRβ antibody once every month.

In some embodiments, the method comprises treating the subject wherein the subject prior to treatment with the anti-OSMRβ antibody has a percentage of predicted forced vital capacity (% FVC) of about 35% to 90%. In some embodiments, the method comprises treating the subject wherein the subject prior to treatment with the anti-OSMRβ antibody has a percentage of predicted FVC of about 35% to 75%. In some embodiments, the method comprises treating the subject wherein the subject prior to treatment with the anti-OSMRβ antibody has a percentage of predicted FVC of about 35% to 50%. In some embodiments, the method comprises treating the subject wherein the subject prior to treatment with the anti-OSMRβ antibody has a percentage of predicted FVC of about 45% to 55%. In some embodiments, the method comprises treating the subject wherein the subject prior to treatment with the anti-OSMRβ antibody has a percentage of predicted FVC of about 30% to 60%. In some embodiments, the method comprises treating the subject wherein the subject prior to treatment with the anti-OSMRβ antibody has a percentage of predicted FVC of about 50% to 90%. In some embodiments, the method comprises treating the subject wherein the subject prior to treatment with the anti-OSMRβ antibody has a percentage of predicted FVC of about 50% to 75%. In some embodiments, the method comprises treating the subject wherein the subject prior to treatment with the anti-OSMRβ antibody has a percentage of predicted FVC of about 40% to 45%. In some embodiments, the method comprises treating the subject wherein the subject prior to treatment with the anti-OSMRβ antibody has a percentage of predicted FVC of about 40% to 50%. In some embodiments, the method comprises treating the subject wherein the subject prior to treatment with the anti-OSMRβ antibody has a percentage of predicted FVC of about 45% to 50%. In some embodiments, the method comprises treating the subject wherein the subject prior to treatment with the anti-OSMRβ antibody has a percentage of predicted FVC of about 45% to about 50%. In other embodiments, the subject has a FVC of about 45% predicted. In some embodiments, the % FVC is measured using spirometry.

In some embodiments, the subject has a forced expiratory volume in 1 second (FEV1)-to-forced vital capacity (FVC) ratio of about 0.35 to 0.70, prior to treatment with the anti-OSMRβ antibody. In some embodiments, the subject has a FEV1-to-FVC ratio of about 0.50 to 0.70, prior to treatment with the anti-OSMRβ antibody. In some embodiments, the subject has a FEV1-to-FVC ratio of about 0.60 to 0.70, prior to treatment with the anti-OSMRβ antibody. In some embodiments, the subject has a FEV1-to-FVC ratio of about 0.35 to 0.50, prior to treatment with the anti-OSMRβ antibody. In some embodiments, the subject has a FEV1-to-FVC ratio of about 0.40 to 0.50, prior to treatment with the anti-OSMRβ antibody. In some embodiments, the subject has a FEV1-to-FVC ratio of about 0.50 to 0.60, prior to treatment with the anti-OSMRβ antibody. In some embodiments, the subject has a FEV1-to-FVC ratio of about 0.60 to 0.70, prior to treatment with the anti-OSMRβ antibody. In some embodiments, the subject has a FEV1-to-FVC ratio of about 0.70 to 0.80, prior to treatment with the anti-OSMRβ antibody. In other embodiments, the subject has a FEV1-FVC ratio of about 0.70 to 0.80, prior to treatment with the anti-OSMRβ antibody. In preferred embodiments, the subject has FVC of about 45% predicted or greater. In preferred embodiments, the subject a FEV1-to-FVC ratio greater than about 0.70 prior to treatment with the anti-OSMRβ antibody.

In some embodiments, after administration of the anti-OSMRβ antibody the change in FVC is a decrease in FVC of less than 25 mL. In some embodiments, after administration of the anti-OSMRβ antibody the change in FVC is a decrease in FVC of less than 50 mL. In some embodiments, after administration of the anti-OSMRβ antibody the change in FVC is a decrease in FVC of less than 75 mL. In some embodiments, after administration of the anti-OSMRβ antibody the change in FVC is a decrease in FVC of less than 100 mL. In some embodiments, after administration of the anti-OSMRβ antibody the change in FVC is a decrease in FVC of less than 125 mL. In some embodiments, after administration of the anti-OSMRβ antibody the change in FVC is a decrease in FVC of less than 150 mL. In some embodiments, after administration of the anti-OSMRβ antibody the change in FVC is a decrease in FVC of less than 175 mL. In some embodiments, after administration of the anti-OSMRβ antibody the change in FVC is a decrease in FVC of less than 200 mL. In some embodiments, after administration of the anti-OSMRβ antibody the change in FVC is a decrease in FVC of less than 225 mL. In some embodiments, after administration of the anti-OSMRβ antibody the change in FVC is a decrease in FVC of less than 250 mL.

In some embodiments, after administration of the anti-OSMRβ antibody the change in FVC is an increase in FVC of at least 25 mL. In some embodiments, after administration of the anti-OSMRβ antibody the change in FVC is an increase in FVC of at least 50 mL. In some embodiments, after administration of the anti-OSMRβ antibody the change in FVC is an increase in FVC of at least 75 mL. In some embodiments, after administration of the anti-OSMRβ antibody the change in FVC is an increase in FVC of at least 100 mL. In some embodiments, after administration of the anti-OSMRβ antibody the change in FVC is an increase in FVC of at least 125 mL. In some embodiments, after administration of the anti-OSMRβ antibody the change in FVC is an increase in FVC of at least 150 mL. In some embodiments, after administration of the anti-OSMRβ antibody the change in FVC is an increase in FVC of at least 175 mL. In some embodiments, after administration of the anti-OSMRβ antibody the change in FVC is an increase in FVC of at least 200 mL. In some embodiments, after administration of the anti-OSMRβ antibody the change in FVC is an increase in FVC of at least 225 mL. In some embodiments, after administration of the anti-OSMRβ antibody the change in FVC is an increase in FVC of at least 250 mL. In some embodiments, after administration of the anti-OSMRβ antibody the change in FVC is an increase in FVC of at most 500 mL.

In some embodiments, the method is sufficient to produce an increase in the DL_{CO} percent (DL_{CO} %) predicted compared to the baseline measurement. In some embodiments, the increase in DL_{CO} or DL_{CO} % predicted is at least 5% above the respective baseline measurement over a treatment period. In some embodiments, the increase in DL_{CO} or DL_{CO} % predicted is at least 10% above the respective baseline measurement over a treatment period. In some embodiments, the increase in DL_{CO} or DL_{CO} % predicted is at least 15% above the respective baseline measurement over a treatment period. In some embodiments, the increase in DL_{CO} or DL_{CO} % predicted is at least 20% above the respective baseline measurement over a treatment period. In some embodiments, the increase in DL_{CO} or DL_{CO} % predicted is at least 25% above the respective baseline measurement over a treatment period. In some embodiments, the increase in DL_{CO} or DL_{CO} % predicted is at least 30% above the respective baseline measurement over a treatment period. In some embodiments, the increase in DL_{CO} or DL_{CO} % predicted is at least 35% above the respective baseline measurement over a treatment period. In some embodiments, the increase in DL_{CO} or DL_{CO} % predicted is at least 40% above the respective baseline measurement over a treatment period. In some embodiments, the increase in DL_{CO} or DL_{CO} % predicted is at least 45% above the respective baseline measurement over a treatment period. In some embodiments, the increase in DL_{CO} or DL_{CO} % predicted is at least 50% above the respective baseline measurement over a treatment period. In some embodiments, the increase in DL_{CO} or DL_{CO} % predicted is at least 55% above the respective baseline measurement over a treatment period. In some embodiments, the increase in DL_{CO} or DL_{CO} % predicted is at least 60% above the respective baseline measurement over a treatment period. In some embodiments, the increase in DL_{CO} or DL_{CO} % predicted is at least 65%. In some embodiments, the increase in DL_{CO} or DL_{CO} % predicted is at least 70% above the respective baseline measurement over a treatment period. In some embodiments, the increase in DL_{CO} or DL_{CO} % predicted is at least 75% above the respective baseline measurement over a treatment period. In some embodiments, the increase in DL_{CO} or DL_{CO} % predicted is at least 80% above the respective baseline measurement over a treatment period. In some embodiments, the increase in DL_{CO} or DL_{CO} % predicted is at least 90% above the respective baseline measurement over a treatment period. In some embodiments, the increase in DL_{CO} or DL_{CO} % predicted is at least 100% above the respective baseline measurement over a treatment period. In some embodiments, the increase in DL_{CO} or DL_{CO} % predicted is at least 110% above the respective baseline measurement over a treatment period. In some embodiments, the increase in DL_{CO} or DL_{CO} % predicted is at least 120% above the respective baseline measurement over a treatment period. In some embodiments, the increase in DL_{CO} or DL_{CO} % predicted is no more than 100% above the respective baseline measurement over a treatment period. In some embodiments, the increase in DL_{CO} or DL_{CO} % predicted is no more than 110% above the respective baseline measurement over a treatment period. In some embodiments, the increase in DL_{CO} or DL_{CO} % predicted is no more than 120% above the respective baseline measurement over a treatment period. In some embodiments, the increase in DL_{CO} or DL_{CO} % predicted is no more than 130% above the respective baseline measurement over a treatment period. In some embodiments, the increase in DL_{CO} or DL_{CO} % predicted is no more than 140% above the respective baseline measurement over a treatment period.

In some embodiments, the method is sufficient to reduce a decrease in the DL_{CO} percent (DL_{CO} %) predicted compared to the baseline measurement. In some embodiments, the decrease in DL_{CO} or DL_{CO} % predicted is less than 5% below the respective baseline measurement over a treatment period. In some embodiments, the decrease in DL_{CO} or DL_{CO} % predicted is less than 7% below the respective baseline measurement over a treatment period. In some embodiments, the decrease in DL_{CO} or DL_{CO} % predicted is less than 10% below the respective baseline measurement over a treatment period. In some embodiments, the decrease in DL_{CO} or DL_{CO} % predicted is less than 15% below the respective baseline measurement over a treatment period. In some embodiments, the decrease in DL_{CO} or DL_{CO} % predicted is less than 20% below the respective baseline measurement over a treatment period.

In some embodiments, the treatment period is about 6 weeks. In some embodiments, the treatment period is about 12 weeks. In some embodiments, the treatment period is about 24 weeks. In some embodiments, the treatment period is about 36 weeks. In some embodiments, the treatment period is about 48 weeks. In some embodiments, the treatment period is about 60 weeks. In some embodiments, the treatment period is about 72 weeks. In other embodiments, the treatment period is about 52 weeks.

In some embodiments, the method comprises treating the subject having a pulmonary fibrotic disorder, wherein administering the dose of anti-OSMRβ antibody to the subject results in a change in distance traveled by the subject in the 6-minute walk test (6MWT), wherein the change is the difference in the distance traveled by the subject in the 6MWT performed at 2 time points in a treatment period wherein the first time point is at the first administration of the anti-OSMRβ antibody and the second time point is the time at which a later dose of the anti-OSMRβ antibody is administered. In some embodiments, the difference in the distance traveled by the subject in the 6MWT performed at the 2 time points is a decrease of less than about 5%. In some embodiments, the difference in the distance traveled by the subject in the 6MWT performed at the 2 time points is a decrease of less than about 10%, In some embodiments, the difference in the distance traveled by the subject in the 6MWT performed at the 2 time points is a decrease of less than about 15%. In some embodiments, the difference in the distance traveled by the subject in the 6MWT performed at the 2 time points is a decrease of less than about 20%. In some embodiments, the difference in the distance traveled by the subject in the 6MWT performed at the 2 time points is a decrease of less than about 25%. In some embodiments, the difference in the distance traveled by the subject in the 6MWT performed at the 2 time points is a decrease of less than about 30%. In some embodiments, the difference in the distance traveled by the subject in the 6MWT performed at the 2 time points is a decrease of less than about 40%. In some embodiments, the difference in the distance traveled by the subject in the 6MWT performed at the 2 time points is a decrease of less than about 50%. In some embodiments, the difference in the distance traveled by the subject in the 6MWT performed at the 2 time points is a decrease of less than about 25 m. In some embodiments, the difference in the distance traveled by the subject in the 6MWT performed at the 2 time points is a decrease of less than about 35 m. In some embodiments, the difference in the distance traveled by the subject in the 6MWT performed at the 2 time points is a decrease of less than about 50 m. In some embodiments, the difference in the distance traveled by the subject in the 6MWT performed at the 2 time points is an increase of at least about 5%. In some embodiments, the difference in the distance traveled by the subject in the 6MWT performed at the 2 time points is an increase of at least about 10%. In some embodiments, the difference in the distance traveled by the subject in the 6MWT performed at the 2 time points is an increase of at least about 15%. In some embodiments, the difference in the distance traveled by the subject in the 6MWT performed at the 2 time points is an increase of at least about 20%. In some embodiments, the difference in the distance traveled by the subject in the 6MWT performed at the 2 time points is an increase of at least about 25%. In some embodiments, the difference in the distance traveled by the subject in the 6MWT performed at the 2 time points is an increase of at least about 30%. In some embodiments, the difference in the distance traveled by the subject in the 6MWT performed at the 2 time points is an increase of at least about 40%. In some embodiments, the difference in the distance traveled by the subject in the 6MWT performed at the 2 time points is an increase of at least about 50%. In some embodiments, the difference in the distance traveled by the subject in the 6MWT performed at the 2 time points is an increase of about 5% to about 50%. In some embodiments, the difference in the distance traveled by the subject in the 6MWT performed at the 2 time points is an increase of about 5% to about 40%. In some embodiments, the difference in the distance traveled by the subject in the 6MWT performed at the 2 time points is an increase of about 5% to about 30%. In some embodiments, the difference in the distance traveled by the subject in the 6MWT performed at the 2 time points is an increase of about 5% to about 20%. In some embodiments, the difference in the distance traveled by the subject in the 6MWT performed at the 2 time points is an increase of about 5% to about 10%. In some embodiments, the difference in the distance traveled by the subject in the 6MWT performed at the 2 time points is an increase of about 10% to about 50%. In some embodiments, the difference in the distance traveled by the subject in the 6MWT performed at the 2 time points is an increase of about 10% to about 40%. In some embodiments, the difference in the distance traveled by the subject in the 6MWT performed at the 2 time points is an increase of about 10% to about 30%. In some embodiments, the difference in the distance traveled by the subject in the 6MWT performed at the 2 time points is an increase of about 10% to about 20%. In some embodiments, the difference in the distance traveled by the subject in the 6MWT performed at the 2 time points is an increase of about 20% to about 50%. In some embodiments, the difference in the distance traveled by the subject in the 6MWT performed at the 2 time points is an increase of about 20% to about 40%. In some embodiments, the difference in the distance traveled by the subject in the 6MWT performed at the 2 time points is an increase of about 20% to about 30%. In some embodiments, the difference in the distance traveled by the subject in the 6MWT performed at the 2 time points is an increase of about 30% to about 50%. In some embodiments, the difference in the distance traveled by the subject in the 6MWT performed at the 2 time points is an increase of about 30% to about 40%. In some embodiments, the difference in the distance traveled by the subject in the 6MWT performed at the 2 time points is an increase of about 40% to about 50%.

In some embodiments, the difference in the distance traveled by the subject in the 6MWT performed at the 2 time points is an increase of at least about 5 m. In some embodiments, the difference in the distance traveled by the subject in the 6MWT performed at the 2 time points is an increase of at least about 10 m. In some embodiments, the difference in the distance traveled by the subject in the 6MWT performed at the 2 time points is an increase of at least about 15 m. In some embodiments, the difference in the distance traveled by the subject in the 6MWT performed at the 2 time points is an increase of at least about 20 m. In some embodiments, the difference in the distance traveled by the subject in the 6MWT performed at the 2 time points is an increase of at least about 25 m. In some embodiments, the difference in the distance traveled by the subject in the 6MWT performed at the 2 time points is an increase of at least about 30 m. In some embodiments, the difference in the distance traveled by the subject in the 6MWT performed at the 2 time points is an increase of at least about 35 m. In some embodiments, the difference in the distance traveled by the subject in the 6MWT performed at the 2 time points is an increase of at least about 40 m. In some embodiments, the difference in the distance traveled by the subject in the 6MWT performed at the 2 time points is an increase of at least about 45 m. In some embodiments, the difference in the distance traveled by the subject in the 6MWT performed at the 2 time points is an increase of no more than about 50 m. In some embodiments, the difference in the distance traveled by the subject in the 6MWT performed at the 2 time points is an increase of about 5 m to about 50 m. In some embodiments, the difference in the distance traveled by the subject in the 6MWT performed at the 2 time points is an increase of about 10 m to about 50 m. In some embodiments, the difference in the distance traveled by the subject in the 6MWT performed at the 2 time points is an increase of about 20 m to about 50 m. In some embodiments, the difference in the distance traveled by the subject in the 6MWT performed at the 2 time points is an increase of about 30 m to about 50 m. In some embodiments, the difference in the distance traveled by the subject in the 6MWT performed at the 2 time points is an increase of about 40 m to about 50 m. In some embodiments, the difference in the distance traveled by the subject in the 6MWT performed at the 2 time points is an increase of about 10 m to about 40 m. In some embodiments, the difference in the distance traveled by the subject in the 6MWT performed at the 2 time points is an increase of about 20 m to about 30 m.

In some aspects, a method of increasing the distance traveled, as measured the 6MWT, by a subject suffering from a pulmonary fibrotic disorder is provided, comprising administering the dose of an anti-OSMRβ antibody of the disclosure to the subject.

In some embodiments, the subject has been diagnosed with or determined to suffer from one or more pulmonary fibrotic disorders. In some embodiments, the pulmonary fibrotic disorder is idiopathic pulmonary fibrosis (IPF). In some embodiments, the pulmonary fibrotic disorder is progressive pulmonary fibrosis (PPF) (alternatively referred to as pulmonary fibrosis-interstitial lung disease (PF-ILD)). In some embodiments, the PPF is chronic fibrosing ILD (CF-ILD). In some embodiments, the PPF is CF-ILD with a progressive phenotype. In some embodiments, the PPF is interstitial lung disease (ILD). In some embodiments, the PPF is systemic sclerosis-ILD (SSC-ILD). In some embodiments, the PPF is drug-induced ILD. In some embodiments, the PPF is hypersensitivity pneumonitis. In some embodiments, the PPF is interstitial pneumonia with autoimmune features (IPAF). In some embodiments, the PPF is fibrosing interstitial pneumonia. In some embodiments, the PPF is unclassifiable ILD. In some embodiments, the pulmonary fibrotic disorder is chronic fibrosing interstitial lung diseases with a progressive phenotype.

In some embodiments, the pulmonary fibrosis is associated with one or more of the following: usual interstitial pneumonia, idiopathic interstitial pneumonia, desquamative interstitial pneumonia, respiratory bronchiolitis-interstitial lung disease, acute interstitial pneumonia, nonspecific interstitial pneumonia, sarcoidosis, cryptogenic organizing pneumonia, eosinophilic pneumonia, infection, exposure to occupational or environmental agents, cigarette smoking, interstitial lung disease induced by drugs or radiation, rheumatic disease-associated interstitial lung disease, lymphoid interstitial pneumonia, pleuropulmonary fibroelastosis, pulmonary Langerhans cell histiocytosis, systemic sclerosis-interstitial lung disease, Hermansky-Pudlak syndrome, and telomeropathy. In some embodiments, the pulmonary fibrosis is associated with usual interstitial pneumonia. In some embodiments, the pulmonary fibrosis is associated with idiopathic interstitial pneumonia. In some embodiments, the pulmonary fibrosis is associated with desquamative interstitial pneumonia. In some embodiments, the pulmonary fibrosis is associated with respiratory bronchiolitis-interstitial lung disease. In some embodiments, the pulmonary fibrosis is associated with acute interstitial pneumonia. In some embodiments, the pulmonary fibrosis is associated with nonspecific interstitial pneumonia. In some embodiments, the pulmonary fibrosis is associated with sarcoidosis. In some embodiments, the pulmonary fibrosis is associated with cryptogenic organizing pneumonia. In some embodiments, the pulmonary fibrosis is associated with eosinophilic pneumonia. In some embodiments, the pulmonary fibrosis is associated with infection. In some embodiments, the pulmonary fibrosis is associated with exposure to occupational. In some embodiments, the pulmonary fibrosis is associated with exposure to environmental agents. In some embodiments, the pulmonary fibrosis is associated with cigarette smoking. In some embodiments, the pulmonary fibrosis is associated with interstitial lung disease induced by drugs. In some embodiments, the pulmonary fibrosis is associated with interstitial lung disease induced by radiation. In some embodiments, the pulmonary fibrosis is associated with rheumatic disease-associated interstitial lung disease. In some embodiments, the pulmonary fibrosis is associated with lymphoid interstitial pneumonia. In some embodiments, the pulmonary fibrosis is associated with pleuropulmonary fibroelastosis. In some embodiments, the pulmonary fibrosis is associated with pulmonary Langerhans cell histiocytosis. In some embodiments, the pulmonary fibrosis is associated with systemic sclerosis-interstitial lung disease. In some embodiments, the pulmonary fibrosis is associated with Hermansky-Pudlak syndrome. In some embodiments, the pulmonary fibrosis is associated with telomeropathy.

In some embodiments, the subject has not been diagnosed with or is not experiencing an inflammatory bowel disease. In some embodiments, the subject has not been diagnosed with or is not experiencing a fibrotic skin disease. In some embodiments, the subject has not been diagnosed with or is not experiencing prurigo nodularis. In some embodiments, the subject has not been diagnosed with or is not experiencing AD.

### Exemplary Embodiments

Particular embodiments of the disclosure are set forth in the following numbered paragraphs:
1. A method for treating a pulmonary fibrotic disease comprising administering to a subject in need thereof a therapeutically effective dose of an anti-OSMRβ (oncostatin M receptor beta) antibody.
2. An anti-OSMRβ antibody for use in treating a pulmonary fibrotic disease in a subject in need thereof.
3. Use of an anti-OSMRβ antibody in the manufacture of a pharmaceutical composition for treating a pulmonary fibrotic disease in a subject in need thereof.
4. The method of embodiment 1, the anti-OSMRβ antibody for use of embodiment 2, or the use of embodiment 3, wherein the pulmonary fibrotic disease is selected from the group consisting of progressive pulmonary fibrosis (PPF), idiopathic pulmonary fibrosis (IPF), and systemic sclerosis-interstitial lung disease (SSc-ILD).
5. The method of embodiment 1 or 4, the anti-OSMRβ antibody for use of embodiment 2 or 4, or the use of embodiment 3 or 4, wherein the anti-OSMRβ antibody inhibits signaling of the type II OSMR by OSM and IL-31.
6. The method of any one of embodiments 1, 4 and 5, the anti-OSMRβ antibody for use of any one of embodiments 2, 4 and 5, or the use of any one of embodiments 3-5, wherein the anti-OSMRβ antibody is vixarelimab.
7. The method of any one of embodiments 1 and 4-6, the anti-OSMRβ antibody for use of any one of embodiments 2 and 4-6, or the use of any one of embodiments 3-6, wherein the therapeutically effective dose is about 360 mg to 720 mg of the anti-OSMRβ antibody.
8. The method of any one of embodiments 1 and 4-7, the anti-OSMRβ antibody for use of any one of embodiments 2 and 4-7, or the use of any one of embodiments 3-7, wherein the therapeutically effective dose is 360 mg of the anti-OSMRβ antibody.
9. The method of any one of embodiments 1 and 4-8, the anti-OSMRβ antibody for use of any one of embodiments 2 and 4-8, or the use of any one of embodiments 3-8, wherein the therapeutically effective dose of the anti-OSMRβ antibody is administered once per week, once every 2 weeks, once every 3 weeks, once every 4 weeks, or once every month.
10. The method of any one of embodiments 1 and 4-9, the anti-OSMRβ antibody for use of any one of embodiments 2 and 4-9, or the use of any one of embodiments 3-9, wherein the therapeutically effective dose of the anti-OSMRβ antibody is administered once every 2 weeks.
11. The method of any one of embodiments 1 and 4-10, the anti-OSMRβ antibody for use of any one of embodiments 2 and 4-10, or the use of any one of embodiments 3-10, wherein the therapeutically effective dose of the anti-OSMRβ antibody is administered subcutaneously or intravenously.
12. The method of any one of embodiments 1 and 4-11, the anti-OSMRβ antibody for use of any one of embodiments 2 and 4-11, or the use of any one of embodiments 3-11, wherein the therapeutically effective dose of the anti-OSMRβ antibody is administered subcutaneously.
13. The method of any one of embodiments 1 and 4-12, the anti-OSMRβ antibody for use of any one of embodiments 2 and 4-12, or the use of any one of embodiments 3-12, wherein prior to treatment with the anti-OSMRβ antibody the subject has a percentage of predicted forced vital capacity (% FVC) of about 35% to 90%, about 35% to 75%, about 35% to 50%, about 45% to 55%, about 30% to 60%, about 50% to 90%, about 50% to 75%, about 40% to 45%, about 40% to 50%, about 45% to 50%, or about 45% to about 50%.
14. The method of any one of embodiments 1 and 4-13, the anti-OSMRβ antibody for use of any one of embodiments 2 and 4-13, or the use of any one of embodiments 3-13, wherein prior to treatment with the anti-OSMRβ antibody the subject has a forced expiratory volume in 1 second (FEV1)-to-FVC ratio of about 0.35 to 0.70, about 0.50 to 0.70, about 0.60 to 0.70, about 0.35 to 0.50, about 0.40 to 0.50, about 0.50 to 0.60, about 0.60 to 0.70, about 0.70 to 0.80.
15. The method of any one of embodiments 1 and 4-14, the anti-OSMRβ antibody for use of any one of embodiments 2 and 4-14, or the use of any one of embodiments 3-14, wherein administering the dose of anti-OSMRβ antibody to the subject results in a change in FVC in the subject, wherein the change is a measure of absolute change in FVC in milliliters (ml) over a treatment period beginning the time at the first administration of the anti-OSMRβ antibody until the time at which a later dose of the anti-OSMRβ antibody is administered.
16. The method, anti-OSMRβ antibody for use or the use of embodiment 15, wherein the change in FVC during the treatment period is a decrease in FVC of less than 25 mL, 50 mL, 75 mL, 100 mL, 125 mL, 150 mL, 175 mL, or 200 mL, 225 mL or 250 mL or an increase in FVC of at least 25 mL, 50 mL, 75 mL, 100 mL, 125 mL, 150 mL, 175 mL, or 200 mL, 225 mL or 250 mL.
17. The method of any one of embodiments 1 and 4-16, the anti-OSMRβ antibody for use of any one of embodiments 2 and 4-16, or the use of any one of embodiments 3-16, wherein administering the dose of anti-OSMRβ antibody to the subject results in an increase in DLCO[Hb] in the subject, wherein the change is a measure of absolute change in DLCO[Hb] over a treatment period beginning at the time of the first administration of the anti-OSMRβ antibody until the time at which a later dose of the anti-OSMRβ antibody is administered.
18. The method, anti-OSMRβ antibody for use or use of embodiment 17, wherein the change in DLCO[Hb] during the treatment period is an increase of at least 5%, 10%, 15%, 20%, 25%, 30%, 35%, 40%, 45%, 50%, 55%, 60%, 65%, 70%, 75%, 80% or 90%.
19. The method of any one of embodiments 1 and 4-18, the anti-OSMRβ antibody for use of any one of embodiments 2 and 4-18, or the use of any one of embodiments 3-18, wherein administering the dose of anti-OSMRβ antibody to the subject results in a change in distance traveled by the subject in the 6-minute walk test (6MWT) wherein the change is a measure of the distance traveled by the subject in the 6MWT over a treatment period beginning at the time of the first administration of the anti-OSMRβ antibody until the time at which a later dose of the anti-OSMRβ antibody is administered.
20. The method, anti-OSMRβ antibody for use or use of embodiment 19, wherein the change in distance during the treatment period is an increase of at least 5%, 10%, 15%, 20%, 25%, or 30%.
21. The method, anti-OSMRβ antibody for use or use of embodiment 19, wherein the change in distance during the treatment period is a decrease of less than about 5%, 10%, 15%, 20%, 25%, or 30%
22. The method of any one of embodiments 1 and 4-21, the anti-OSMRβ antibody for use of any one of embodiments 2 and 4-21, or the use of any one of embodiments 3-21, wherein the administering the dose of anti-OSMRβ antibody to the subject results in a change in cough relative to baseline, wherein the change is a measure of the cough frequency over a treatment period beginning at the time of the first administration of the anti-OSMRβ antibody until the time at which a later dose of the anti-OSMRβ antibody is administered, wherein the change is a reduction in cough frequency, and wherein cough is measured by digital continuous ambulatory cough detection device.
23. The method, anti-OSMRβ antibody for use, or use of any one of embodiments 15 to 22, wherein the treatment period is about 6 weeks, about 12 weeks, about 24 weeks, about 36 weeks, about 48 weeks, about 60 weeks, or about 72 weeks.
24. The method of any one of embodiments 1 and 4-23, the anti-OSMRβ antibody for use of any one of embodiments 2 and 4-23, or the use of any one of embodiments 3-23, wherein the anti-OSMR antibody is administered to the subject in combination with a second therapeutic agent.
25. The method, anti-OSMRβ antibody for use, or use of embodiment 24, wherein the second therapeutic agent is a therapeutic agent indicated for a lung fibrotic disease or disorder.
26. The method, anti-OSMRβ antibody for use or use of embodiment 24 or 25, wherein the second therapeutic agent pirfenidone or nintedanib.
27. The method, anti-OSMRβ antibody for use, or use of embodiment 24 or 25, wherein the second therapeutic agent is an anti-IL-6 antibody or an anti-IL-6 receptor antibody.
28. The method, anti-OSMRβ antibody for use, or use of embodiment 27, wherein the anti-IL-6 receptor antibody is tocilizumab.
29. The method, anti-OSMRβ antibody for use, or use of embodiment 27, wherein the anti-IL-6 antibody or the anti-IL-6 receptor antibody comprises a heavy chain comprising the amino acid sequence of SEQ ID NO: 13.
30. The method, anti-OSMRβ antibody for use, or use of embodiment 27, wherein the anti-IL-6 antibody or the anti-IL-6 receptor antibody comprises a light chain comprising the amino acid sequence of SEQ ID NO: 14.
31. The method, anti-OSMRβ antibody for use, or use of embodiment 27, wherein the anti-IL-6 antibody or the anti-IL-6 receptor antibody comprises the six CDRs of tocilizumab.
32. The method of any one of embodiments 1 and 4-31, the anti-OSMRβ antibody for use of any one of embodiments 2 and 4-31, or the use of any one of embodiments 3-31, wherein the subject is human.
33. A method of treating a pulmonary fibrotic disease in a subject in need thereof, the method comprising administering to the subject a therapeutically effective amount of (a) an anti OSMRβ antibody and (b) an anti-IL-6 receptor antibody.
34. A therapeutically effective amount of (a) an anti OSMRβ antibody and (b) an anti-IL-6 receptor antibody for use in treating a pulmonary fibrotic disease in a subject in need thereof.
35. Use of a therapeutically effective amount of (a) an anti OSMRβ antibody and (b) an anti-IL-6 receptor antibody in the manufacture of a medicament for treating a pulmonary fibrotic disease in a subject in need thereof.
36. The method of embodiment 33, the anti-OSMRβ and anti-IL-6 receptor antibodies for use of embodiment 34, or the use of embodiment 35, wherein the anti-OSMRβ antibody comprises a heavy chain variable domain (VH) comprising SEQ ID NO:7 and a light chain variable domain (VL) comprising SEQ ID NO:8.
37. The method, anti-OSMRβ and anti-IL-6 receptor antibodies for use, or use of embodiment 36, wherein the anti-OSMRβ antibody comprises a heavy chain (HC) comprising SEQ ID NO:5 and a light chain (LC) comprising SEQ ID NO:6.
38. The method of embodiment 33, the anti-OSMRβ and anti-IL-6 receptor antibodies for use of embodiment 34, or the use of embodiment 35, wherein the anti-OSMRβ antibody is vixarelimab.
39. The method of any one of embodiments 33 and 36-38, the anti-OSMRβ and anti-IL-6 receptor antibodies for use of any one of embodiments 34 and 36-38, or the use of any one of embodiments 35-38, wherein the heavy chain of the anti-IL-6 receptor antibody comprises the amino acid sequence of SEQ ID NO: 13, and the light chain of the anti-IL-6 receptor antibody comprises the amino acid sequence of SEQ ID NO: 14.
40. The method of any one of embodiments 33 and 36-38, the anti-OSMRβ and anti-IL-6 receptor antibodies for use of any one of embodiments 34 and 36-38, or the use of any one of embodiments 35-38, wherein the anti-IL-6 receptor antibody is tocilizumab.
41. The method of any one of embodiments 33 and 36-38, the anti-OSMRβ and anti-IL-6 receptor antibodies for use of any one of embodiments 34 and 36-38, or the use of any one of embodiments 35-38, wherein the anti-IL-6 receptor antibody comprises the six CDRs of tocilizumab.
42. The method of embodiment 33, the anti-OSMRβ and anti-IL-6 receptor antibodies for use of embodiment 34, or the use of embodiment 35, wherein the anti-OSMRβ antibody is vixarelimab and the anti-IL-6 receptor antibody is tocilizumab.
43. The method of any one of embodiments 33 and 36-42, the anti-OSMRβ and anti-IL-6 receptor antibodies for use of any one of embodiments 34 and 36-42, or the use of any one of embodiments 35-42, wherein the pulmonary fibrotic disease is selected from the group consisting of progressive pulmonary fibrosis (PPF), idiopathic pulmonary fibrosis (IPF), and systemic sclerosis-interstitial lung disease (SSc-ILD).
44. The method, anti-OSMRβ and anti-IL-6 receptor antibodies for use, or use of embodiment 43, wherein the pulmonary fibrotic disease is IPF.
45. The method of any one of embodiments 33 and 36-44, the anti-OSMRβ and anti-IL-6 receptor antibodies for use of any one of embodiments 34 and 36-44, or the use of any one of embodiments 35-44, wherein the anti-OSMRβ antibody and the anti-IL-6 receptor antibody are administered simultaneously.
46. The method of any one of embodiments 33 and 36-44, the anti-OSMRβ and anti-IL-6 receptor antibodies for use of any one of embodiments 34 and 36-44, or the use of any one of embodiments 35-44, wherein the anti-OSMRβ antibody and the anti-IL-6 receptor antibody are administered sequentially.
47.The method any one of embodiments 33 and 36-45, the anti-OSMRβ and anti-IL-6 receptor antibodies for use of any one of embodiments 34 and 36-45, or the use of any one of embodiments 35-45, wherein the anti-OSMRβ antibody and the anti-IL-6 receptor antibody are administered in the same composition.
48. The method of any one of embodiments 33, 36-44 and 46, the anti-OSMRβ and anti-IL-6 receptor antibodies for use of any one of embodiments 34, 36-44 and 46, or the use of any one of embodiments 35-44 and 46, wherein the anti-OSMRβ antibody and the anti-IL-6 receptor antibody are administered in the different compositions.
49. The method of any one of embodiments 33, 36-44 and 46-48, the anti-OSMRβ and anti-IL-6 receptor antibodies for use of any one of embodiments 34, 36-44 and 46-48, or the use of any one of embodiments 35-44 and 46-48, wherein the subject is human.

### EXAMPLES

The disclosure will be further understood by reference to the following examples, which are intended to be purely exemplary of the disclosure herein. The present disclosure is not limited in scope by the exemplified embodiments, which are intended as illustrations of single aspects of the disclosure only.

### Example 1. Expression of OSM and OSMR in IPF

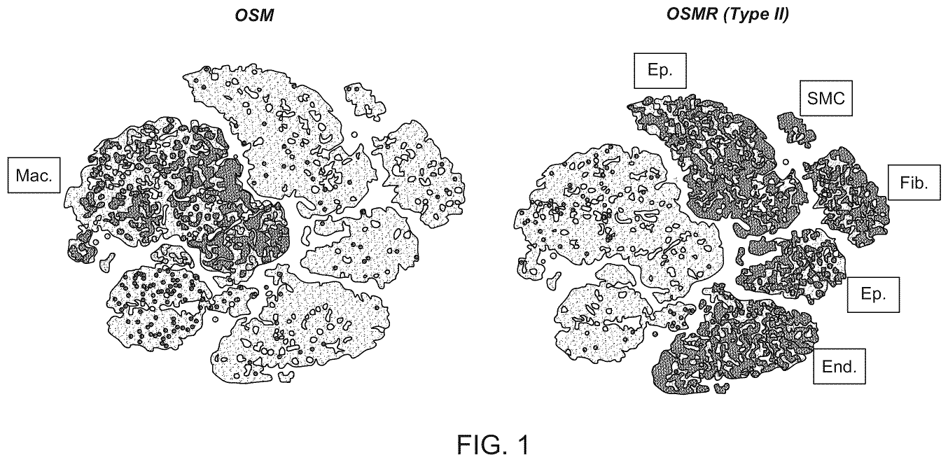

As shown in the FIG. 1, scRNA seq analysis of lung tissue isolated from a human subject diagnosed with IPF shows OSM expression in macrophages and OSMRβ expression in epithelial cells, single smooth muscle cell types (SMC), fibroblasts, and endothelial cells. Expression levels are positively correlated with increasing darkness of the shading. These data show that in lung tissue of subjects suffering from IPF, both OSM and OSMRβ are expressed, and it is reasonable to assume that OSMRβ activation upon binding of OSM occurs in IPF.

### Example 2. Blockade of OSM Reduces Lung Injury and Inflammation

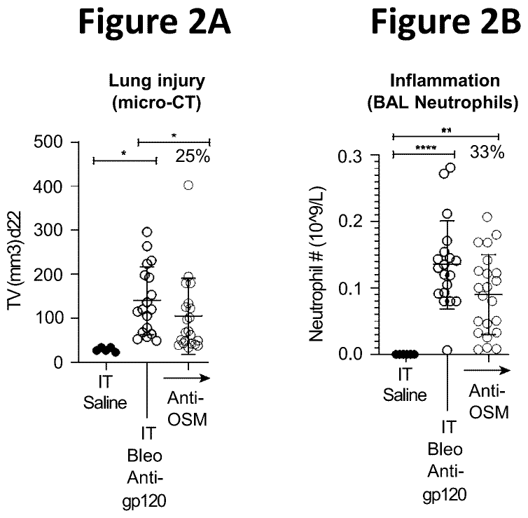

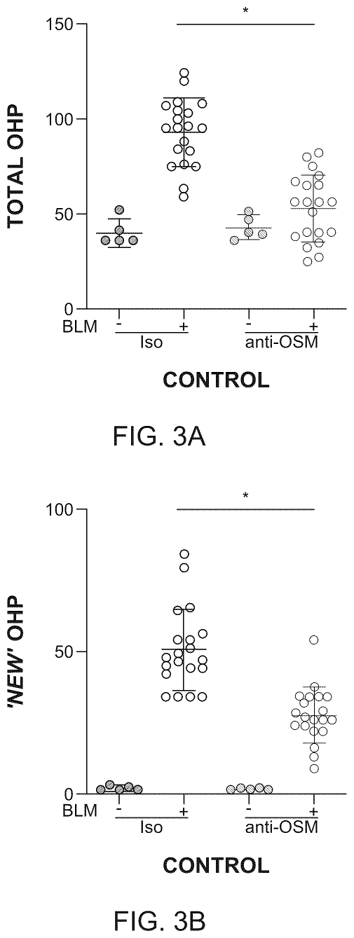

A mouse model of bleomycin (BLM)-induced pulmonary fibrosis (Sun et al., 2021, Sci Transl Med 13(605): eabe0407; Sun et al., 2019, JCI Insight. 2019;4(14):e128674) was used to perform experiments that demonstrate the effects of blocking OSM signaling by administration of anti-OSM antibody. Male C57BL/6J mice were dosed with 0.25 U/kg bleomycin intratracheally three times (Day 0, Day 2, Day 4). Mice were treated with a control or anti-OSM antibody twice per week starting on Day-4 with the last dose on Day 22 prior to the end of the study on Day 24 post intratracheal administration of bleomycin (Day 0). Specifically, the antibody treatments were injection of a control anti-gp120-mIgG2a antibody (800 ug) or of an anti-OSM-mIgG2a (mouse IgG2a, injected as a mix of 500 ug anti-OSM-mIgG2a and 300 anti-gp120-mIgG2a antibody) antibody. Disease was allowed to progress through Day 24 when the mice were sacrificed for disease endpoint assessment. Lung injury was measured by micro-CT imaging of the lung on Day 22. Increase Tissue Volume (mm³) reflects increased injury and disease in the lung of the bleomycin-treated mice (See FIG. 2A). Neutrophil infiltration into the lung following bleomycin-induced injury was measured in the bronchoalveolar lavage (BAL) fluid on Day 24 (see FIG. 2B). Increased tissue volume reflects increased injury and disease in the lung of the bleomycin-treated mice. This effect was reduced with anti-OSM treatment. Further, BLM-treated WT mice given anti-OSM blocking Ab's had significantly less total and new hydroxyproline deposited in the lung, compared to control Ab treated mice (See FIG. 3A and 3B).

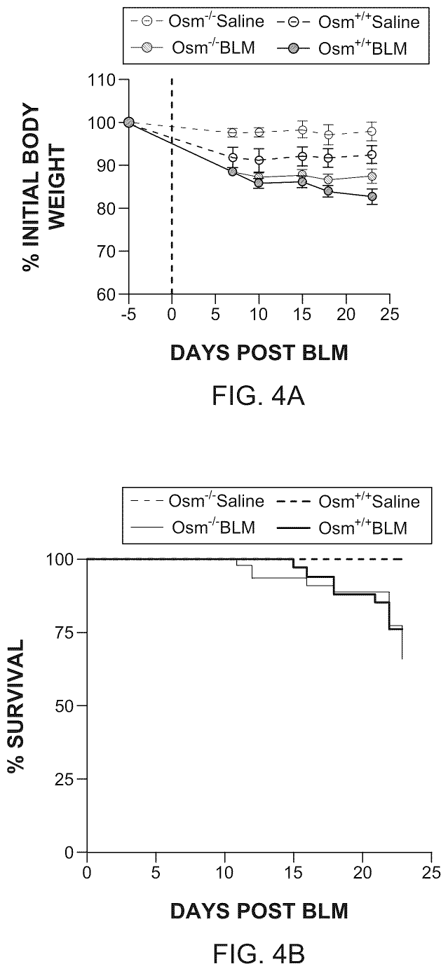

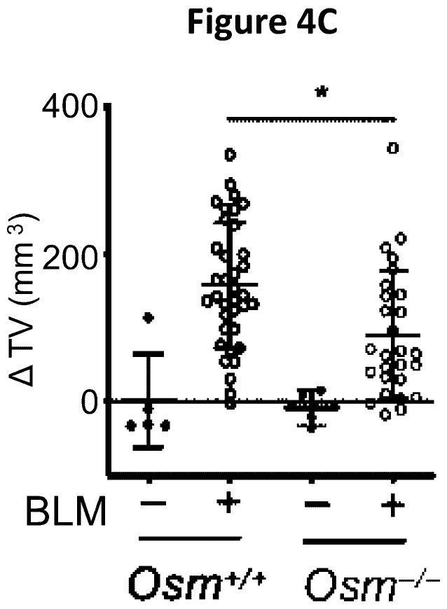

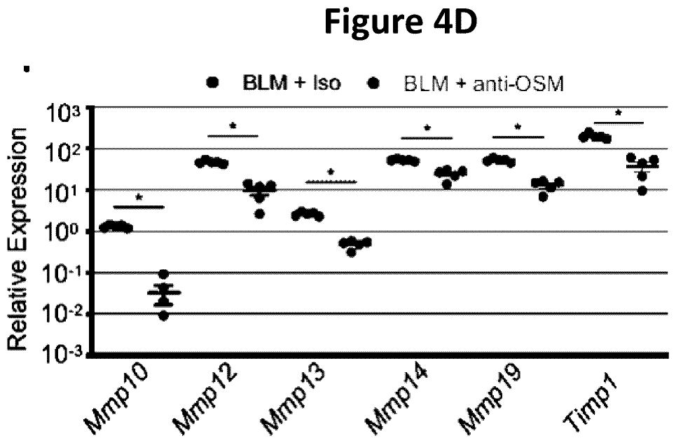

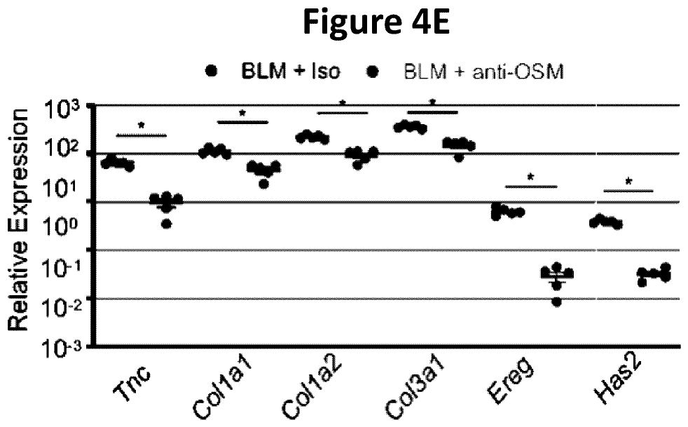

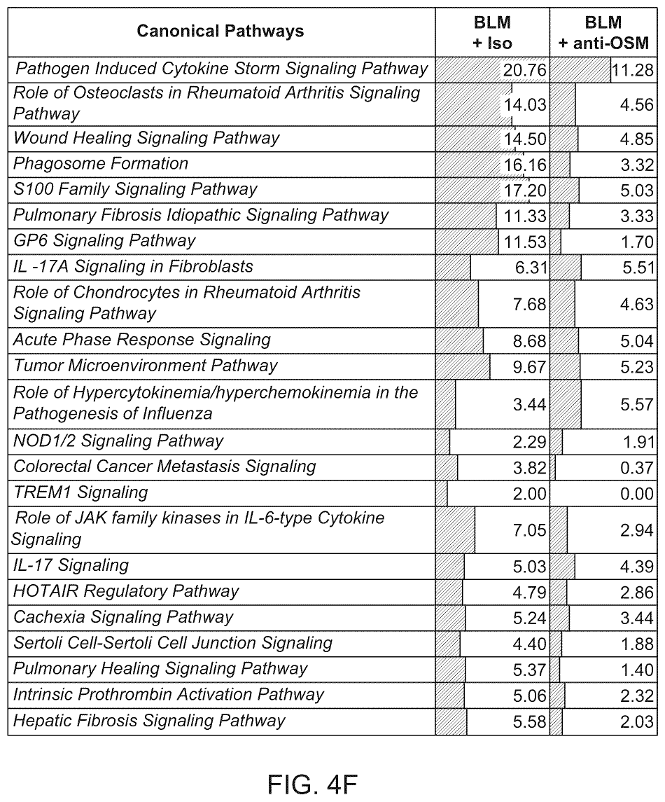

To formally test the requirement for OSM in BLM-induced pulmonary fibrosis Osm+/¬+ and Osm−/− mice received a solution of bleomycin (0.75 U/kg (DNC #0703-3155-01; EVA) prepared in PBS or saline was then instilled in the trachea. Bleomycin or saline control was administered equally in subtherapeutic doses over 3 separate days. Both Osm+/¬+ and Osm−/− mice had similar weight loss (FIG. 4A) and survival (FIG. 4B) post BLM, however Osm−/− mice had significantly less lung damage, determined by changes in tissue volume (TV) (FIG. 4C). In a separate group of mice, BLM-treated WT mice given anti-OSM blocking Ab or isotype control. Transcriptional analysis of lung tissue from anti-OSM treated mice identified a variety of fibrotic pathways that were reduced following OSM blockade, including significantly reduced extracellular matrix regulators (Timp1, Mmp10, -12, -13, -14 and -19)(Figure 4D), collagen synthesizing and regulating genes (Col1a1, Col1a2, Col3a1, Ereg, Has2) and most notably Tnc, which encodes the potent pro-fibrotic hexameric ECM glycoprotein, Tenascin-c48 (FIG. 4E). Finally, pathway analysis revealed the extent of the benefit of OSM blockade, with many wound-healing and fibrotic (“wound healing”, “IPF signaling”, “pulmonary healing”, “hepatic fibrosis”) pathways reduced (FIG. 4F).

### Example 3. Selecting a Dose and Schedule for IPF and SSc-ILD

The target efficacious concentration (C_{eff}) was estimated based on the combination of nonclinical data from cynomolgus monkey itch studies and clinical data from a phase 1 clinical study in subjects diagnosed with atopic dermatitis (AD) and adjusted based on the in vitro potency differences between human skin keratinocytes and lung fibroblasts to generate an estimated adjusted C_{eff} (C_{eff,adj}) for lung fibrosis.

In a cynomolgus monkey itch study, vixarelimab was used to inhibit scratching behavior, interpreted as a sign of pruritus, induced by a single intradermal administration of recombinant human (rh) IL-31. Supraphysiologic intradermal challenge doses of rhIL-31 between 3 and 24 μg/kg were tested and all induced scratching, with 3 μg/kg resulting in a robust response with minimal variability. Administration of a single IV vixarelimab dose (1, 3, or 10 mg/kg) resulted in dose-and time-dependent reduction in rhIL-31-induced scratching. Higher serum concentrations of vixarelimab resulted in longer scratching inhibition, and duration of effect helped establish 5-8 μg/mL as the serum concentration threshold for vixarelimab efficacy in this model system.

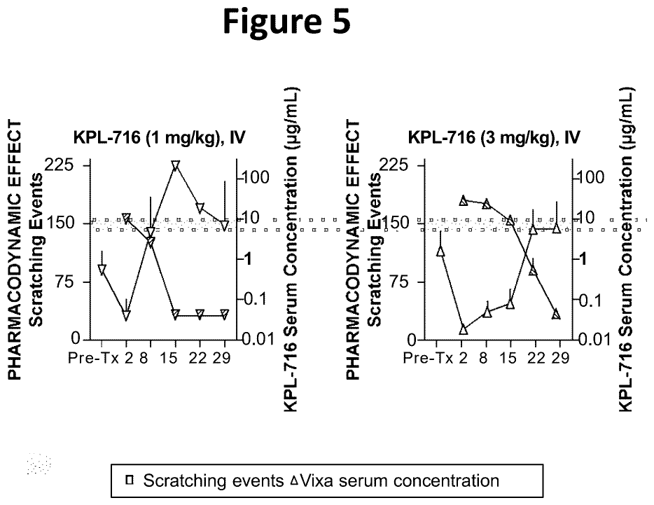

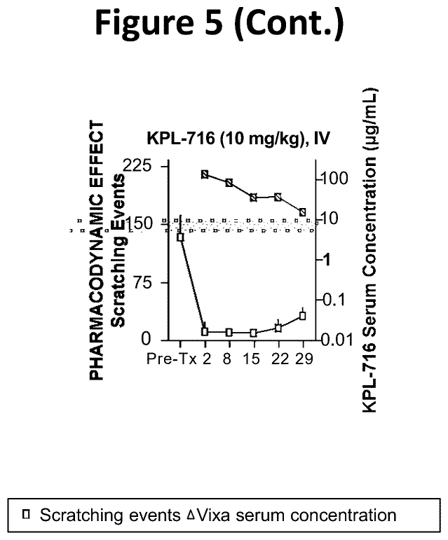

Repeated subcutaneous administration of vixarelimab, 1 mg/kg weekly, 3 mg/kg biweekly, or 8 mg/kg monthly, demonstrated prolonged and significant reduction of IL-31-induced scratching behavior. Monkeys were challenged with rhIL-31 by intradermal injection at various time points following vixarelimab injection. Scratching events following hIL-31 challenge were reported for each group. Concentrations of vixarelimab at the same dosing regimens were simulated and correlated with reduction in rhIL-31-induced scratching, verifying a C_{eff} threshold of 5-8 μg/mL for inhibiting pruritic responses in this model (See FIG. 5).

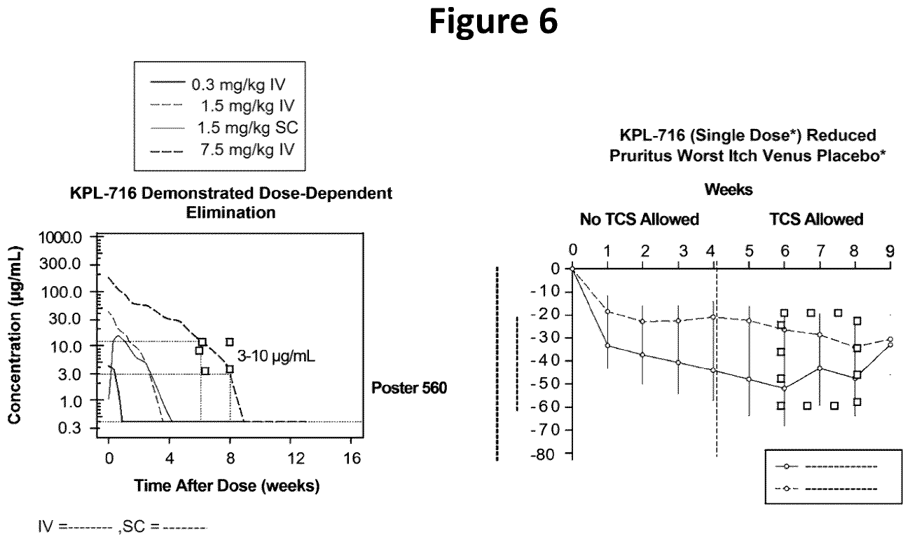

The human Phase 1b clinical study of vixarelimab treatment of AD involved IV administration of vixarelimab at doses of 0.3 mg/kg, 1.5 mg/kg, 7.5 mg/kg, 10 mg/kg or 20 mg/kg and SC administration at doses of 1.5 mg/kg or 360 mg. Patients were monitored for safety as well as for disease severity, pruritis intensity, and quality of life measures including sleep quality. It was found that a sustained efficacy lasted 6-8 weeks following a single IV dose of 7.5 mg/kg in patients with AD, supporting the C_{eff} of 5-8 μg/mL that was identified in the cyno study above (see FIG. 6).

Independently, an in vitro potency study was performed in order to compare the ability of vixarelimab to inhibit OSM-induced STAT3 phosphorylation in human primary normal lung fibroblasts, IPF-derived lung fibroblasts and normal keratinocytes. This study allowed use of the C_{eff} determined in the cyno and human studies above to be converted into an estimated adjusted C_{eff} (C_{eff,adj}) for use in a phase 2 study of vixarelimab in lung fibrosis indications.

The in vitro potency studies were performed as follows. Five normal donor-derived human primary lung fibroblasts, 5 IPF patient-derived human lung fibroblasts, and 5 normal donor-derived human keratinocytes were purchased from Lonza (Basel, Switzerland). All primary cells were cultured in complete media containing RPMI-1640 with 10% heat-inactivated fetal bovine serum, 2 mM L-glutamine, and 1% penicillin-streptomycin. The cells were seeded at 20,000 cells per well into a 96-well plate (Product No. 3595; Corning; Corning, NY) and incubated overnight in an incubator at 37° C. with 5% CO2. The next day, vixarelimab antibody and OSM (Purification Batch PUR1BY00559; Genentech) were serially diluted 3-fold from a starting final concentration of 50 μg/mL for a total of 10 dilutions in RPMI-1640 complete media. To examine the inhibitory potency against different levels of OSM, normal human primary lung fibroblast and IPF patient-derived fibroblast cells were treated with either 1 or 10 ng/mL OSM (final concentration) in the presence of antibodies, and normal human primary keratinocyte cells were treated with either 10 or 100 ng/mL OSM (final concentration) in the presence of antibodies. Forty microliters of serially diluted vixarelimab antibody and 40 μL OSM were mixed and incubated for 10 minutes at room temperature. Then 50 μL of the mixture was added to each well of the assay plate. The assay plate was incubated at 37° C. for 15 minutes. After incubation, STAT3 phosphorylation was measured using the Phospo-STAT3 (Tyr705) kit (Catalog No. K150SVD-4; Meso Scale Discovery [MSD]; Gaithersburg, MD). Cell culture media were removed from the plate, and 60 μL of lysis buffer containing phosphatase and protease inhibitors was added. After 1 hour of incubation at 4° C., 25 μL of cell lysate was transferred using a Biomek i5 Automated Workstation (Beckman Coulter; Indianapolis, IN) to an MSD plate that was previously blocked and washed. The cell lysate was incubated at 4° C. overnight on a shaker. Then the plate was washed 3 times with 200 μL per well of Tris buffer, followed by the addition of 25 μL SULFO-TAG™ labeled anti-phospho-STAT3 detection antibody into each well. After 1 hour of incubation at room temperature on a shaker, the plate was washed 3 times with 200 μL Tris buffer per well, and 150 μL surfactant-based read buffer was added to each well before the plate was read on an MSD MESO SECTOR S 600 instrument. The percentage of phosphorylated STAT3 (pSTAT3) inhibition (% inhibition) for each treatment condition was calculated using the following equation, where the maximum is the MSD signal of OSM only and the minimum is the MSD signal of RPMI-1640 media only:

$$pSTAT 3 ( \% inhibition ) = [ 1 - ( MSD signal - minimum ) \div ( maximum - minimum ) ] \times 100$$

The pSTAT3 (% inhibition) was plotted as a function of antibody concentrations, and the data were fitted to a sigmoidal 4-parameter logistic (4PL) model using Prism (GraphPad; La Jolla, CA). The 50% inhibitory concentration (IC₅₀) value for each donor was determined as the concentration reaching 50% inhibition of maximum activity. The concentration that leads to 90% maximal inhibitory response (90% inhibitory concentration; IC90) was calculated using modeled parameters.

The results show that vixarelimab consistently inhibits OSM-induced STAT3 phosphorylation across a panel of human donor-derived primary cells, including normal lung fibroblasts, IPF-derived lung fibroblasts, and normal keratinocytes. The mean and standard deviation of IC₅₀ and IC₉₀ values were determined by the concentration-response curve fitted to a sigmoidal 4PL model. The results are summarized in Table 3 below.

TABLE 3

| | | Mean of | Standard | Mean of | Standard |
|---|---|---|---|---|---|
| | [OSM] | IC₅₀ | deviation | IC₉₀ | deviation |
| Cell Type | (ng/mL) | (ng/mL) | of IC₅₀ | (ng/mL) | of IC₉₀ |
| Normal lung | 1 | 13.04 | 7.36 | 135.31 | 40.67 |
| fibroblasts | | | | | |
| Normal lung | 10 | 88.17 | 39.7 | 948.07 | 593.49 |
| fibroblasts | | | | | |
| IPF-derived lung | 1 | 12.45 | 4.05 | 114.26 | 45.58 |
| fibroblasts | | | | | |
| IPF-derived lung | 10 | 56.11 | 15.61 | 347.52 | 152.39 |
| fibroblasts | | | | | |
| Normal | 10 | 4.95 | 3.31 | 234.99 | 158.41 |
| keratinocytes | | | | | |
| Normal | 100 | 45.05 | 32.44 | 1038.62 | 948.82 |
| keratinocytes | | | | | |

The results of the in vitro study were used to convert the C_{eff} of 8 μg/mL to a C_{eff,adj} of 20.4 μg/ml for lung fibrosis, using a difference in in vitro IC₅₀ for OSM-induced pSTAT3 activation between keratinocyte and lung fibroblast.

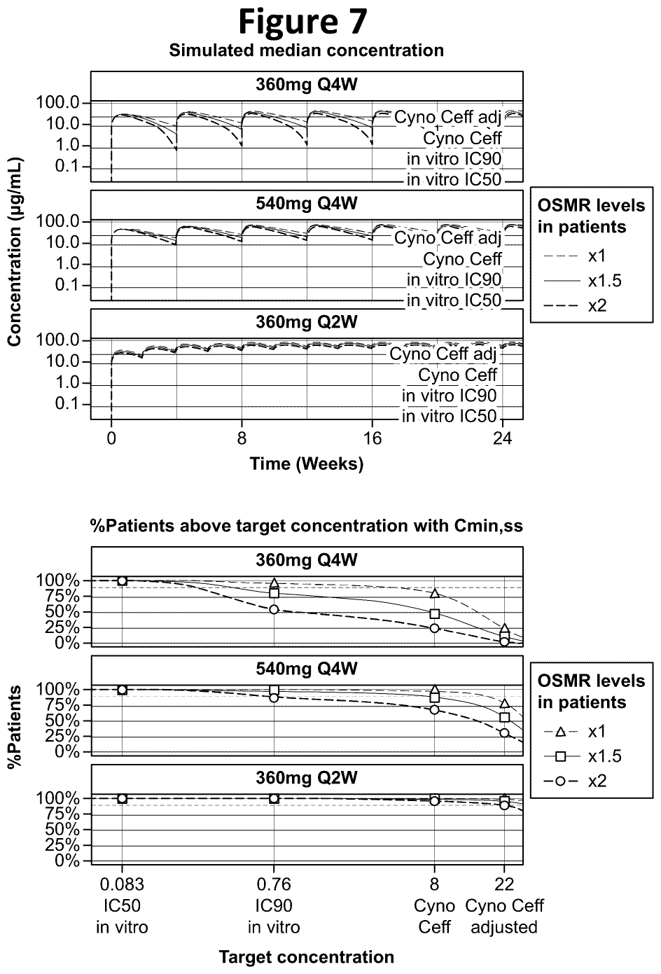

Next, the PK profile of vixarelimab was simulated to estimate the Cₘᵢₙ coverage of the estimated C_{eff,adj} at various dose levels. Simulation was performed with a preliminary target-mediated drug disposition (TMDD) population PK model developed with available clinical PK data from healthy subjects, and AD and PN patients. The PK profiles across healthy subjects and AD and PN patient populations appeared comparable; however, the simulation incorporated an assumed worst-case increase in target amount by 2-fold in IPF patients compared with healthy subjects. The simulated PK profiles are illustrated in FIG. 7.

This simulation showed that over 90% of IPF patients are predicted to have C_{min,SS} above the estimated C_{eff,adj} following administration of 360 mg Q2W, irrespective of the assumed OSMRβ levels in IPF patients. Less frequent dosing was also explored, but simulation results did not show sufficient coverage of the C_{eff,adj}. Specifically, a dosing regimen of 360 mg Q4W provides coverage of <25% of patients above C_{eff,adj}. A dosing regimen of 540 Q4W provides coverage of patients above C_{eff,adj} for >50% of patients with up to a 1.5-fold OSMRβ level increase, but this coverage drops to less than 50% if it is assumed systemic OSMRβ levels increase to 2-fold. These data support the selection of 360 mg Q2W dosing for IPF and SSc-ILD patients to provide target coverage for majority of the patient population.

### Example 4. Phase 2 Study to Evaluate Efficacy, Safety, and PK in IPF and SSc-ILD

A two-cohort, Phase II, multicenter, randomized, double-blind, parallel-group, placebo-controlled study was designed to evaluate the efficacy, safety, and pharmacokinetics of vixarelimab in patients with IPF (Cohort 1) and in patients with SSc-ILD (Cohort 2). Cohort 1 will enroll about 200 patients with IPF (of whom up to about 50 may be on concurrent standard-of-care anti-fibrotic therapy), and Cohort 2 will enroll about 60-120 patients with SSc-ILD (of whom up to about 30 may be on concurrent standard-of-care anti-IL-6 therapy and up to about 30 patients may be on concurrent standard-of-care nintedanib therapy). Each cohort will be analyzed separately. Up to approximately 290 patients with IPF and SSc-ILD may be enrolled in the OLE portion of the study.

Eligible patients will have n FVC (forced vital capacity) ≥ 45% predicted and a forced expiratory volume in 1 second (FEV1)-to-FVC ratio >0.70; a subset of patients will be receiving stable standard-of-care treatment for their disease.

In addition, patients in Cohort 1 will be aged 40-85 years and have a documented diagnosis of IPF or IPF (likely) per the ATS/ERS/JRS/ALAT guidelines (Raghu et al. 2022). Patients with a clinical context suggestive of IPF and with a high-resolution computed tomography (HRCT) pattern of usual interstitial pneumonia (UIP) or probable UIP are considered to have a diagnosis of IPF when biopsy is not available (Raghu et al. 2022). Patients will have an HRCT pattern consistent with the diagnosis of IPF, confirmed by central review of chest HRCT and central review of any available lung biopsy. For patients receiving pirfenidone or nintedanib treatment for IPF, they will have been treated for ≥3 months with a stable dose for ≥4 weeks prior to screening and during screening, with plans to continue treatment during the study period. For patients not currently receiving nintedanib or pirfenidone treatment, such patients are treatment naïve or have discontinued such treatment ≥4 weeks prior to screening and during screening with no plans to start or restart therapy during the study period.

Patients in Cohort 2 will be aged 18-85 years and have an initial documented diagnosis of systemic sclerosis (SSc) as defined using the American College of Rheumatology/EULAR criteria (van den Hoogen et al. 2013), an HRCT pattern demonstrating ≥10% fibrosis, and evidence of progressive pulmonary fibrosis, defined as at least two of the following criteria occurring within the past year with no alternative explanation (Raghu et al., 2022): worsening respiratory symptoms, and physiological evidence of disease progression by either of: absolute decline in FVC ≥5% predicted within 1 year of follow-up; absolute decline in CL_{CO} (corrected for hemoglobin) ≥10% predicted within 1 year of follow-up. Patients will have radiological evidence of disease progression per ATS/ERS/JRS/ALAT 2022 guidelines (Raghu et al. 2022). For patients receiving anti-IL-6 (e.g., tocilizumab) treatment for SSc-ILD, they will have had treatment for ≥3 months with a stable dose for ≥4 weeks prior to and during screening and no intention to change or modify their treatment regimen for the duration of the study. Patients not currently receiving anti-IL-6 treatment, such patients are treatment naïve or have discontinued such treatment ≥4 weeks prior to screening and during screening with no plans to start or restart therapy during the study period. For patients treated with permitted standard of care immunosuppressive agents for their underlying skin disease (e.g., mycophenolate-mofetil (MMF), methotrexate (MTX)), they will have had stable treatment dosing for ≥3 months with a stable dose for ≥4 weeks prior to screening no intention to change or modify their treatment regimen for the duration of the study period.

Exclusion criteria for all patients include those with a percentage of predicted FVC value showing improvement in the 6-month period prior to screening and including screening value, known post-bronchodilator response in FEV₁ and FVC (defined as an increase by 12% and 200 mL)

Following a screening period of up to 40 days, eligible patients within each cohort will be randomized 1:1 to receive subcutaneous (SC) injections of vixarelimab 360 mg or placebo Q2W for 26 doses over 52 weeks, followed by a follow-up visit approximately 9 weeks after the final dose. Randomization in Cohort 1 will be stratified by concomitant anti-fibrotic treatment and region, and randomization in Cohort 2 will be stratified by concomitant anti-IL-6 treatment.

### Assessments

Patients will return to the clinic every 2 weeks until the treatment completion visit at Week 52 for administration of vixarelimab and for assessment of vital signs, adverse events, and concomitant medications, and every 4 weeks for spirometry (e.g., FVC and (FEV1)-to-FVC ratio), assessment of healthcare utilization, assessment of ILD exacerbations and hospitalizations, physical examinations, and certain laboratory tests. Other assessments, including 6MWT (6-minute walk test), DL_{CO}, and patient reported outcomes (PROs), will be performed less frequently. HRCT (high-resolution computed tomography) will be performed at screening (if an available HRCT of acceptable quality within 3 months prior to randomization is not available), at Week 12, and at Week 52, and samples for PK and ADA analysis will be collected throughout the study. In addition, patients in Cohort 2 will be required to provide a skin biopsy at baseline and at Week 52 and will be assessed using the mRSS at various time points during the study. For patients at participating sites after Week 4, study drug may be administered every other time point by a trained nursing professional at the patient's home or another suitable location if the patient has given written informed consent to participate in mobile nursing (MN) visits.

On dosing days, dosing will occur after all safety and efficacy assessments scheduled for that visit are complete. Patients will return to the clinic every 4 weeks until the treatment completion visit at Week 52 for assessments including vital signs, spirometry (e.g., FVC and (FEV1)-to-FVC ratio), 6MWT, and PROs. HRCT will be performed at screening (if an available HRCT of acceptable quality within 3 months prior to randomization is not available), at Week 12, and at Week 52, and samples for PK and ADA analysis will be collected throughout the study. In addition, patients in Cohort 2 will be required to provide a skin biopsy at baseline and at Week 52 and will be assessed using the mRSS at various time points during the study.

Patients (including those receiving placebo) who complete the 52-week treatment period will be invited to enroll in an open-label extension (OLE) study to receive treatment with vixarelimab at the same dose and schedule for up to 1 year. Patients who do not enroll in the OLE study will return to the clinic for a follow-up assessment approximately 9 weeks after their final dose (i.e., at Week 59) to ensure safety.

For both cohorts, the primary endpoint is the absolute change from baseline to Week 52 in FVC (mL), and the key secondary endpoint is the change in 6MWT distance in meters at Week 52. Other secondary endpoints include absolute change in baseline to Week 52 in percentage of predicted FVC; change from baseline to Week 52 in DL_{CO}[Hb]; time to disease progression, defined as time to first occurrence of ≥10% absolute decline in percentage of predicted FVC, ≥15% relative decline in 6MWT distance, lung transplantation, or death; time to first acute exacerbation of ILD, or suspected acute exacerbation of ILD, as determined by the clinical adjudication committee (CAC); change from baseline to Week 52 in quantitative lung fibrosis on high-resolution computed tomography (HRCT) scan of the thorax; and survival, as measured by all-cause mortality.

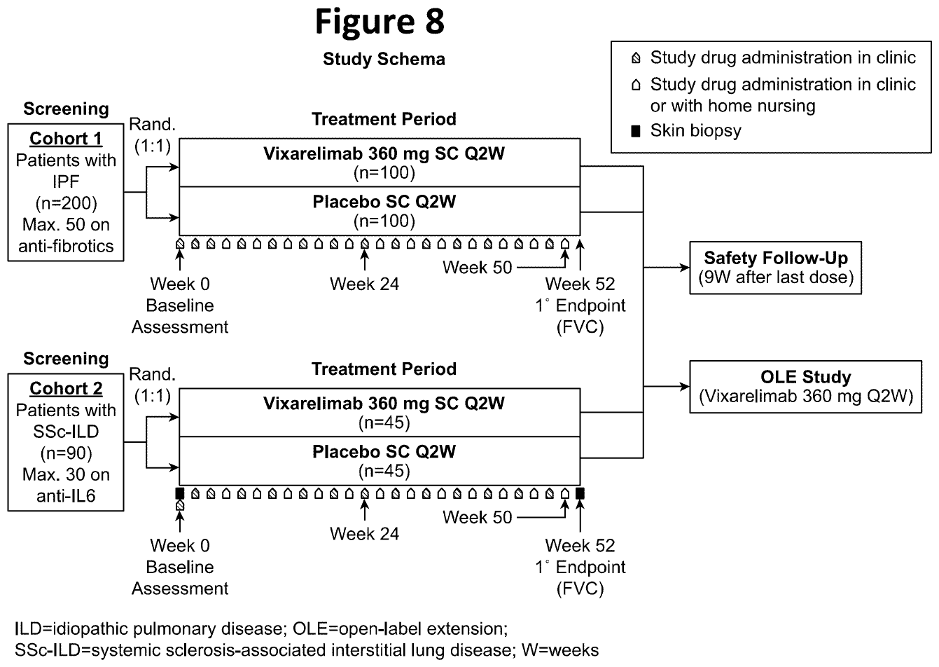

Patients who do not meet the criteria for participation in this study (screen failure) may qualify for one re-screening opportunity (for a total of two screenings per patient) at the investigator's discretion. In addition, if a patient fails a test due to technical issues with the test (e.g., laboratory sample hemolyzed and not able to be analyzed), the patient will be allowed to repeat the test if still within the screening period. The investigator will maintain a record of reasons for screen failure. The study design can be found in FIG. 8.

### Open-Label Extension Study

An open-label extension (OLE) study will be performed, subject to approval by local Institutional Review Boards or Ethics Committees (IRBs/ECs) and relevant health authorities. Patients in both Cohort 1 and Cohort 2 who complete the Phase 2 study (Example 4) treatment period through Week 52 will be given the option to enroll in the OLE study and receive treatment with open-label vixarelimab, if eligible and provided that the OLE study is open in their respective country.

Patients should start the OLE period on the same day as the Week 52 visit in the double-blinded treatment period after completing all required Week 52 assessments. Alternatively, the first dose in the OLE period may be administered up to 4 weeks (+5 days) after the last dose of study drug in the double-blinded treatment period. The first visit of the OLE period will be considered the OLE baseline. Patients will return to the clinic for scheduled visits to receive an additional 52 weeks of open-label vixarelimab 360 mg SC Q2W and for assessments including vital signs, spirometry, and 6MWT until the OLE treatment completion visit. Patients will return to the clinic for a follow-up visit approximately 9 weeks after the final dose.

For each cohort in the OLE study, treatment efficacy will be determined by measuring absolute change from OLE baseline to OLE Week 52 in FVC (mL); absolute change from OLE baseline to OLE Week 52 in 6MWT distance (in meters); absolute change from OLE baseline to OLE Week 52 in percentage of predicted FVC; change from OLE baseline to OLE Week 52 in DL_{CO}[Hb]; change from OLE baseline to OLE Week 52 in quantitative lung fibrosis on HRCT scan of the thorax; and survival, as measured by all-cause mortality. For Cohort 2 only, change from OLE baseline to OLE Week 52 in skin sclerosis, as measured by the modified Rodnan skin score (mRSS).

### Example 5. IL-6 drives CD64+ Macrophage Activation in Murine Pulmonary Inflammation and Fibrosis

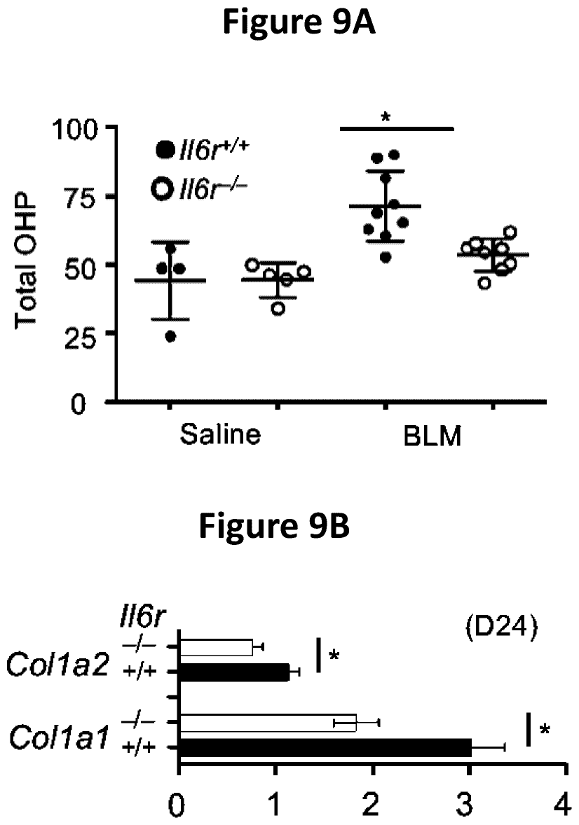

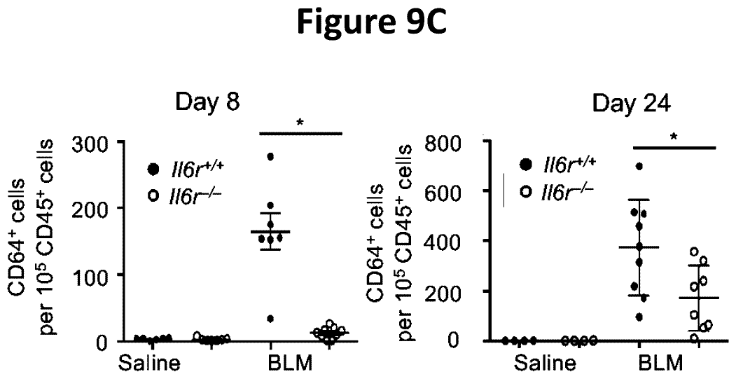

The role of IL-6 in the murine bleomycin (BLM)-induced lung injury, inflammation and fibrosis model was investigated. IL-6 receptor had knockout mice (Il6r−/− mice) had reduced lung injury and inflammation at both day 8 and day 24 post-BLM (data not shown) with reduced hydroxyproline, an amino acid necessary for collagen biosynthesis (FIG. 9A) along with reduced Col1a1 and Col1a2 gene expression in the lung (FIG. 9B). Correlating with reduced disease in Il6r−/− mice was a reduction in the proportion and total number of CD64+ macrophages (CD45+CD11c+SiglecF−MHCII+CD11b+CD64+) at both day 8 and day 24 post-BLM (FIG. 9C), suggesting that IL-6 contributes to macrophage recruitment.

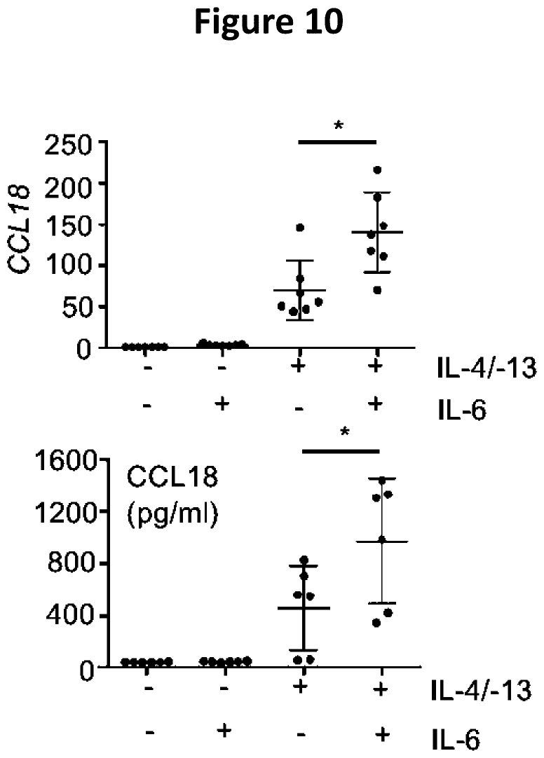

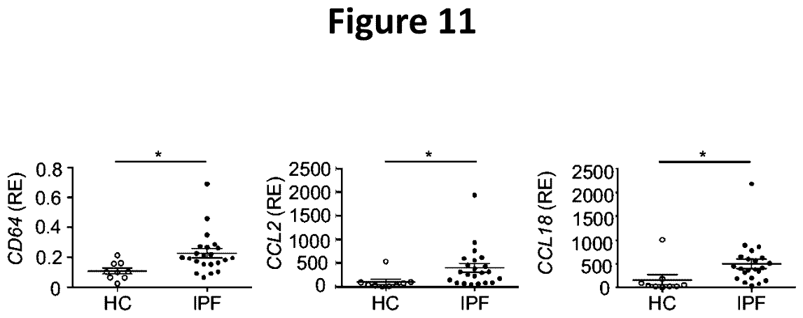

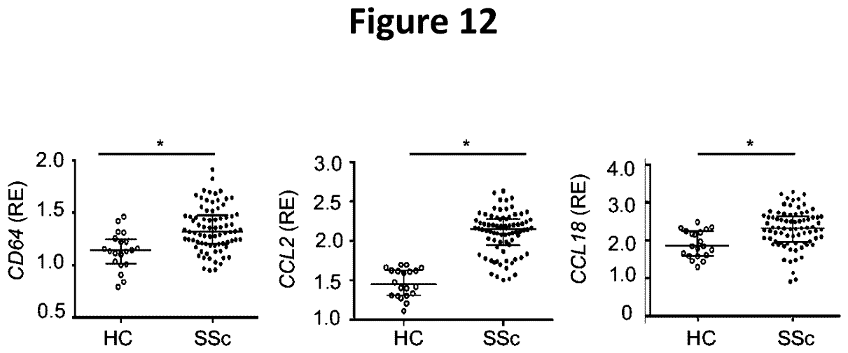

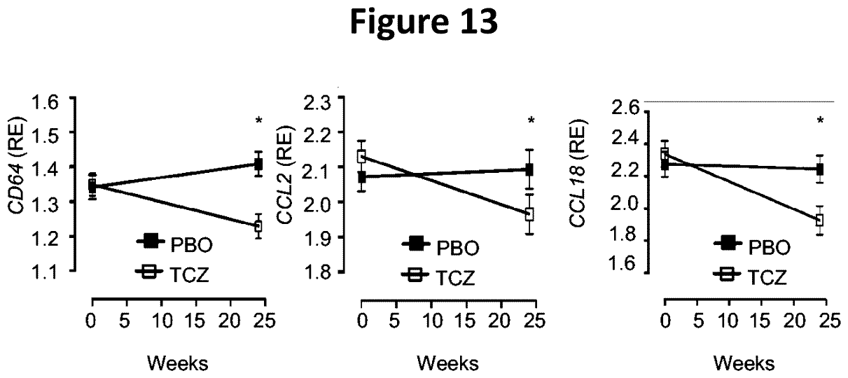

To determine whether these observations in murine macrophages translated to human macrophages, we primed human monocyte-derived macrophages with IL-4/13 and treated them with IL-6. As expected, IL-6 significantly increased CCL18 transcripts and secretions (FIG. 10), a chemokine known to be prognostic for worse ILD. IPF and SSc-ILD patient samples demonstrated that IL-6-regulated macrophage genes CD64, CCL2 and CCL18 were all significantly increased in the lung tissue of IPF (FIG. 11) or skin of SSc-ILD (FIG. 12) patients. Following 24 weeks of anti-IL-6R mAb (tocilizumab) treatment in the clinic, there was a very clear and strong pharmacodynamic effect of these genes in the skin of SSc-ILD patients in a separate phase 2 clinical trial (FIG. 13), suggesting that IL-6-regulated macrophage activation in these ILD patients. Thus, IL-6 may contribute to lung function decline in patients with ILD via an inflammatory macrophage mediated activation pathway, and that IL-6 and potentially macrophage-independent pathways may contribute to progressive fibrosis in ILD.

### Example 6. OSMR-Dependent Pathogenesis in Human Disease-Relevant Cells

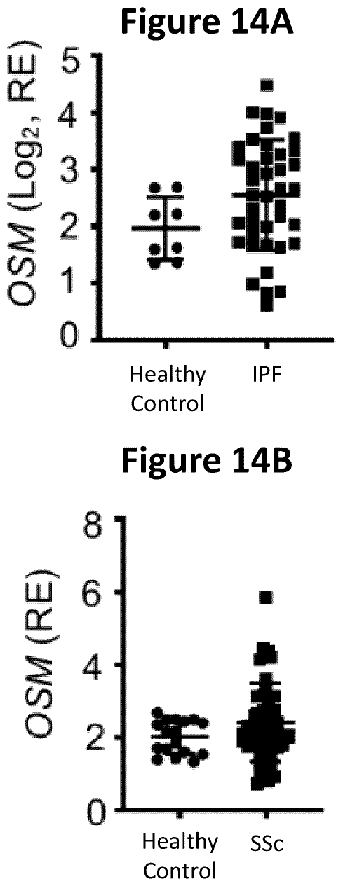

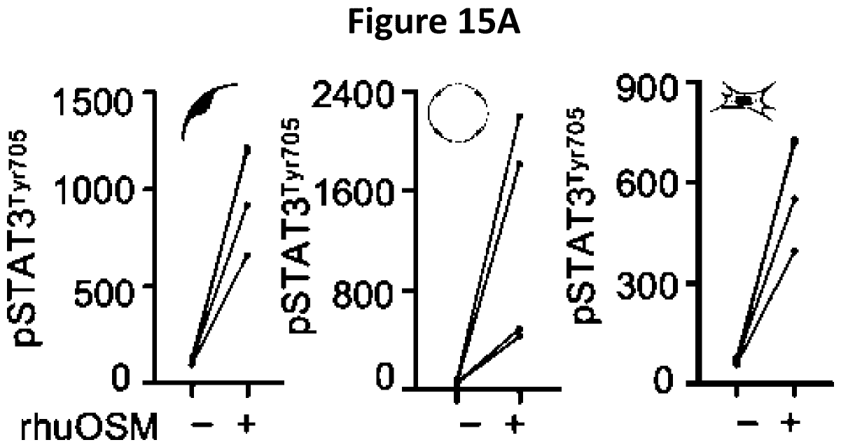

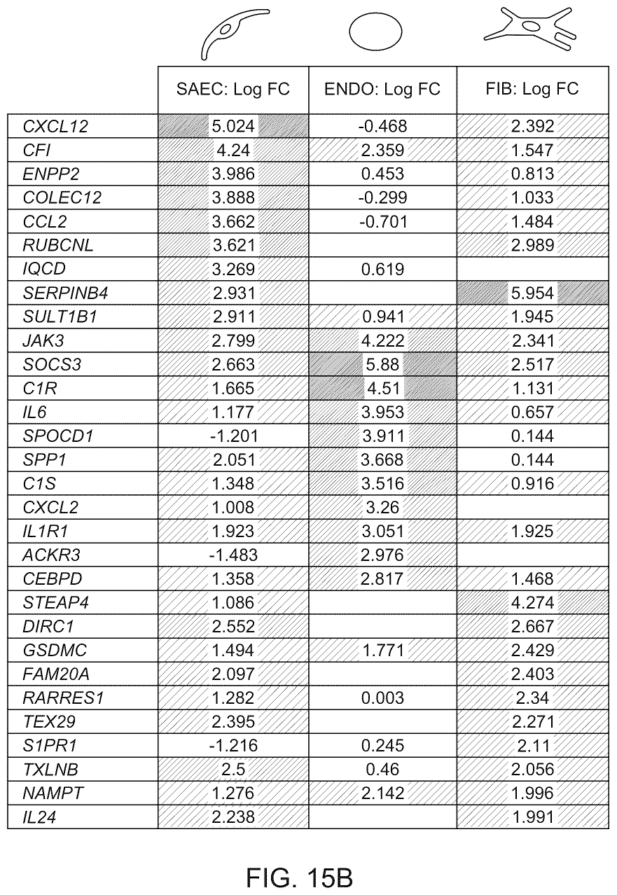

To establish the role of OSMR in the pathogenesis of IPF and SSc, lung and skin biopsies were obtained from healthy patients and from patients with IPF or SSc. RNA-seq analysis of those biopsies demonstrated that OSM was elevated in the lung tissue and skin from IPF (FIG. 14A) and SSc (FIG. 14B). Primary human SAEC, endothelial cells and fibroblasts in vitro were cultured and stimulated with recombinant human OSM (10ng/ml) for 15 mins. Cell lysates were recovered and pSTAT3T^{yr705} was measured by Meso Scale Discovery (MSD) assays. All three primary human cell types responded to OSM with significant STAT3 phosphorylation, whether cells were derived from healthy or IPF patients (FIG. 15A). A comparative transcriptional analysis of these 3 cell types following 24 hrs of exposure to OSM was performed. Of the top 10 OSM-induced transcripts in each cell type, many were commonly upregulated across all cell types (CFI, JAK3, SOCS3, CIR, SPP1, IL1R1, CEBPD, GSDMC, NAMPT), with some notable cell-specific responses observed, including OSM-induced ENNP2 (Autotaxin) in SAEC, OSM-induced IL6 in endothelial cells and OSM-induced S1PR1 in fibroblasts, all of which have clear roles in pulmonary fibrosis (FIG. 15B).

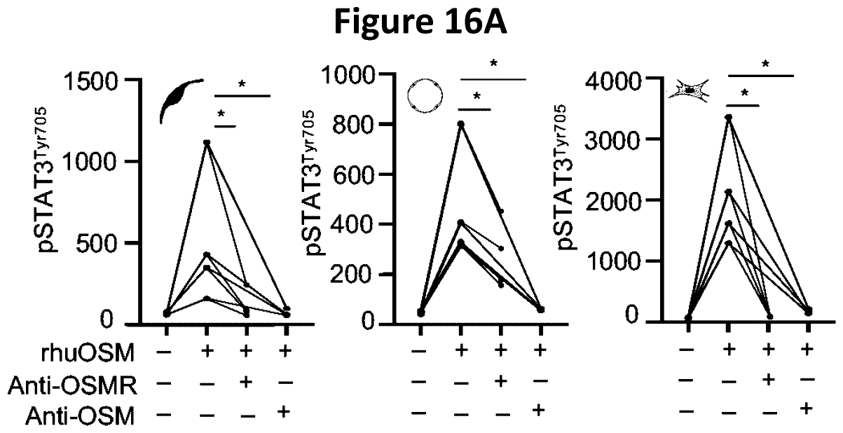

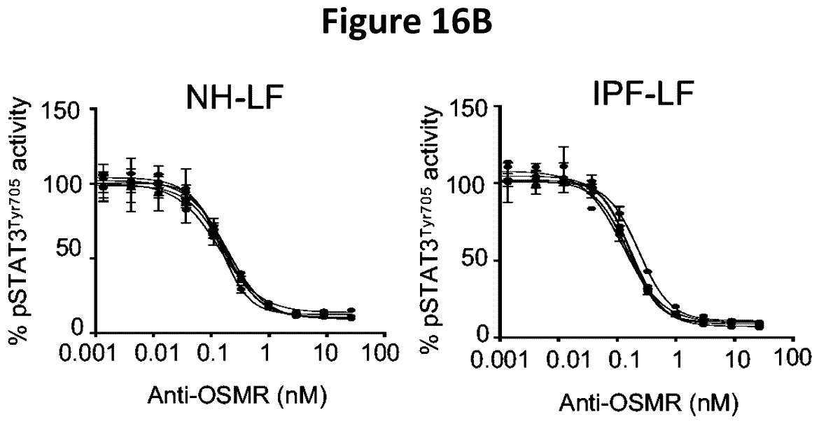

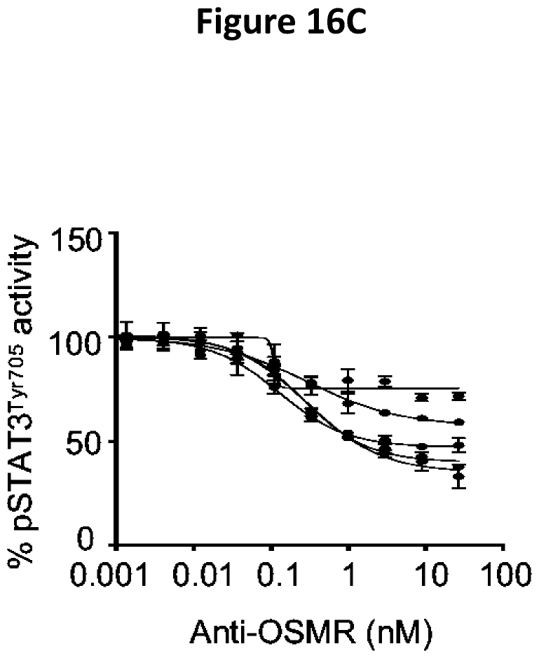

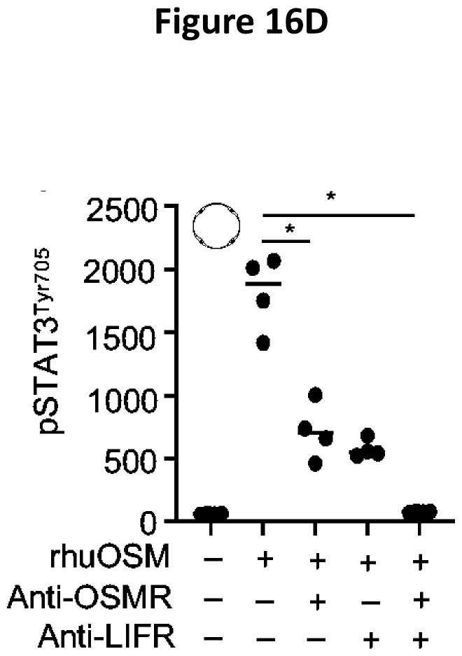

OSM binds to gp130 which then heterodimerizes with either OSMR or LIFR for signal transduction. To determine whether OSM-driven responses were mediated via OSMR or LIFR, a test to determine whether antagonizing OSMR alone was sufficient to block OSM-driven responses in these 3 OSM-responsive and disease-relevant cell types was conducted. Using a newly generated anti-human OSMR blocking Ab, OSM-induced pSTAT3 was almost completely inhibited in SAEC and fibroblasts (FIG. 16A), whether derived from healthy or IPF donors (FIG. 16B). The OSMR blocking Ab inhibited OSM-induced pSTAT3 in endothelial cells by approximately 50% (FIG. 16A and 16C). The addition of anti-LIFR confirmed that OSM uses both OSMR and LIFR for signaling in endothelial cells with complete inhibition obtained when both mAbs were used (FIG. 16D). These data indicate that OSMR antagonism alone is sufficient to almost completely mitigate OSM-driven pSTAT3 in fibroblasts and epithelial cells.

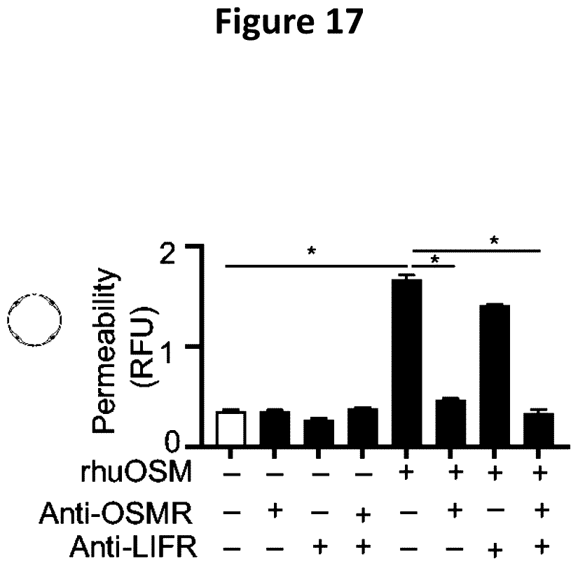

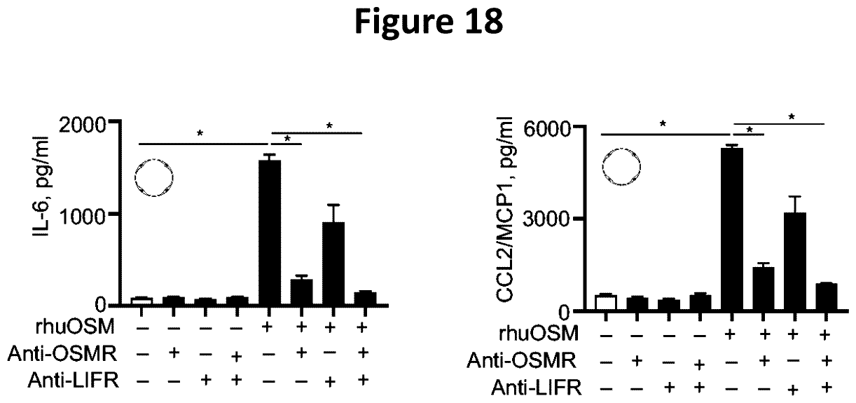

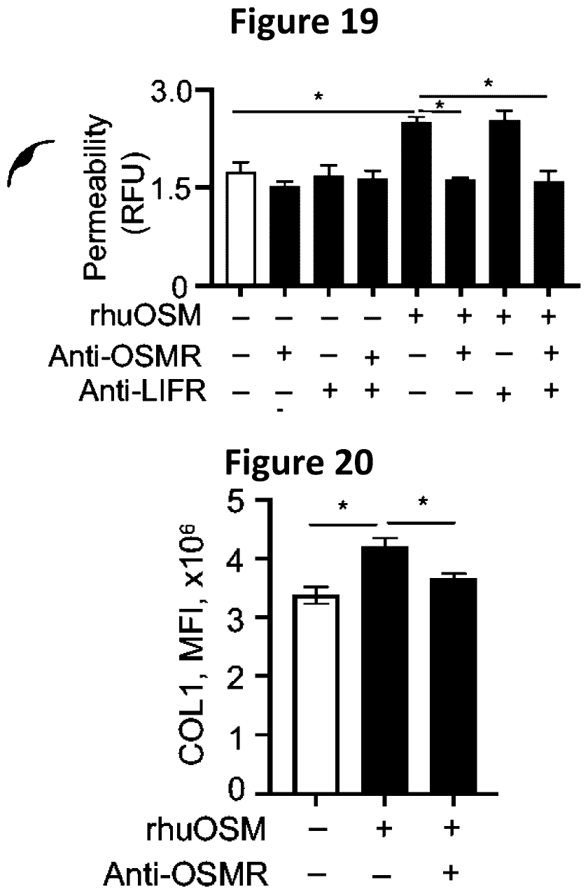

It was next determined whether OSM might cause lung endothelial cell damage and whether this was dependent on OSMR or LIFR. Indeed, OSM-induced endothelial cell disruption and permeability, compromising barrier integrity, which could be completely prevented with anti-OSMR antagonism (FIG. 17). Anti-LIFR mAb treatment had little to no impact on OSM-induced permeability. Similarly, OSM-induced IL-6 and CCL2/MCP1 secretion from lung endothelial cells was largely dependent upon OSMR rather than LIFR (FIG. 18). Thus, despite OSM-induced pSTAT3 was only partially mediated by OSMR, endothelial cell permeability and inflammatory cytokine production was dominantly mediated by OSMR. Similar studies were carried out with primary lung SAEC grown in 3D organoids. Similar to endothelial cells, OSM could also disrupt SAEC integrity with a significant increase in permeability (FIG. 19). OSM-driven SAEC permeability was also dependent upon OSMR and not LIFR, in line with pSTAT3 data (FIG. 16A). Taken together these data indicate that OSM can disrupt both epithelial and endothelial cell integrity, a potential pathogenic axis in ILD, and that this process is dependent upon OSMR signaling.

Most strikingly, OSM directly induced collagen (COL1) secretion from primary human fibroblasts. This process was also OSMR-dependent (FIG. 20), giving direct mechanistic evidence for a potential role of OSM in human fibrotic diseases.

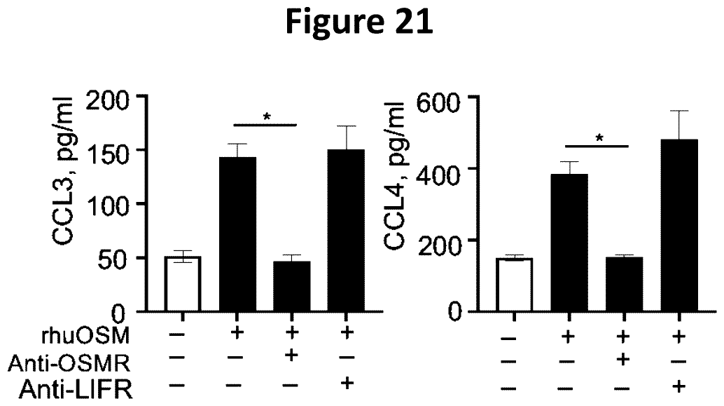

To determine whether OSMR is also required for OSM-driven responses in multi-cellular human lung explants, we stimulated precision cut lung slices (PCLS) with OSM and treated these cultures with anti-OSMR or anti-LIFR blocking Abs. In agreement with the primary human monoculture systems, OSM-driven chemokine production (CCL3 and CCL4) from PCLS were dependent upon OSMR, and not LIFR (FIG. 21), supporting the therapeutic targeting of OSMR to prevent OSM activity in human lung diseases. Collectively these studies provide the biological rationale and mechanistic data to support the therapeutic development of an OSMR antagonist to treat fibrotic lung diseases. The proposed benefit of OSMR antagonism may be further enhanced with the combined treatment with IL6R antagonists.

### Example 7. Combined IL6 and OSM Antagonism Reduces Lung Injury, Inflammation and Fibrosis

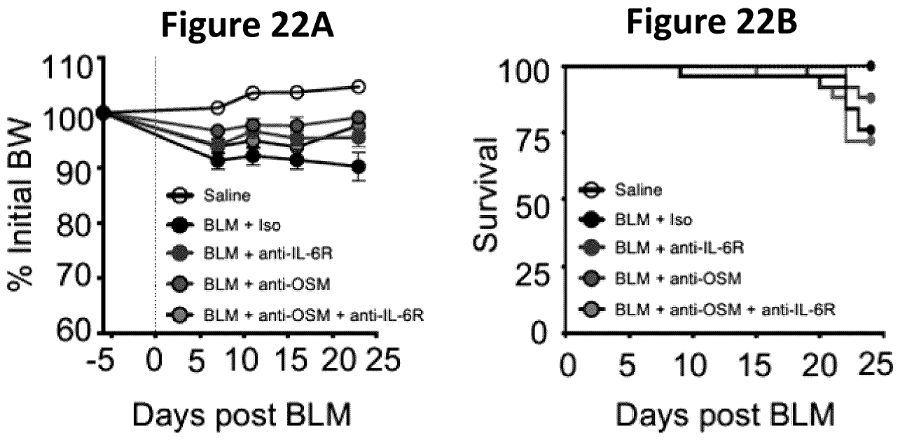

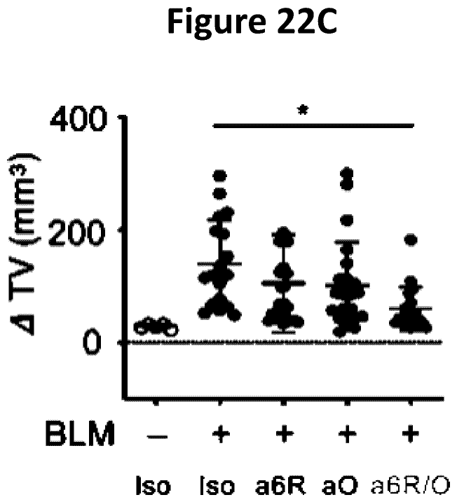

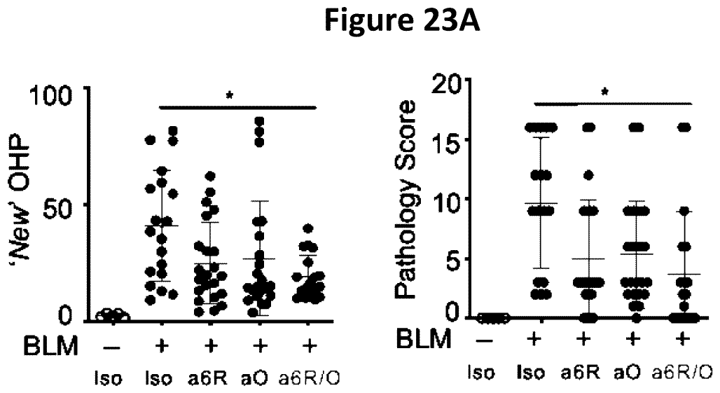

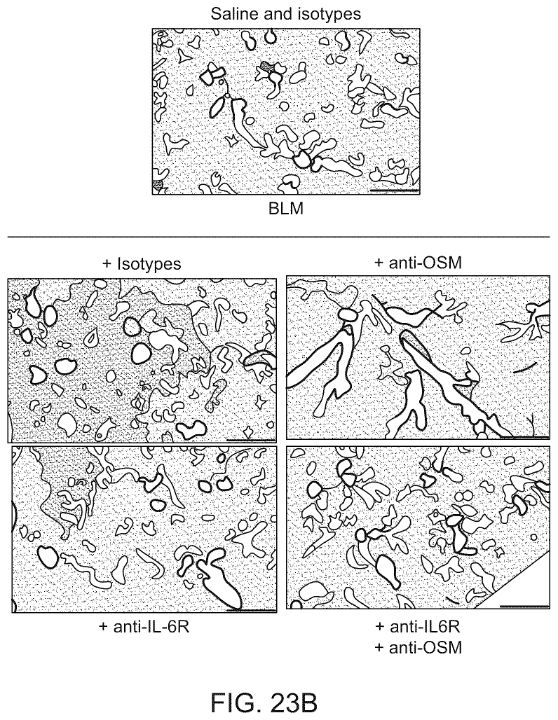

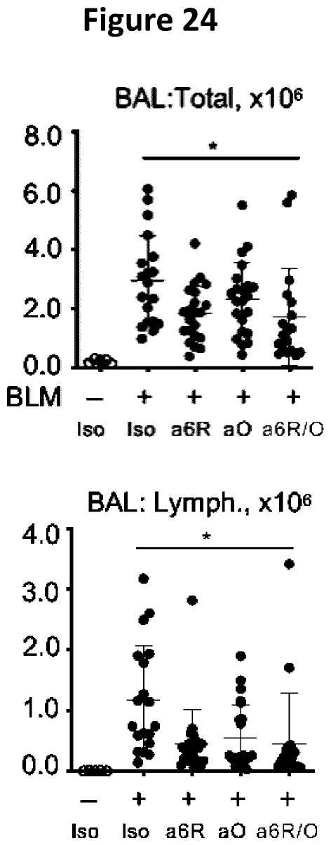

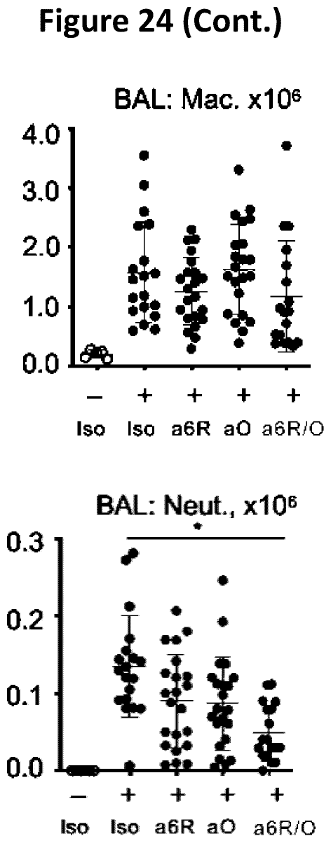

Mice were treated with anti-IL-6R mAbs, a murine surrogate to tocilizumab, anti-OSM mAbs, or a combination of both mAbs. BLM-exposed mice, irrespective of Ab treatment, lost weight (FIG. 22A) with a small proportion of mice succumbing to BLM-induced disease (FIG. 22B). Both anti-IL-6R or anti-OSM treatment reduced lung damage, determined by changes in tissue volume (TV) (FIG. 22C). The combination of anti-IL-6R and anti-OSM reduced tissue volume by approximately 60% (BLM+Iso, 140±18.3 mm3 compared to BLM+aIL6/OSM, 60.2±9.2 mm3). Effects of the combo treatment extended to fibrotic endpoints, hydroxyproline measurements and pathology scores (FIG. 23A and FIG. 23B). Furthermore, using airway infiltrates as a surrogate for inflammation, anti-IL-6R treatment reduced inflammation, but a greater effect especially for reducing airway neutrophils, was observed in mice given both anti-IL-6R+anti-OSM (FIG. 24). These data indicate that IL-6 and OSM contribute to BLM-induced lung injury, inflammation, and fibrosis in a non-overlapping manner.

Although the foregoing disclosure has been described in some detail by way of illustration and example for purposes of clarity of understanding, the descriptions and examples should not be construed as limiting the scope of the disclosure. The disclosures of all patent and scientific literature cited herein are expressly incorporated in their entirety by reference.

1. A method for treating a pulmonary fibrotic disease comprising administering to a subject in need thereof a therapeutically effective dose of an anti-OSMRβ (oncostatin M receptor beta) antibody.

2. The method of claim 1, wherein the pulmonary fibrotic disease is selected from the group consisting of progressive pulmonary fibrosis (PPF), idiopathic pulmonary fibrosis (IPF), and systemic sclerosis-interstitial lung disease (SSc-ILD).

3. The method of claim 1, wherein the anti-OSMRβ antibody inhibits signaling of the type II OSMR by OSM and IL-31.

4. The method of claim 1, wherein the anti-OSMRβ antibody is vixarelimab.

5. The method of claim 1, wherein the therapeutically effective dose is about 360 mg to 720 mg of the anti-OSMRβ antibody.

6. The method of claim 1, wherein the administering comprises administering the therapeutically effective dose once per week, once every 2 weeks, once every 3 weeks, once every 4 weeks, or once every month.

7. The method claim 1, wherein the administering comprises administering the therapeutically effective dose subcutaneously or intravenously.

8. The method of claim 1, wherein prior to treatment with the anti-OSMRβ antibody the subject has:
(a) a percentage of predicted forced vital capacity (% FVC) of about 35% to 90%, about 35% to 75%, about 35% to 50%, about 45% to 55%, about 30% to 60%, about 50% to 90%, about 50% to 75%, about 40% to 45%, about 40% to 50%, about 45% to 50%, or about 45% to about 50%; or
(b) a forced expiratory volume in 1 second (FEV1)-to-FVC ratio of about 0.35 to 0.70, about 0.50 to 0.70, about 0.60 to 0.70, about 0.35 to 0.50, about 0.40 to 0.50, about 0.50 to 0.60, about 0.60 to 0.70, about 0.70 to 0.80.

9. The method of claim 1, wherein the administering the dose of anti-OSMRβ antibody to the subject results in:
(a) a change in FVC in the subject, wherein the change is a measure of absolute change in FVC in milliliters (ml) over a treatment period beginning the time at the first administration of the anti-OSMRβ antibody until the time at which a later dose of the anti-OSMRβ antibody is administered, optionally wherein the change in FVC during the treatment period is a decrease in FVC of less than 25 mL, 50 mL, 75 mL, 100 mL, 125 mL, 150 mL, 175 mL, or 200 mL, 225 mL or 250 mL or an increase in FVC of at least 25 mL, 50 mL, 75 mL, 100 mL, 125 mL, 150 mL, 175 mL, or 200 mL, 225 mL or 250 mL;
(b) an increase in DL_{CO}[Hb] in the subject, wherein the change is a measure of absolute change in DL_{CO}[Hb] over a treatment period beginning at the time of the first administration of the anti-OSMRβ antibody until the time at which a later dose of the anti-OSMRβ antibody is administered, optionally wherein the change in DL_{CO}[Hb] during the treatment period is an increase of at least 5%, 10%, 15%, 20%, 25%, 30%, 35%, 40%, 45%, 50%, 55%, 60%, 65%, 70%, 75%, 80% or 90%;
(c) a change in distance traveled by the subject in the 6-minute walk test (6MWT) wherein the change is a measure of the distance traveled by the subject in the 6MWT over a treatment period beginning at the time of the first administration of the anti-OSMRβ antibody until the time at which a later dose of the anti-OSMRβ antibody is administered, optionally wherein the change in distance during the treatment period is (a) an increase of at least 5%, 10%, 15%, 20%, 25%, or 30%; or (b) a decrease of less than about 5%, 10%, 15%, 20%, 25%, or 30%; or
(d) a change in cough relative to baseline, wherein the change is a measure of the cough frequency over a treatment period beginning at the time of the first administration of the anti-OSMRβ antibody until the time at which a later dose of the anti-OSMRβ antibody is administered, wherein the change is a reduction in cough frequency, and wherein cough is measured by digital continuous ambulatory cough detection device.

10. The method of claim 9, wherein the treatment period is about 6 weeks, about 12 weeks, about 24 weeks, about 36 weeks, about 48 weeks, about 60 weeks, or about 72 weeks.

11. The method of claim 1, wherein the anti-OSMR antibody is administered to the subject in combination with a second therapeutic agent.

12. The method of claim 11, wherein the second therapeutic agent pirfenidone, nintedanib, or an anti-IL-6 receptor antibody.

13. The method of claim 12, wherein the anti-IL-6 receptor antibody comprises a heavy chain comprising the amino acid sequence of SEQ ID NO: 13 and a light a light chain comprising the amino acid sequence of SEQ ID NO: 14.

14. The method of claim 12, wherein the anti-IL-6 receptor antibody comprises the six CDRs of tocilizumab.

15. A method of treating a pulmonary fibrotic disease in a subject in need thereof, the method comprising administering to the subject a therapeutically effective amount of (a) an anti-OSMRβ antibody and (b) an anti-IL-6 receptor antibody.

16. The method of claim 15, wherein the anti-OSMRβ antibody comprises (a) a heavy chain variable domain (VH) comprising SEQ ID NO:7 and a light chain variable domain (VL) comprising SEQ ID NO:8; or (b) a heavy chain (HC) comprising SEQ ID NO:5 and a light chain (LC) comprising SEQ ID NO:6.

17. The method of claim 15, wherein the anti-OSMRβ antibody is vixarelimab.

18. The method of claim 15, wherein the heavy chain of the anti-IL-6 receptor antibody comprises the amino acid sequence of SEQ ID NO: 13, and the light chain of the anti-IL-6 receptor antibody comprises the amino acid sequence of SEQ ID NO: 14.

19. The method of claim 15, wherein the anti-IL-6 receptor antibody is tocilizumab.

20. The method of claim 15, wherein the anti-IL-6 receptor antibody comprises the six CDRs of tocilizumab.

21. The method of claim 15, wherein the anti-OSMRβ antibody is vixarelimab and the anti-IL-6 receptor antibody is tocilizumab.

22. The method of claim 15, wherein the pulmonary fibrotic disease is selected from the group consisting of progressive pulmonary fibrosis (PPF), idiopathic pulmonary fibrosis (IPF), and systemic sclerosis-interstitial lung disease (SSc-ILD), preferably IPF.

23. The method of claim 15, wherein the anti-OSMRβ antibody and the anti-IL-6 receptor antibody are administered: (a) simultaneously; (b) sequentially; (c) in the same composition; or (d) in different compositions.

24. The method of claim 16, wherein the subject is human.

25. The method of claim 1, wherein the subject is human.


SEQUENCE LISTING (14 sequences)

SEQ ID NO: 1 - protein, 452 aa, unidentified
QVQLVQSGAEVKKPGASVKVSCKASGYTFTSYDINWVRQATGQGLEWMGWMNPNSGNTDYAQKFQGRVTMTRNISISTAYIELSSLRSEDTAVYYCARDMVAANTDYYFYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 2 - protein, 216 aa, unidentified
QSVLTQPPSASGTPGQRVTISCSGSSSNVGSNTVSWYQQLPGTAPKLLIYTNNRRPSGVPDRFSGSKSGTSASLAISGLQSEDEADYFCAALDDSLNGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 126 aa, unidentified
QVQLVQSGAEVKKPGASVKVSCKASGYTFTSYDINWVRQATGQGLEWMGWMNPNSGNTDYAQKFQGRVTMTRNISISTAYIELSSLRSEDTAVYYCARDMVAANTDYYFYYGMDVWGQGTTVTVSS

SEQ ID NO: 4 - protein, 111 aa, unidentified
QSVLTQPPSASGTPGQRVTISCSGSSSNVGSNTVSWYQQLPGTAPKLLIYTNNRRPSGVPDRFSGSKSGTSASLAISGLQSEDEADYFCAALDDSLNGVVFGGGTKLTVLG

SEQ ID NO: 5 - protein, 452 aa, unidentified
QVQLVQSGAEVKKPGASVKVSCKASGYTFTSYEINWVRQATGQGLEWMGWMNPNSGYTGYAQKFQGRVTMTRDTSISTAYMEMSSLRSEDTAVYYCARDIVAANTDYYFYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 216 aa, unidentified
QSVLTQPPSASGTPGQRVTISCSGSNSNIGSNTVNWYHQLPGTAPKLLIYNINKRPSGVPDRFSGSKSGSSASLAISGLQSEDEADYYCSTWDDSLDGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 7 - protein, 126 aa, unidentified
QVQLVQSGAEVKKPGASVKVSCKASGYTFTSYEINWVRQATGQGLEWMGWMNPNSGYTGYAQKFQGRVTMTRDTSISTAYMEMSSLRSEDTAVYYCARDIVAANTDYYFYYGMDVWGQGTTVTVSS

SEQ ID NO: 8 - protein, 111 aa, unidentified
QSVLTQPPSASGTPGQRVTISCSGSNSNIGSNTVNWYHQLPGTAPKLLIYNINKRPSGVPDRFSGSKSGSSASLAISGLQSEDEADYYCSTWDDSLDGVVFGGGTKLTVLG

SEQ ID NO: 9 - protein, 445 aa, unidentified
QVHLVQSGAEVKKPGASVKVSCKASGYTFTSYGISWVRQAPGQGLEWMGWLSTYSGNTNYAQKLQGRVTMTTDTSTSTAYMELRSLRSDDTAVYYCARGNFYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 216 aa, unidentified
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 119 aa, unidentified
QVHLVQSGAEVKKPGASVKVSCKASGYTFTSYGISWVRQAPGQGLEWMGWLSTYSGNTNYAQKLQGRVTMTTDTSTSTAYMELRSLRSDDTAVYYCARGNFYYYGMDVWGQGTTVTVSS

SEQ ID NO: 12 - protein, 110 aa, unidentified
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSPPITFGQGTRLEIKR

SEQ ID NO: 13 - protein, 448 aa, unidentified
EVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLEPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVESCSVMHEALHNHYTQKSLSLSPG

SEQ ID NO: 14 - protein, 214 aa, unidentified [sequence omitted]